(19) United States
(12) Patent Application Publication
(10) Pub. No.: US 2026/0275284 A1
(43) Pub. Date: Sep. 17, 2026

(54) GENE EDITING COMBINATIONS FOR ALLOGENEIC CAR-T CELLS WITH ENHANCED IMMUNE EVASION

(71) Applicant: Kite Pharma, Inc., Santa Monica, CA (US)

(72) Inventors: Katherine A. Freitas, Redwood City, CA (US); Chloe Knights Krishnamurty, Berkeley, CA (US); Geetha Boddu, Berkeley, CA (US)

(21) Appl. No.: 19/561,932

(22) Filed: Mar. 10, 2026

Related U.S. Application Data
(60) Provisional application No. 63/770,641, filed on Mar. 12, 2025.
(60) Provisional application No. 63/920,809, filed on Nov. 19, 2025.

(51) Int. Cl. C12N 5/0783; A61K 40/11; A61K 40/31; A61K 40/42; C07K 16/28; C12N 9/22; C12N 15/11; C12N 15/90

(52) U.S. Cl. CPC C12N 5/0636; A61K 40/11; A61K 40/31; A61K 40/4204; A61K 40/4211; A61K 40/4217; A61K 40/4221; C07K 16/2803; C07K 16/2863; C07K 16/2866; C07K 16/2887; C12N 9/226; C12N 15/11; C12N 15/907; C12N 2310/20; C12N 2510/00

(57) ABSTRACT

The present disclosure provides allogeneic cells that cause reduced risks of graft-versus-host disease (GVHD) and reduced risk of CD8 and NK cell rejections, for example, when used to treat diseases, such as cancer and/or autoimmune disease, in a patient. The allogeneic cells may be genetically engineered to reduce the expression or activity of CD58, RFX5, ICAM1, FAS and/or TGFBR2. Methods of preparing and using such allogeneic cells are also provided.

### INCORPORATION BY REFERENCE

The present application claims the priority benefit of U.S. Provisional Application No. 63/770,641, filed Mar. 12, 2025, and U.S. Provisional Application No. 63/920,809, filed Nov. 19, 2025, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Feb. 27, 2026, is named K-1167-US-NP_SL.xml and is 66,660 bytes in size.

### TECHNICAL FIELD

The present disclosure relates to allogeneic cells with reduced risk of graft-versus-host disease (GVHD) and reduced risk of lymphocyte-mediated (CD4, CD8 and NK cell) rejection, for example, when used to treat diseases in a patient. The allogeneic cells may be genetically engineered to reduce the expression or activity of CD58 in combination with reduced expression of other proteins such as RFX5, FAS, ICAM1, TGFBR2, and/or T cell receptor (TCR) protein(s). The present disclosure also provides methods for preparing and using such allogeneic cells.

### BACKGROUND

Chimeric antigen receptor (CAR) modified T cells have been shown to be a promising strategy for the treatment of cancers and autoimmune diseases. CD19-directed CAR-T cells, in particular, have demonstrated potent anti-tumor efficacy in treating a range of B-cell malignancies. However, autologous CAR-T therapy presents technical, manufacturing, and commercial constraints, which may limit its clinical application to the full potential.

Allogeneic CAR-T therapy, which employs T cells from healthy human donors that subsequently undergo gene modifications to confer specificity against tumor antigens, is an alternative strategy to overcome the inherent limitations of autologous therapy and provide an “off-the-shelf” approach for clinical use. However, interactions between the T-cell receptor (TCR) on donor T cells and the mismatched human leukocyte antigen (HLA) molecules on recipient patient cells may lead to graft-versus-host disease (GVHD). Additionally, the host's endogenous CD8⁺ and CD4⁺ T cells can interact and eliminate donor T-cell grafts bearing mismatched major histocompatibility complex (MHC) class I molecules. The removal of MHC Class I results in protection from CD8⁺ T cell mediated killing, but leaves a susceptibility to NK-mediated killing due to the lack of an inhibitory signal provided by MHC I. Accordingly, there is a need to develop cells that prevent the triggering of alloreactive host T cells by disabling the expression of their human leukocyte antigen (HLA) ligands, in particular immune cells such as T and NK cells, that can achieve reduced, minimal, or no risk of GVHD and reduced, minimal, or no risk of CD4, CD8 and NK cell rejection, while retaining comparable or even improved therapeutic activities, suitable for off-the-shelf use.

### SUMMARY OF THE DISCLOSURE

Rejection by the host immune system is a critical barrier to achieving durable responses to allogeneic cell therapy. The immune response against adoptively transferred cells derived from human leukocyte antigen (HLA) mismatched donors is primarily mediated by CD4⁺ and CD8⁺ T cells. A common strategy to alleviate CD8⁺ T cell rejection is to disrupt B2M, thus preventing Class I HLA molecules from reaching the plasma membrane. However, the lack of Class I molecules triggers the cytotoxic function of the host's natural killer (NK) cells, resulting in limited persistence of the allogeneic T cells.

Herein, we describe combinations of gene knock-outs that protect from both NK cell and T cell mediated killing. This is achieved by exploiting a common dependency of lymphocytes to form a stable immune synapse mediated by CD58/CD2 and ICAM1/LFA-1 as the first step in target cell killing. Importantly, we demonstrate disruption of CD58 and ICAM1 provides dramatic protection from cellular immunity without the need to include B2M knock-out. Furthermore, we report combining FAS knockout with disruption of CD58, and/or RFX5 and/or ICAM1 diminishes killing by FAS-L presented on immune effector cells, leading to increased persistence of gene-edited allogeneic CAR-T cells. Further, we report an allogeneic T cell engineered to evade immune rejection by deletion of CD58, ICAM1, and RFX5, to have resistance to TGFβ-mediated immunosuppression by deletion of TGFBR2, and to prevent graft versus host disease by deletion of a TCR gene such as TRAC or CD3E combined with expression chimeric antigen receptors with specificity for one or more antigens.

In a first aspect, the present disclosure provides an immune cell engineered to have CD58 expression or activity that is at least 10% lower as compared to a corresponding non-engineered immune cell and to have FAS expression or activity that is at least 10% lower as compared to a corresponding non-engineered immune cell. In some embodiments, the expression of CD58 is reduced by at least 10% and the expression of FAS is reduced by at least 10%.

In some embodiments, the activity of CD58 is reduced by at least 10%. In some embodiments, the expression or activity of CD58 is reduced by at least 75%. In some embodiments, the expression of CD58 is reduced by at least 75%. In some embodiments, the activity of CD58 is reduced by at least 75%. In some embodiments, the expression or activity of CD58 is eliminated. In some embodiments, the expression of CD58 is eliminated. In some embodiments, the activity of CD58 is eliminated. In some embodiments, the activity of FAS is also reduced by at least 10%. In some embodiments, the expression or activity of FAS is reduced by at least 75%. In some embodiments, the expression of FAS is reduced by at least 75%. In some embodiments, the activity of FAS is reduced by at least 75%. In some embodiments, the expression or activity of FAS is eliminated. In some embodiments, the expression of FAS is eliminated. In some embodiments, the activity of FAS is eliminated.

In a second aspect, the present disclosure provides an immune cell engineered to have CD58 expression or activity that is at least 10% lower as compared to a corresponding non-engineered immune cell and to have ICAM1 expression or activity that is at least 10% lower as compared to a corresponding non-engineered immune cell. In some embodiments, the expression of CD58 is reduced by at least 10% and the expression of ICAM1 is reduced by at least 10%.

In some embodiments, the activity of CD58 is reduced by at least 10%. In some embodiments, the expression or activity of CD58 is reduced by at least 75%. In some embodiments, the expression of CD58 is reduced by at least 75%. In some embodiments, the activity of CD58 is reduced by at least 75%. In some embodiments, the expression or activity of CD58 is eliminated. In some embodiments, the expression of CD58 is eliminated. In some embodiments, the activity of CD58 is eliminated. In some embodiments, the activity of ICAM1 is also reduced by at least 10%. In some embodiments, the expression or activity of ICAM1 is reduced by at least 75%. In some embodiments, the expression of ICAM1 is reduced by at least 75%. In some embodiments, the activity of ICAM1 is reduced by at least 75%. In some embodiments, the expression or activity of ICAM1 is eliminated. In some embodiments, the expression of ICAM1 is eliminated. In some embodiments, the activity of ICAM1 is eliminated.

In another aspect, the present disclosure provides an immune cell engineered to have CD58 expression or activity that is at least 10% lower as compared to a corresponding non-engineered immune cell, to also have RFX5 expression or activity that is at least 10% lower as compared to a corresponding non-engineered immune cell, and that is also engineered to have TGFBR2 expression or activity that is at least 10% lower as compared to a corresponding non-engineered immune cell.

In some embodiments, the activity of CD58 is reduced by at least 10%. In some embodiments, the expression or activity of CD58 is reduced by at least 75%. In some embodiments, the expression of CD58 is reduced by at least 75%. In some embodiments, the activity of CD58 is reduced by at least 75%. In some embodiments, the expression or activity of CD58 is eliminated. In some embodiments, the expression of CD58 is eliminated. In some embodiments, the activity of CD58 is eliminated. In some embodiments, the activity of RFX5 is also reduced by at least 10%. In some embodiments, the expression or activity of RFX5 is reduced by at least 75%. In some embodiments, the expression of RFX5 is reduced by at least 75%. In some embodiments, the activity of RFX5 is reduced by at least 75%. In some embodiments, the expression or activity of RFX5 is eliminated. In some embodiments, the expression of RFX5 is eliminated. In some embodiments, the activity of RFX5 is eliminated. In some embodiments, the activity of TGFBR2 is also reduced by at least 10%. In some embodiments, the expression or activity of TGFBR2 is reduced by at least 75%. In some embodiments, the expression of TGFBR2 is reduced by at least 75%. In some embodiments, the activity of TGFBR2 is reduced by at least 75%. In some embodiments, the expression or activity of TGFBR2 is eliminated. In some embodiments, the expression of TGFBR2 is eliminated. In some embodiments, the activity of TGFBR2 is eliminated.

In some embodiments, the cell is a T cell. In some embodiments, the cell is a human cell.

In some embodiments, the cell comprises an exogenous polynucleotide encoding a chimeric antigen receptor (CAR) or a T-cell receptor (TCR). In some embodiments, the CAR recognizes CD19, CD20, CD22, BCMA, TACI, EGFRVIII, IL13RA, GPC3, GPC2, DLL3, and/or CD38. In some embodiments, the recognizes CD19 and/or CD20. In some embodiments, the CAR or TCR is introduced into the cell by transduction with a lentiviral vector. In some embodiments, the CAR or TCR is introduced into the cell prior to editing of the gene encoding CD58. In some embodiments, the TCR gene is one of TRAC, TRBC, CD3E, CD3G, CD3D, or CD3Z.

In some embodiments, the expression or activity of TRAC (T Cell Receptor Alpha Constant), TRBC, CD3E, CD3G, CD3D, and/or CD3z is also reduced in the cell. In some embodiments, the endogenous B2M (Beta-2-microglobulin) gene is not engineered, or the cell has normal expression or activity of B2M. In some embodiments, the cell has normal expression or activity of MHC Class I.

In some embodiments, the reduction in CD58 expression or activity is achieved by (a) editing of the endogenous gene encoding CD58, (b) expression of an inhibitory RNA, or (c) an inhibitor, preferably an antibody. In some embodiments, the reduction in CD58 expression or activity is achieved by editing of the endogenous gene encoding CD58. In some embodiments, the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editor.

In some embodiments, the reduction in FAS expression or activity is achieved by (a) editing of the endogenous gene encoding FAS, (b) expression of an inhibitory RNA, or (c) an inhibitor, preferably an antibody. In some embodiments, the reduction in FAS expression or activity is achieved by editing of the endogenous gene encoding FAS. In some embodiments, the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editor.

In some embodiments, the reduction in ICAM1 expression or activity is achieved by (a) editing of the endogenous gene encoding ICAM1, (b) expression of an inhibitory RNA, or (c) an inhibitor, preferably an antibody. In some embodiments, the reduction in ICAM1 expression or activity is achieved by editing of the endogenous gene encoding ICAM1. In some embodiments, the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editor.

In some embodiments, the reduction in TGFBR2 expression or activity is achieved by (a) editing of the endogenous gene encoding TGFBR2, (b) expression of an inhibitory RNA, or (c) an inhibitor, preferably an antibody. In some embodiments, the reduction in TGFBR2 expression or activity is achieved by editing of the endogenous gene encoding TGFBR2. In some embodiments, the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editor.

In some embodiments, the cell is characterized by reduced activity in inducing graft-versus-host disease (GVHD) or host rejection upon administration to a patient. In some embodiments, the cell is characterized by reduced killing by MHC-mismatched CD8⁺ T cells and/or NK cells upon administration to a patient.

In a third aspect, the present disclosure provides a method for preparing an allogeneic immune cell with reduced activity in inducing graft-versus-host disease (GVHD) or host rejection, comprising reducing, in the cell, the expression or activity of CD58 by at least 10% as compared to a corresponding non-engineered immune cell.

In some embodiments, the cell is a T cell or a natural killer (NK) cell. In some embodiments, the cell is a T cell. In some embodiments, the cell is an NK cell. In some embodiments, the cell is a human cell.

In some embodiments, the method further comprises introducing into the cell an exogenous polynucleotide encoding a chimeric antigen receptor (CAR) or a T-cell receptor (TCR). In some embodiments, the CAR recognizes CD19, CD20, CD22, BCMA, TACI, EGFRVIII, IL13RA, GPC3, GPC2, DLL3 and/or CD38. In some embodiments, the CAR recognizes CD19 and/or CD20. In some embodiments, the CAR or TCR is introduced into the cell by transduction with a lentiviral vector. In some embodiments, the CAR or TCR is introduced into the cell prior to editing of the gene encoding CD58.

In some embodiments, the reduction in CD58 expression or activity is achieved by (a) editing of the endogenous gene encoding CD58, (b) expression of an inhibitory RNA, or (c) an inhibitor, preferably an antibody. In some embodiments, the reduction in CD58 expression or activity is achieved by editing of the endogenous gene encoding CD58. In some embodiments, the editing is by CRISPR/Cas9, a zinc finger nuclease (ZFN), a transcription activator-like effector nuclease (TALEN), a MegaTAL, a meganuclease, Cpf1, homologous recombination, or a single stranded oligodeoxynucleotide (ssODN). In some embodiments, the editing is by CRISPR/Cas9. In some embodiments, the editing is by a zinc finger nuclease (ZFN).

In some embodiments, the reduction in ICAM1 expression or activity is achieved by (a) editing of the endogenous gene encoding ICAM1, (b) expression of an inhibitory RNA, or (c) an inhibitor, preferably an antibody. In some embodiments, the reduction in ICAM1 expression or activity is achieved by editing of the endogenous gene encoding ICAM1. In some embodiments, the editing is by CRISPR/Cas9, a zinc finger nuclease (ZFN), a transcription activator-like effector nuclease (TALEN), a MegaTAL, a meganuclease, Cpf1, homologous recombination, or a single stranded oligodeoxynucleotide (ssODN). In some embodiments, the editing is by CRISPR/Cas9. In some embodiments, the editing is by a zinc finger nuclease (ZFN).

In some embodiments, the reduction in FAS expression or activity is achieved by (a) editing of the endogenous gene encoding FAS, (b) expression of an inhibitory RNA, or (c) an inhibitor, preferably an antibody. In some embodiments, the reduction in FAS expression or activity is achieved by editing of the endogenous gene encoding FAS. In some embodiments, the editing is by CRISPR/Cas9, a zinc finger nuclease (ZFN), a transcription activator-like effector nuclease (TALEN), a MegaTAL, a meganuclease, Cpf1, homologous recombination, or a single stranded oligodeoxynucleotide (ssODN). In some embodiments, the editing is by CRISPR/Cas9. In some embodiments, the editing is by a zinc finger nuclease (ZFN).

In some embodiments, the reduction in TGFBR2 expression or activity is achieved by (a) editing of the endogenous gene encoding TGFBR2, (b) expression of an inhibitory RNA, or (c) an inhibitor, preferably an antibody. In some embodiments, the reduction in TGFBR2 expression or activity is achieved by editing of the endogenous gene encoding TGFBR2. In some embodiments, the editing is by CRISPR/Cas9, a zinc finger nuclease (ZFN), a transcription activator-like effector nuclease (TALEN), a MegaTAL, a meganuclease, Cpf1, homologous recombination, or a single stranded oligodeoxynucleotide (ssODN). In some embodiments, the editing is by CRISPR/Cas9. In some embodiments, the editing is by a zinc finger nuclease (ZFN).

In a fourth aspect, the present disclosure provides a method for treating cancer and/or autoimmune diseases in a patient in need thereof, comprising administering to the patient a therapeutically effective amount of a cell of the present disclosure. In some embodiments, the cell is not originally derived from the patient.

In some embodiments, the cell is administered in combination with one or more therapeutic agents.

In some embodiments, the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In some embodiments, the autoimmune disease is selected from the group comprising systemic lupus erythematosus, rheumatoid arthritis, myositis, myasthenia gravis, multiple sclerosis, Sjogren's syndrome, psoriasis, and inflammatory bowel disease.

### BRIEF DESCRIPTION OF THE DRAWINGS

FIG. 1 is a schematic depicting an allogeneic T cell engineered to evade immune rejection by deletion of CD58, FAS, ICAM1, RFX5, and a TCR gene such as TRAC or CD3E combined with expression chimeric antigen receptors with specificity for one or more antigens, according to an embodiment of the disclosure.

FIG. 2 is a series of graphs showing experimental data demonstrating highly efficient multiplex editing of 2, 3, 4, or 5 gene knockouts in human primary T cells using base editing, according to an embodiment of the disclosure.

FIG. 3 is a graph showing deletion of ICAM1 in combination with CD58 protects from CD8 T cell rejection more than CD58 deletion alone, according to an embodiment of the disclosure.

FIG. 4 is a graph showing that ICAM1 knockout alone does not protect from CD8 T cell rejection, according to an embodiment of the disclosure.

FIG. 5 is a graph showing that disruption of FAS alone does not protect from CD8 T cell rejection, according to an embodiment of the disclosure.

FIG. 6 is a graph showing superior CD8 T cell evasion in CAR-T cells with 4 gene edits compared to 2 or 3 gene edits, according to an embodiment of the disclosure.

FIG. 7 is a graph showing superior CD8 T cell evasion in CAR-T cells with 5 gene edits compared to 2 gene edits, according to an embodiment of the disclosure.

FIG. 8: is a graph showing FAS knockout in combination with CD58 and RFX5 provides protection from NK cell rejection, according to an embodiment of the disclosure.

FIG. 9 is a graph showing that loss of CD58, ICAM, FAS, and RFX5 does not impact cytotoxic function of CAR-T cells, according to an embodiment of the disclosure.

FIG. 10 is a graph showing that loss of CD58, ICAM, FAS, and RFX5 does not impair the cytokine production function of CAR-T cells, according to an embodiment of the disclosure.

FIG. 11 is a graph showing that the combination of CD58 and RFX5 knockout with ICAM1 and/or FAS knockout in CAR-T cells diminishes mismatched effector T cell survival, according to an embodiment of the disclosure.

FIG. 12 is a graph showing diminished CD8 T cell responses following exposure to CAR-T cells with 4 or 5 gene edits compared to 2 or 3 gene edits, according to an embodiment of the disclosure.

FIG. 13 is a graph showing superior NK cell evasion in CAR-T cells with 4 or 5 gene edits compared to 2 or 3 gene edits, according to an embodiment of the disclosure.

FIG. 14 is a schematic depicting an allogeneic T cell engineered to evade immune rejection by deletion of CD58, ICAM1, and RFX5, to have resistance to TGFβ-mediated immunosuppression by deletion of TGFBR2, and to prevent graft versus host disease by deletion of a TCR gene such as TRAC or CD3E combined with expression chimeric antigen receptors with specificity for one or more antigens, according to an embodiment of the disclosure.

FIG. 15 is a series of graphs showing TGFBR2 knockout efficiency assessed by loss of TGF-B1 signaling for various sgRNA sequences, according to an embodiment of the disclosures.

### DETAILED DESCRIPTION

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” may mean within one or more than one standard deviation per the practice in the art. “About” or “comprising essentially of” may mean a range of up to 10% (i.e., +10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Administering” refers to the physical introduction of an agent to a patient, such as a modified cell (e.g., a modified T cell) disclosed herein, using any of the various methods and delivery systems known to those skilled in the art. Exemplary routes of administration for the formulations disclosed herein include intravenous, for example by injection or infusion. Administering may also be performed, for example, once, a plurality of times, and/or over one or more extended periods.

The terms “activated” and “activation,” as used herein, refer to the state of a T cell that has been sufficiently stimulated to induce detect cellular proliferation. In some embodiments, activation may also be associated with induced cytokine production, and detectable effector functions. The term “activated T cells” refers to, among other things, T cells that are proliferating. Signals generated through the TCR alone may be insufficient for full activation of the T cell and one or more secondary or costimulatory signals may also be required. Thus, T cell activation comprises a primary stimulation signal through the TCR/CD3 complex and one or more secondary costimulatory signals. Costimulation may be evidenced by proliferation and/or cytokine production by T cells that have received a primary activation signal, such as stimulation through the TCR/CD3 complex.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3 with a flexible hinge region between the CH1 and CH2 domains. Each light chain comprises a light chain variable region (abbreviated herein as VL) and a light chain constant region. The light chain constant region comprises one constant domain, CL. The VH and VL regions may be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains form a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen-binding molecule,” “antigen-binding portion,” “antigen-binding fragment,” or “antibody fragment” refers to any molecule that comprises the antigen-binding parts (e.g., CDRs) of the antibody from which the molecule is derived. An antigen-binding molecule may include the antigenic complementarity determining regions (CDRs). Examples of antibody fragments include, but are not limited to, Fab, Fab′, F(ab′)2, and Fv fragments, single domain antibody (dAb), linear antibodies, scFv antibodies, and multispecific antibodies formed from antigen-binding molecules. Peptibodies (i.e., Fc fusion molecules comprising peptide binding domains) are another example of suitable antigen-binding molecules. In some embodiments, the antigen-binding molecule binds to an antigen on a tumor cell. In certain embodiments, the antigen-binding molecule is a chimeric antigen receptor (CAR) or an engineered T cell receptor (TCR). In certain embodiments, the antigen-binding molecule binds to 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), β2-integrins, BAFF, B-lymphoma cell, B cell maturation antigen (BCMA), bcr-ab1 (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DR5, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor α, Folate receptor β, Fos-related antigen 1, Fucosyl, Fucosyl GM1; GM2, ganglioside G2 (GD2), ganglioside GD3(aNeu5Ac(2-8)aNeu5Ac(2-3)bDGalp(1-4)bDGlcp(1-1) Cer), o-acetyl-GD2 ganglioside (OAcGD2), GITR (TNFRSF 18), GM1, ganglioside GM3(aNeu5Ac(2-3)bDGalp(1-4)bDGlcp(1-1) Cer), GP 100, hexasaccharide portion of globoH glycoceramide (GloboH), glycoprotein 75, Glypican-3 (GPC3), glycoprotein 100 (gp1OO), GPNMB, G protein-coupled receptor 20 (GPR20), G protein-coupled receptor class C group 5, member D (GPRC5D), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgG1, immunoglobulin lambda-like polypeptide 1 (IGLL1), IL-6, Interleukin 11 receptor alpha (IL-1 1Ra), IL-13, Interleukin-13 receptor subunit alpha-2 (IL-13Ra2 or CD213A2), insulin-like growth factor I receptor (IGF1-R), integrin α5β1, integrin αvβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-1a, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis(Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTI, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUC1), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA,NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PR1), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1). Amino acid sequences that specifically bind to said antigens are known in the art or may be prepared using methods known in the art; examples include immunoglobulins, variable regions of immunoglobulins (e.g., variable fragment (“Fv”) or bivalent variable fragment (“Fab”)), single chain antibodies, etc. In certain embodiments, the antigen-binding molecule is an antibody fragment that specifically binds to the antigen, including one or more of the complementarity determining regions (CDRs) thereof. In further embodiments, the antigen-binding molecule is a single chain variable fragment (scFv). In some embodiments, the antigen-binding molecule comprises or consists of avimers.

The terms “variable region” and “variable domain” are used interchangeably herein and typically refer to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably herein to refer to the light chain variable region of an antibody or an antigen-binding molecule thereof.

The terms “VH” and “VH domain” are used interchangeably herein to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

An “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of, e.g., a TCR or CAR contemplated herein, is designed to bind. A person of skill in the art would readily understand that any macromolecule, including virtually all proteins or peptides, may serve as an antigen. An antigen may be endogenously expressed, i.e. expressed by genomic DNA, or may be recombinantly expressed. An antigen may be specific to a certain tissue, such as a cancer cell, or it may be broadly expressed. In addition, fragments of larger molecules may act as antigens. In one embodiment, antigens are tumor antigens. In some particular embodiments, the antigen is all or a fragment of 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), β2-integrins, BAFF, B-lymphoma cell, B cell maturation antigen (BCMA), bcr-ab1 (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DR5, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor «, Folate receptor β, Fos-related antigen 1, Fucosyl, Fucosyl GM1; GM2, ganglioside G2 (GD2), ganglioside GD3 (aNeu5Ac(2-8)aNeu5Ac(2-3)bDGalp(1-4)bDGlcp(1-1) Cer), o-acetyl-GD2 ganglioside (OAcGD2), GITR (TNFRSF 18), GM1, ganglioside GM3(aNeu5Ac(2-3)bDGalp(1-4)bDGlcp(1-1) Cer), GP 100, hexasaccharide portion of globoH glycoceramide (GloboH), glycoprotein 75, Glypican-3 (GPC3), glycoprotein 100 (gp1OO), GPNMB, G protein-coupled receptor 20 (GPR20), G protein-coupled receptor class C group 5, member D (GPRC5D), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgG1, immunoglobulin lambda-like polypeptide 1 (IGLL1), IL-6, Interleukin 11 receptor alpha (IL-11Ra), IL-13, Interleukin-13 receptor subunit alpha-2 (IL-13Ra2 or CD213A2), insulin-like growth factor I receptor (IGF1-R), integrin α5β1, integrin αvβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-1a, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis(Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTI, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUC1), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA,NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PR1), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1). A “target” is any molecule bound by a binding motif, antigen binding system, CAR or antigen binding agent, e.g., an antibody.

The term “autologous” refers to any material derived from the same individual to which it is later to be re-introduced.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR-T cell. “Extracellular domain” (or “ECD”) refers to a portion of a polypeptide that, when the polypeptide is present in a cell membrane, is understood to reside outside of the cell membrane, in the extracellular space.

The term “extracellular ligand-binding domain,” as used herein, refers to an oligo- or polypeptide that is capable of binding a ligand, e.g., a cell surface molecule. For example, the extracellular ligand-binding domain may be chosen to recognize a ligand that acts as a cell surface marker on target cells associated with a particular disease state (e.g., cancer). Examples of cell surface markers that may act as ligands include those associated with viral, bacterial and parasitic infections, autoimmune disease and cancer cells.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6:412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered.

The “transmembrane” region or domain is the portion of the CAR that anchors the extracellular binding portion to the plasma membrane of the immune effector cell and facilitates binding of the binding domain to the target antigen. The transmembrane domain may be a CD28 or CD8 transmembrane domain, however other transmembrane domains that may be employed include those obtained from CD8alpha, CD4, CD28, CD45, CD9, CD16, CD22, CD33, CD64, CD80, CD86, CD134, CD137, and CD154. In some embodiments, the transmembrane domain is the transmembrane domain of CD137. In certain embodiments, the transmembrane domain is synthetic, in which case it would comprise predominantly hydrophobic residues, such as leucine and valine.

The “intracellular signaling domain” or “signaling domain” refers to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of the cell. Effector function of the T cell, for example, may be cytolytic activity or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain may be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal. The intracellular signaling domain is also known as the, “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation may be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling motifs which are known as immunoreceptor tyrosine-based activation motif or ITAMs.

Examples of ITAM containing primary cytoplasmic signaling sequences that are of particular use in the disclosure include those derived from CD3zeta, FcRgamma, FcRbeta, CD3gamma, CD3delta, CD3epsilon, CD5, CD22, CD79a, CD79b and CD66d.

As used herein, the term, “costimulatory signaling domain,” or “costimulatory domain”, refers to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1 BB (CD137), 0X40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD3zeta and 4-1 BB, other costimulatory domains are contemplated for use with the CARs described herein. The inclusion of one or more co stimulatory signaling domains may enhance the efficacy and expansion of T cells expressing CAR receptors. The intracellular signaling and costimulatory signaling domains may be linked in any order in tandem to the carboxyl terminus of the transmembrane domain.

Although scFv-based CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from CD28, CD137, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy, 2009; 17:1453-1464; Zhong et al., Molecular Therapy, 2010; 18:413-420; Carpenito et al., PNAS, 2009; 106:3360-3365).

A “costimulatory signal” refers to a signal, which in combination with a primary signal, such as TCR/CD3 ligation, leads to a T cell response, such as, but not limited to, proliferation and/or upregulation or down regulation of key molecules.

A “costimulatory ligand” includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand may include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, programmed death ligand 1 (PD-L1), or PD-L2. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD33, CD45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD11a, CD11b, CD11c, CD11d, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDI 1a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

A “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof may be replaced with an amino acid residue with a similar side chain. In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution. Exemplary amino acid categorizations are summarized in Table 1 below:

TABLE 1
Amino acid categorization

| | | | | Hydropathy |
|---|---|---|---|---|
| Amino Acid | 3-Letter | Property | Property | Index |
| Alanine | Ala | nonpolar | neutral | 1.8 |
| Arginine | Arg | polar | positive | −4.5 |
| Asparagine | Asn | polar | neutral | −3.5 |
| Aspartic acid | Asp | polar | negative | −3.5 |
| Cysteine | Cys | nonpolar | neutral | 2.5 |
| Glutamic acid | Glu | polar | negative | −3.5 |
| Glutamine | Gln | polar | neutral | −3.5 |
| Glycine | Gly | nonpolar | neutral | −0.4 |
| Histidine | His | polar | positive | −3.2 |
| Isoleucine | Ile | nonpolar | neutral | 4.5 |
| Leucine | Leu | nonpolar | neutral | 3.8 |
| Lysine | Lys | polar | positive | −3.9 |
| Methionine | Met | nonpolar | neutral | 1.9 |
| Phenylalanine | Phe | nonpolar | neutral | 2.8 |
| Proline | Pro | nonpolar | neutral | −1.6 |
| Serine | Ser | polar | neutral | −0.8 |
| Threonine | Thr | polar | neutral | −0.7 |
| Tryptophan | Trp | nonpolar | neutral | −0.9 |
| Tyrosine | Tyr | polar | neutral | −1.3 |
| Valine | Val | nonpolar | neutral | 4.2 |

A “T cell receptor” or “TCR” refers to antigen-recognition molecules present on the surface of T cells. During normal T cell development, each of the four TCR genes, α, β, γ, and δ, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

The term “host vs. graft pair” means the CAR-T cells and CD8 T cells or NK cells were isolated from different donors.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

The T cells of the immunotherapy may come from any source known in the art. For example, T cells may be differentiated in vitro from a hematopoietic stem cell population, or T cells may be obtained from a patient. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a patient using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

A “patient” includes any human who is afflicted with a cancer (e.g., a lymphoma or a leukemia) and/or an autoimmune disease. The terms “subject” and “patient” are used interchangeably herein.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition should be compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant patient or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions or eliminations.

The term “reference” describes a standard or control relative to which a comparison is performed. For example, in some embodiments, an agent, animal, cell, individual, population, sample, sequence, or value of interest is compared with a reference or control that is an agent, animal, cell, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

“Regulatory T cells” (“Treg”, “Treg cells”, or “Tregs”) refer to a lineage of CD4+T lymphocytes that participate in controlling certain immune activities, e.g., autoimmunity, allergy, and response to infection. Regulatory T cells may regulate the activities of T cell populations and may also influence certain innate immune system cell types. Tregs may be identified by the expression of the biomarkers CD4, CD25 and Foxp3, and low expression of CD127. Naturally occurring Treg cells normally constitute about 5-10% of the peripheral CD4⁺ T lymphocytes. However, within a tumor microenvironment (i.e., tumor-infiltrating Treg cells), Treg cells may make up as much as 20-30% of the total CD4⁺ T lymphocyte population.

A “therapeutically effective amount,” “effective dose,” “effective amount,” or “therapeutically effective dosage” of a therapeutic agent, e.g., engineered CAR-T cells, is any amount that, when used alone or in combination with another therapeutic agent, protects a patient against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression may be evaluated using a variety of methods known to the skilled practitioner, such as in human patients during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a patient refers to any type of intervention or process performed on, or the administration of an active agent to, the patient with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In other embodiments, “treatment” or “treating” includes a complete remission. In some embodiments, treatment may be of a patient who does not exhibit signs of the relevant disease, disorder and/or condition and/or of a patient who exhibits only early signs of the disease, disorder, and/or condition. In some embodiments, such treatment may be of a patient who exhibits one or more established signs of the relevant disease, disorder and/or condition. In some embodiments, treatment may be of a patient who has been diagnosed as suffering from the relevant disease, disorder, and/or condition. In some embodiments, treatment may be of a patient known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. Thus, each zinc finger of a multi-finger ZFP includes a recognition helix region for binding to DNA within a backbone. The term zinc finger DNA binding protein is often abbreviated as zinc finger protein or ZFP. The term “zinc finger nuclease” includes one ZFN as well as a pair of ZFNs (the members of the pair are referred to as “left and right” or “first and second” or “pair”) that dimerize to cleave the target gene.

A “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205. Zinc finger and TALE DNA-binding domains may be “engineered” to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs include design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also International Patent Publication Nos. WO 98/53058; WO 98/53059; WO 98/53060; WO 02/016536; and WO 03/016496. The term “TALEN” includes one TALEN as well as a pair of TALENs (the members of the pair are referred to as “left and right” or “first and second” or “pair”) that dimerize to cleave the target gene.

CRISPR/Cas (Clustered regularly interspaced short palindromic repeats/CRISPR-associated protein) system has been the most powerful genomic editing tool since its conception for its unparalleled editing efficiency, convenience and the potential applications in living organism. Directed by guide RNA (gRNA), a Cas nuclease can generate DNA double strand breaks (DSBs) at the targeted genomic sites in various cells (both cell lines and cells from living organisms). These DSBs are then repaired by the endogenous DNA repair system, which could be utilized to perform desired genome editing.

Base editors (BE), which integrate the CRISPR/Cas system with the APOBEC (apolipoprotein B mRNA editing enzyme, catalytic polypeptide-like) cytosine deaminase family, were recently developed that greatly enhanced the efficiency of CRISPR/Cas9-mediated gene correction. Through fusion with Cas9 nickase (nCas9) or catalytically dead Cas9 (dCas9), the cytosine (C) deamination activity of rat APOBEC1 (rA1) may be purposely directed to the target bases in genome and to catalyze C to Thymine (T) substitutions at these bases. The Base Editors (BE) may be an adenine base editor (ABE) that converts A-T to G-C or cytoside base editor (CBE) that converts cytosine-guanine (C-G) to thymine-adenine (T-A).

Prime editing (PE) is a genome editing technology by which the genome of living organisms may be modified. Prime editing directly writes new genetic information into a targeted DNA site. It uses a fusion protein, consisting of a catalytically impaired endonuclease (e.g., Cas9) fused to an engineered reverse transcriptase enzyme, and a prime editing guide RNA (pegRNA), capable of identifying the target site and providing the new genetic information to replace the target DNA nucleotides. Prime editing mediates targeted insertions, deletions, and base-to-base conversions without the need for double strand breaks (DSBs) or donor DNA templates.

As used herein, the term “agent” is used to denote a chemical compound (such as an organic or inorganic compound), a mixture of chemical compounds, a biological macromolecule (such as a nucleic acid, an antibody, a protein or portion thereof, e.g., a peptide, a lipid, or a carbohydrate) or an extract made from biological materials such as bacteria, plants, fungi, or animal (particularly mammalian) cells or tissues. Agents include, for example, agents which are known with respect to structure and/or function, and those which are not known with respect to structure or function. The activity of such agents may render it suitable as a “therapeutic agent” which is a biologically, physiologically, or pharmacologically active substance (or substances) that acts locally or systemically in a patient. Agents may comprise, for example, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes). Agents may also comprise alcohols, alkyl halides, amines, amides, esters, aldehydes, ethers and other classes of organic agents.

As used herein, the terms “nucleic acid molecule” and “polynucleotide” are used interchangeably and refer to a polymer of nucleic acid residues (“nucleotides), such as deoxyribonucleic acid (DNA) and ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotide. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, small interfering RNA (siRNA), micro-RNA, guide RNA (gRNA) cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. The term “recombinant” polynucleotide means a polynucleotide of genomic, cDNA, semi-synthetic, or synthetic origin which either does not occur in nature or is linked to another polynucleotide in a non-natural arrangement. The polynucleotide may be operatively linked to an “expression control sequence,” which refers to a nucleotide sequence that regulates the expression of a gene.

The terms “peptides”, “proteins” and “polypeptides” are used interchangeably herein and refer to a polymer of amino acid residues.

### Overview

Allogeneic donor cells from healthy donors have the potential to offer off-the-shelf cell products that may be applied on demand, at much lower costs as compared to autologous ones. With the advancement in gene editing technologies, attempts have been made to knock out or knock down certain genes in order to develop hypoimmunogenic cells suitable for off-shelf use.

Beta-2-microglobulin (B2M or B2M) is a critical component of MHC class I molecules. Deletion of B2M can eliminate MHC class I, which has been demonstrated to reduce or prevent rejection by mismatched CD8 T cells in the host. However, allogeneic anti-CD19 CAR-T cells, in which portions of both the TCR alpha constant (TRAC) locus and B2M were deleted may be susceptible to the host's NK cells, as NK cells may become stimulated and kill the allogenic T cells that lacks MHC class I expression. Accordingly, there is a need for alternative gene editing approaches that are superior to B2M knockout and can achieve minimal or no risk of graft-versus-host-disease (GVHD) and minimal or no risk of CD4, CD8 and NK cell rejections, while retaining comparable or even improved therapeutic activities. The data provided herein demonstrate that immune cells engineered to have reduced CD58 expression or activity are characterized by reduced activity in inducing graft-versus-host disease (GVHD) or host rejection and are also characterized by reduced killing by MHC-mismatched CD8⁺ T cells and/or NK cells.

CD58, FAS, ICAM1, TGFBR2, TCR Protein and/or RFX5 Knockout Cells

In a first aspect, the present disclosure provides an isolated immune cell engineered to have reduced CD58 expression or activity, engineered to have reduced FAS expression or activity, engineered to have reduced RFX5 activity, and/or engineered to have reduced ICAM1 expression or activity compared to a corresponding non-engineered immune cell. In some embodiments, CD58, RFX5, ICAM1, and/or FAS expression or activity is eliminated. In some embodiments, the CD58, RFX5, ICAM1, and/or FAS expression is reduced compared to a corresponding non-engineered immune cell. In some embodiments, the CD58, RFX5, ICAM1, and/or FAS activity is reduced compared to a corresponding non-engineered immune cell. In some embodiments, the CD58, RFX5, ICAM1, and/or FAS expression is eliminated. In some embodiments, the CD58, RFX5, ICAM1, and/or FAS activity is eliminated. In some embodiments, CD58, RFX5, ICAM1, and/or FAS expression and activity are reduced compared to a corresponding non-engineered immune cell. In some embodiments, CD58, RFX5, ICAM1, and/or FAS expression and activity are eliminated. The CD58, RFX5, ICAM1, and/or FAS expression and/or activity may be reduced or eliminated according to any of the techniques disclosed herein.

In some embodiments, the CD58, RFX5, ICAM1, and/or FAS expression or activity is reduced by at least 10% compared to a corresponding non-engineered immune cell. In some embodiments, the CD58, RFX5, ICAM1, and/or FAS expression or activity is reduced by at least 15% compared to a corresponding non-engineered immune cell. In some embodiments, the CD58, REFX5, ICAM1, and/or FAS expression or activity is reduced by at least 20% compared to a corresponding non-engineered immune cell. In some embodiments, the CD58, RFX5, ICAM1, and/or FAS expression or activity is reduced by at least 25% compared to a corresponding non-engineered immune cell. In some embodiments, the CD58, RFX5, ICAM1, and/or FAS expression or activity is reduced by at least 30% compared to a corresponding non-engineered immune cell. In some embodiments, the CD58, RFX5, ICAM1, and/or FAS expression or activity is reduced by at least 35% compared to a corresponding non-engineered immune cell. In some embodiments, the CD58, RFX5, ICAM1, and/or FAS expression or activity is reduced by at least 40% compared to a corresponding non-engineered immune cell. In some embodiments, the CD58, RFX5, ICAM1, and/or FAS expression or activity is reduced by at least 45% compared to a corresponding non-engineered immune cell. In some embodiments, the CD58, RFX5, ICAM1, and/or FAS expression or activity is reduced by at least 50% compared to a corresponding non-engineered immune cell. In some embodiments, the CD58, RFX5, ICAM1, and/or FAS expression or activity is reduced by at least 55% compared to a corresponding non-engineered immune cell. In some embodiments, the CD58, RFX5, ICAM1, and/or FAS expression or activity is reduced by at least 60% compared to a corresponding non-engineered immune cell. In some embodiments, the CD58, RFX5, ICAM1, and/or FAS expression or activity is reduced by at least 65% compared to a corresponding non-engineered immune cell. In some embodiments, the CD58, RFX5, ICAM1, and/or FAS expression or activity is reduced by at least 70% compared to a corresponding non-engineered immune cell. In some embodiments, the CD58, RFX5, ICAM1, and/or FAS expression or activity is reduced by at least 75% compared to a corresponding non-engineered immune cell. In some embodiments, the CD58, RFX5, ICAM1, and/or FAS expression or activity is reduced by at least 80% compared to a corresponding non-engineered immune cell. In some embodiments, the CD58, RFX5, ICAM1, and/or FAS expression or activity is reduced by at least 85% compared to a corresponding non-engineered immune cell. In some embodiments, the CD58, RFX5, ICAM1, and/or FAS expression or activity is reduced by at least 90% compared to a corresponding non-engineered immune cell. In some embodiments, the CD58, RFX5, ICAM1, and/or FAS expression or activity is reduced by at least 95% compared to a corresponding non-engineered immune cell. In some embodiments, the CD58, RFX5, ICAM1, and/or FAS expression or activity is reduced by at least 96% compared to a corresponding non-engineered immune cell. In some embodiments, the CD58, RFX5, ICAM1, and/or FAS expression or activity is reduced by at least 97% compared to a corresponding non-engineered immune cell. In some embodiments, the CD58, RFX5, ICAM1, and/or FAS expression or activity is reduced by at least 98% compared to a corresponding non-engineered immune cell. In some embodiments, the CD58, RFX5, ICAM1, and/or FAS expression or activity is reduced by at least 99% compared to a corresponding non-engineered immune cell.

In another aspect, the present disclosure provides an isolated immune cell engineered to have reduced CD58 expression or activity, engineered to have reduced TGFBR2 expression or activity, engineered to have reduced RFX5 activity, and/or engineered to have reduced ICAM1 expression or activity compared to a corresponding non-engineered immune cell. In some embodiments, CD58, RFX5, TGFBR2, and/or ICAM1 expression or activity is eliminated. In some embodiments, the CD58, RFX5, TGFBR2, and/or ICAM1 expression is reduced compared to a corresponding non-engineered immune cell. In some embodiments, the CD58, RFX5, TGFBR2, and/or ICAM1 activity is reduced compared to a corresponding non-engineered immune cell. In some embodiments, the CD58, RFX5, TGFBR2, and/or ICAM1 expression is eliminated. In some embodiments, the CD58, RFX5, TGFBR2, and/or ICAM1 activity is eliminated. In some embodiments, CD58, RFX5, TGFBR2, and/or ICAM1 expression and activity are reduced compared to a corresponding non-engineered immune cell. In some embodiments, CD58, RFX5, TGFBR2, and/or ICAM1 expression and activity are eliminated. The CD58, RFX5, TGFBR2, and/or ICAM1 expression and/or activity may be reduced or eliminated according to any of the techniques disclosed herein.

In some embodiments, the CD58, RFX5, TGFBR2, and/or ICAM1 expression or activity is reduced by at least 10% compared to a corresponding non-engineered immune cell. In some embodiments, the CD58, RFX5, TGFBR2, and/or ICAM1 expression or activity is reduced by at least 15% compared to a corresponding non-engineered immune cell. In some embodiments, the CD58, RFX5, TGFBR2, and/or ICAM1 expression or activity is reduced by at least 20% compared to a corresponding non-engineered immune cell. In some embodiments, the CD58, RFX5, TGFBR2, and/or ICAM1 expression or activity is reduced by at least 25% compared to a corresponding non-engineered immune cell. In some embodiments, the CD58, RFX5, TGFBR2, and/or ICAM1 expression or activity is reduced by at least 30% compared to a corresponding non-engineered immune cell. In some embodiments, the CD58, RFX5, TGFBR2, and/or ICAM1 expression or activity is reduced by at least 35% compared to a corresponding non-engineered immune cell. In some embodiments, CD58, RFX5, TGFBR2, and/or ICAM1 expression or activity is reduced by at least 40% compared to a corresponding non-engineered immune cell. In some embodiments, the CD58, RFX5, TGFBR2, and/or ICAM1 expression or activity is reduced by at least 45% compared to a corresponding non-engineered immune cell. In some embodiments, the CD58, RFX5, TGFBR2, and/or ICAM1 expression or activity is reduced by at least 50% compared to a corresponding non-engineered immune cell. In some embodiments, the CD58, RFX5, TGFBR2, and/or ICAM1 expression or activity is reduced by at least 55% compared to a corresponding non-engineered immune cell. In some embodiments, the CD58, RFX5, TGFBR2, and/or ICAM1 expression or activity is reduced by at least 60% compared to a corresponding non-engineered immune cell. In some embodiments, the CD58, RFX5, TGFBR2, and/or ICAM1 expression or activity is reduced by at least 65% compared to a corresponding non-engineered immune cell. In some embodiments, the CD58, RFX5, TGFBR2, and/or ICAM1 expression or activity is reduced by at least 70% compared to a corresponding non-engineered immune cell. In some embodiments, the CD58, RFX5, TGFBR2, and/or ICAM1 expression or activity is reduced by at least 75% compared to a corresponding non-engineered immune cell. In some embodiments, the CD58, RFX5, TGFBR2, and/or ICAM1 expression or activity is reduced by at least 80% compared to a corresponding non-engineered immune cell. In some embodiments, the CD58, RFX5, TGFBR2, and/or ICAM1 expression or activity is reduced by at least 85% compared to a corresponding non-engineered immune cell. In some embodiments, the CD58, RFX5, TGFBR2, and/or ICAM1 expression or activity is reduced by at least 90% compared to a corresponding non-engineered immune cell. In some embodiments, the CD58, RFX5, TGFBR2, and/or ICAM1 expression or activity is reduced by at least 95% compared to a corresponding non-engineered immune cell. In some embodiments, the CD58, RFX5, TGFBR2, and/or ICAM1 expression or activity is reduced by at least 96% compared to a corresponding non-engineered immune cell. In some embodiments, the CD58, RFX5, TGFBR2, and/or ICAM1 expression or activity is reduced by at least 97% compared to a corresponding non-engineered immune cell. In some embodiments, the CD58, RFX5, TGFBR2, and/or ICAM1 expression or activity is reduced by at least 98% compared to a corresponding non-engineered immune cell. In some embodiments, the CD58, RFX5, TGFBR2, and/or ICAM1 expression or activity is reduced by at least 99% compared to a corresponding non-engineered immune cell.

CD58 KO may be paired with other genetic edits such as those targeting TGFBR2, RFX5, ICAM1, and/or FAS family members (RFX5, RFANK, RFXAP), TAP1, TAP2, ICAM1, ICAM2, etc. In some embodiments, CD58 expression or activity is reduced along with the RFX5, ICAM1, TGFBR2, and/or FAS expression or activity being reduced by at least 10% compared to a corresponding non-engineered immune cell. In some embodiments, the RFX5, TGFBR2, ICAM1, and/or FAS expression or activity is reduced by at least 15% compared to a corresponding non-engineered immune cell. In some embodiments, the RFX5, TGFBR2, ICAM1, and/or FAS expression or activity is reduced by at least 20% compared to a corresponding non-engineered immune cell. In some embodiments, the RFX5, TGFBR2, ICAM1, and/or FAS expression or activity is reduced by at least 25% compared to a corresponding non-engineered immune cell. In some embodiments, the RFX5, TGFBR2, ICAM1, and/or FAS expression or activity is reduced by at least 30% compared to a corresponding non-engineered immune cell. In some embodiments, the RFX5, TGFBR2, ICAM1, and/or FAS expression or activity is reduced by at least 35% compared to a corresponding non-engineered immune cell. In some embodiments, the RFX5, TGFBR2, ICAM1, and/or FAS expression or activity is reduced by at least 40% compared to a corresponding non-engineered immune cell. In some embodiments, the RFX5, TGFBR2, ICAM1, and/or FAS expression or activity is reduced by at least 45% compared to a corresponding non-engineered immune cell. In some embodiments, the RFX5, TGFBR2, ICAM1, and/or FAS expression or activity is reduced by at least 50% compared to a corresponding non-engineered immune cell. In some embodiments, the RFX5, TGFBR2, ICAM1, and/or FAS expression or activity is reduced by at least 55% compared to a corresponding non-engineered immune cell. In some embodiments, the RFX5, TGFBR2, ICAM1, and/or FAS expression or activity is reduced by at least 60% compared to a corresponding non-engineered immune cell. In some embodiments, the RFX5, TGFBR2, ICAM1, and/or FAS expression or activity is reduced by at least 65% compared to a corresponding non-engineered immune cell. In some embodiments, the RFX5, TGFBR2, ICAM1, and/or FAS expression or activity is reduced by at least 70% compared to a corresponding non-engineered immune cell. In some embodiments, the RFX5, TGFBR2, ICAM1, and/or FAS expression or activity is reduced by at least 75% compared to a corresponding non-engineered immune cell. In some embodiments, the RFX5, TGFBR2, ICAM1, and/or FAS expression or activity is reduced by at least 80% compared to a corresponding non-engineered immune cell. In some embodiments, the RFX5, TGFBR2, ICAM1, and/or FAS expression or activity is reduced by at least 85% compared to a corresponding non-engineered immune cell. In some embodiments, the RFX5, TGFBR2, ICAM1, and/or FAS expression or activity is reduced by at least 90% compared to a corresponding non-engineered immune cell. In some embodiments, the RFX5, ICAM1, and/or FAS expression or activity is reduced by at least 95% compared to a corresponding non-engineered immune cell. In some embodiments, RFX5, TGFBR2, ICAM1, and/or FAS expression or activity is reduced by at least 96% compared to a corresponding non-engineered immune cell. In some embodiments, the RFX5, TGFBR2, ICAM1, and/or FAS expression or activity is reduced by at least 97% compared to a corresponding non-engineered immune cell. In some embodiments, the RFX5, TGFBR2, ICAM1, and/or FAS expression or activity is reduced by at least 98% compared to a corresponding non-engineered immune cell. In some embodiments, the RFX5, TGFBR2, ICAM1, and/or FAS expression or activity is reduced by at least 99% compared to a corresponding non-engineered immune cell.

In some embodiments, the CD58, RFX5, ICAM1, TGFBR2 and/or FAS expression is reduced by at least 10% compared to a corresponding non-engineered immune cell. In some embodiments, the CD58, RFX5, ICAM1, TGFBR2 and/or FAS expression is reduced by at least 15% compared to a corresponding non-engineered immune cell. In some embodiments, the CD58, RFX5, ICAM1, TGFBR2 and/or FAS expression is reduced by at least 20% compared to a corresponding non-engineered immune cell. In some embodiments, the CD58, RFX5, ICAM1, TGFBR2 and/or FAS expression is reduced by at least 25% compared to a corresponding non-engineered immune cell. In some embodiments, the CD58, RFX5, ICAM1, TGFBR2 and/or FAS expression is reduced by at least 30% compared to a corresponding non-engineered immune cell. In some embodiments, the CD58, RFX5, ICAM1, TGFBR2 and/or FAS expression is reduced by at least 35% compared to a corresponding non-engineered immune cell. In some embodiments, the CD58, RFX5, ICAM1, TGFBR2 and/or FAS expression is reduced by at least 40% compared to a corresponding non-engineered immune cell. In some embodiments, the CD58, RFX5, ICAM1, TGFBR2 and/or FAS expression is reduced by at least 45% compared to a corresponding non-engineered immune cell. In some embodiments, the CD58, RFX5, ICAM1, TGFBR2 and/or FAS expression is reduced by at least 50% compared to a corresponding non-engineered immune cell. In some embodiments, the CD58, RFX5, ICAM1, TGFBR2 and/or FAS expression is reduced by at least 55% compared to a corresponding non-engineered immune cell. In some embodiments, the CD58, RFX5, ICAM1, TGFBR2 and/or FAS expression is reduced by at least 60% compared to a corresponding non-engineered immune cell. In some embodiments, the CD58, RFX5, ICAM1, TGFBR2 and/or FAS expression is reduced by at least 65% compared to a corresponding non-engineered immune cell. In some embodiments, the CD58, RFX5, ICAM1, TGFBR2 and/or FAS expression is reduced by at least 70% compared to a corresponding non-engineered immune cell. In some embodiments, the CD58, RFX5, ICAM1, TGFBR2 and/or FAS expression is reduced by at least 75% compared to a corresponding non-engineered immune cell. In some embodiments, the CD58, RFX5, ICAM1, TGFBR2 and/or FAS expression is reduced by at least 80% compared to a corresponding non-engineered immune cell. In some embodiments, the CD58, RFX5, ICAM1, TGFBR2 and/or FAS expression is reduced by at least 85% compared to a corresponding non-engineered immune cell. In some embodiments, the CD58, RFX5, ICAM1, TGFBR2 and/or FAS expression is reduced by at least 90% compared to a corresponding non-engineered immune cell. In some embodiments, the CD58, RFX5, ICAM1, TGFBR2 and/or FAS expression is reduced by at least 95% compared to a corresponding non-engineered immune cell. In some embodiments, the CD58, RFX5, ICAM1, TGFBR2 and/or FAS expression is reduced by at least 96% compared to a corresponding non-engineered immune cell. In some embodiments, the CD58, RFX5, ICAM1, TGFBR2 and/or FAS expression is reduced by at least 97% compared to a corresponding non-engineered immune cell. In some embodiments, the CD58, RFX5, ICAM1, TGFBR2 and/or FAS expression is reduced by at least 98% compared to a corresponding non-engineered immune cell. In some embodiments, the CD58, RFX5, ICAM1, TGFBR2 and/or FAS expression is reduced by at least 99% compared to a corresponding non-engineered immune cell.

In some embodiments, the RFX5 activity is reduced by at least 10% compared to a corresponding non-engineered immune cell. In some embodiments, the RFX5 activity is reduced by at least 15% compared to a corresponding non-engineered immune cell. In some embodiments, the RFX5 activity is reduced by at least 20% compared to a corresponding non-engineered immune cell. In some embodiments, the RFX5 activity is reduced by at least 25% compared to a corresponding non-engineered immune cell. In some embodiments, the RFX5 activity is reduced by at least 30% compared to a corresponding non-engineered immune cell. In some embodiments, the RFX5 activity is reduced by at least 35% compared to a corresponding non-engineered immune cell. In some embodiments, the RFX5 activity is reduced by at least 40% compared to a corresponding non-engineered immune cell. In some embodiments, the RFX5 activity is reduced by at least 45% compared to a corresponding non-engineered immune cell. In some embodiments, the RFX5 activity is reduced by at least 50% compared to a corresponding non-engineered immune cell. In some embodiments, the RFX5 activity is reduced by at least 55% compared to a corresponding non-engineered immune cell. In some embodiments, the RFX5 activity is reduced by at least 60% compared to a corresponding non-engineered immune cell. In some embodiments, the RFX5 activity is reduced by at least 65% compared to a corresponding non-engineered immune cell. In some embodiments, the RFX5 activity is reduced by at least 70% compared to a corresponding non-engineered immune cell. In some embodiments, the RFX5 activity is reduced by at least 75% compared to a corresponding non-engineered immune cell. In some embodiments, the RFX5 activity is reduced by at least 80% compared to a corresponding non-engineered immune cell. In some embodiments, the RFX5 activity is reduced by at least 85% compared to a corresponding non-engineered immune cell. In some embodiments, the RFX5 activity is reduced by at least 90% compared to a corresponding non-engineered immune cell. In some embodiments, the RFX5 activity is reduced by at least 95% compared to a corresponding non-engineered immune cell. In some embodiments, the RFX5 activity is reduced by at least 96% compared to a corresponding non-engineered immune cell. In some embodiments, the RFX5 activity is reduced by at least 97% compared to a corresponding non-engineered immune cell. In some embodiments, the RFX5 activity is reduced by at least 98% compared to a corresponding non-engineered immune cell. In some embodiments, the RFX5 activity is reduced by at least 99% compared to a corresponding non-engineered immune cell.

In some embodiments, the FAS activity is reduced by at least 10% compared to a corresponding non-engineered immune cell. In some embodiments, the FAS activity is reduced by at least 15% compared to a corresponding non-engineered immune cell. In some embodiments, the FAS activity is reduced by at least 20% compared to a corresponding non-engineered immune cell. In some embodiments, the FAS activity is reduced by at least 25% compared to a corresponding non-engineered immune cell. In some embodiments, the FAS activity is reduced by at least 30% compared to a corresponding non-engineered immune cell. In some embodiments, the FAS activity is reduced by at least 35% compared to a corresponding non-engineered immune cell. In some embodiments, the FAS activity is reduced by at least 40% compared to a corresponding non-engineered immune cell. In some embodiments, the FAS activity is reduced by at least 45% compared to a corresponding non-engineered immune cell. In some embodiments, the FAS activity is reduced by at least 50% compared to a corresponding non-engineered immune cell. In some embodiments, the FAS activity is reduced by at least 55% compared to a corresponding non-engineered immune cell. In some embodiments, the FAS activity is reduced by at least 60% compared to a corresponding non-engineered immune cell. In some embodiments, the FAS activity is reduced by at least 65% compared to a corresponding non-engineered immune cell. In some embodiments, the FAS activity is reduced by at least 70% compared to a corresponding non-engineered immune cell. In some embodiments, the FAS activity is reduced by at least 75% compared to a corresponding non-engineered immune cell. In some embodiments, the FAS activity is reduced by at least 80% compared to a corresponding non-engineered immune cell. In some embodiments, the FAS activity is reduced by at least 85% compared to a corresponding non-engineered immune cell. In some embodiments, the FAS activity is reduced by at least 90% compared to a corresponding non-engineered immune cell. In some embodiments, the FAS activity is reduced by at least 95% compared to a corresponding non-engineered immune cell. In some embodiments, the FAS activity is reduced by at least 96% compared to a corresponding non-engineered immune cell. In some embodiments, the FAS activity is reduced by at least 97% compared to a corresponding non-engineered immune cell. In some embodiments, the FAS activity is reduced by at least 98% compared to a corresponding non-engineered immune cell. In some embodiments, the FAS activity is reduced by at least 99% compared to a corresponding non-engineered immune cell.

In some embodiments, the ICAM1 activity is reduced by at least 10% compared to a corresponding non-engineered immune cell. In some embodiments, the ICAM1 activity is reduced by at least 15% compared to a corresponding non-engineered immune cell. In some embodiments, the ICAM1 activity is reduced by at least 20% compared to a corresponding non-engineered immune cell. In some embodiments, the ICAM1 activity is reduced by at least 25% compared to a corresponding non-engineered immune cell. In some embodiments, the ICAM1 activity is reduced by at least 30% compared to a corresponding non-engineered immune cell. In some embodiments, the ICAM1 activity is reduced by at least 35% compared to a corresponding non-engineered immune cell. In some embodiments, the ICAM1 activity is reduced by at least 40% compared to a corresponding non-engineered immune cell. In some embodiments, the ICAM1 activity is reduced by at least 45% compared to a corresponding non-engineered immune cell. In some embodiments, the ICAM1 activity is reduced by at least 50% compared to a corresponding non-engineered immune cell. In some embodiments, the ICAM1 activity is reduced by at least 55% compared to a corresponding non-engineered immune cell. In some embodiments, the ICAM1 activity is reduced by at least 60% compared to a corresponding non-engineered immune cell. In some embodiments, the ICAM1 activity is reduced by at least 65% compared to a corresponding non-engineered immune cell. In some embodiments, the ICAM1 activity is reduced by at least 70% compared to a corresponding non-engineered immune cell. In some embodiments, the ICAM1 activity is reduced by at least 75% compared to a corresponding non-engineered immune cell. In some embodiments, the ICAM1 activity is reduced by at least 80% compared to a corresponding non-engineered immune cell. In some embodiments, the ICAM1 activity is reduced by at least 85% compared to a corresponding non-engineered immune cell. In some embodiments, the ICAM1 activity is reduced by at least 90% compared to a corresponding non-engineered immune cell. In some embodiments, the ICAM1 activity is reduced by at least 95% compared to a corresponding non-engineered immune cell. In some embodiments, the ICAM1 activity is reduced by at least 96% compared to a corresponding non-engineered immune cell. In some embodiments, the ICAM1 activity is reduced by at least 97% compared to a corresponding non-engineered immune cell. In some embodiments, the ICAM1 activity is reduced by at least 98% compared to a corresponding non-engineered immune cell. In some embodiments, the ICAM1 activity is reduced by at least 99% compared to a corresponding non-engineered immune cell.

In some embodiments, the TGFBR2 activity is reduced by at least 10% compared to a corresponding non-engineered immune cell. In some embodiments, the TGFBR2 activity is reduced by at least 15% compared to a corresponding non-engineered immune cell. In some embodiments, the TGFBR2 activity is reduced by at least 20% compared to a corresponding non-engineered immune cell. In some embodiments, the TGFBR2 activity is reduced by at least 25% compared to a corresponding non-engineered immune cell. In some embodiments, the TGFBR2 activity is reduced by at least 30% compared to a corresponding non-engineered immune cell. In some embodiments, the TGFBR2 activity is reduced by at least 35% compared to a corresponding non-engineered immune cell. In some embodiments, the TGFBR2 activity is reduced by at least 40% compared to a corresponding non-engineered immune cell. In some embodiments, the TGFBR2 activity is reduced by at least 45% compared to a corresponding non-engineered immune cell. In some embodiments, the TGFBR2 activity is reduced by at least 50% compared to a corresponding non-engineered immune cell. In some embodiments, the TGFBR2 activity is reduced by at least 55% compared to a corresponding non-engineered immune cell. In some embodiments, the TGFBR2 activity is reduced by at least 60% compared to a corresponding non-engineered immune cell. In some embodiments, the TGFBR2 activity is reduced by at least 65% compared to a corresponding non-engineered immune cell. In some embodiments, the TGFBR2 activity is reduced by at least 70% compared to a corresponding non-engineered immune cell. In some embodiments, the TGFBR2 activity is reduced by at least 75% compared to a corresponding non-engineered immune cell. In some embodiments, the TGFBR2 activity is reduced by at least 80% compared to a corresponding non-engineered immune cell. In some embodiments, the TGFBR2 activity is reduced by at least 85% compared to a corresponding non-engineered immune cell. In some embodiments, the TGFBR2 activity is reduced by at least 90% compared to a corresponding non-engineered immune cell. In some embodiments, the TGFBR2 activity is reduced by at least 95% compared to a corresponding non-engineered immune cell. In some embodiments, the TGFBR2 activity is reduced by at least 96% compared to a corresponding non-engineered immune cell. In some embodiments, the TGFBR2 activity is reduced by at least 97% compared to a corresponding non-engineered immune cell. In some embodiments, the TGFBR2 activity is reduced by at least 98% compared to a corresponding non-engineered immune cell. In some embodiments, the TGFBR2 activity is reduced by at least 99% compared to a corresponding non-engineered immune cell.

In some, embodiments, the cell is a T cell or a NK cell, or any other immune cell such as monocyte or macrophage, or a cell derived/differentiated from a stem cell such as an induced pluripotent stem cell (iPSC) or an embryonic stem cell. In some embodiments, the cell is a T cell. In some embodiments, the cell is a NK cell. In some embodiments the cell is a human cell. In some embodiments, the cell is derived from a healthy donor. In some embodiments, the cell is obtained from peripheral blood mononuclear cells (PBMCs), bone marrow, lymph node tissue, cord blood, thymus tissue, tissue from a site of infection, ascites, pleural effusion, spleen tissue, or a tumor. In some embodiments, the cell is differentiated in vitro from a hematopoietic stem cell population. In some embodiments, the cell is derived from one or more cell lines available in the art.

In some embodiments, the cell comprises an exogenous polynucleotide encoding a chimeric antigen receptor (CAR) or a T-cell receptor (TCR). In some embodiments, the cell comprises an exogenous polynucleotide encoding a CAR. In some embodiments, the cell comprises an exogenous polynucleotide encoding a TCR. In some embodiments, the CAR recognizes CD19, CD20, CD22, BCMA, TACI, FCRL5, BAFFR, EGFRVIII, DLL3 IL13RA, GPC3, GPC2, and/or CD38. In some embodiments, the CAR recognizes CD19. In some embodiments, the CAR recognizes CD20. In some embodiments, the CAR recognizes CD22. In some embodiments, the CAR recognizes BCMA. In some embodiments, the CAR recognizes TACI. In some embodiments, the CAR recognizes EGFRvIII. In some embodiments, the CAR recognizes IL13RA. In some embodiments, the CAR recognizes GPC3. In some embodiments, the CAR recognizes GPC2. In some embodiments, the CAR recognizes CD38. In some embodiments, the CAR recognizes DLL3. In some embodiments, the CAR recognizes FCRL5. In some embodiments, the CAR recognizes BAFFR. In some embodiments, the CAR recognizes CD19 or CD20. In some embodiments, the CAR recognizes CD19 and CD20. In some embodiments, the CAR recognizes CD19 and/or. In some embodiments, the CAR recognizes CD19. In some embodiments, the CAR recognizes CD20.

The CAR or TCR may be expressed by any method known in the art, including the methods disclosed herein. In some embodiments, the CAR or TCR is introduced into the cell by transduction with a lentiviral vector. In some embodiments, the CAR is introduced into the cell by transduction with a lentiviral vector. In some embodiments, the TCR is introduced into the cell by transduction with a lentiviral vector. In some embodiments, the CAR or TCR is introduced into the cell prior to editing of the gene encoding CD58, RFX5, ICAM1, TGFBR2 and/or FAS. In some embodiments, the CAR is introduced into the cell prior to editing of the gene encoding CD58, RFX5, ICAM1, TGFBR2 and/or FAS. In some embodiments, the TCR is introduced into the cell prior to editing of the gene encoding CD58, RFX5, ICAM1, TGFBR2 and/or FAS. In some embodiments, the CAR or TCR is introduced into the cell subsequent to editing of the gene encoding CD58, RFX5, ICAM1, TGFBR2 and/or FAS. In some embodiments, the CAR is introduced into the cell subsequent to editing of the gene encoding CD58, RFX5, ICAM1, TGFBR2 and/or FAS. In some embodiments, the TCR is introduced into the cell subsequent to editing of the gene encoding CD58. In some embodiments, the CAR or TCR is introduced into the cell simultaneously with editing of the gene encoding CD58, RFX5, ICAM1, TGFBR2 and/or FAS. In some embodiments, the CAR is introduced into the cell simultaneously with editing of the gene encoding CD58, RFX5, ICAM1, TGFBR2 and/or FAS. In some embodiments, the TCR is introduced into the cell simultaneously with editing of the gene encoding CD58, RFX5, ICAM1, TGFBR2 and/or FAS.

In some embodiments, the cell is further engineered to reduce the expression and/or activity of TRAC (T Cell Receptor Alpha Constant), TRBC, CD3E, CD3D, CD3G, and/or CD3Z. In some embodiments, the cell is further engineered to reduce the expression of TRAC, TRBC, CD3E, CD3D, CD3G, and/or CD3Z. In some embodiments, the cell is further engineered to reduce the expression and activity of TRAC, TRBC, CD3E, CD3D, CD3G, and/or CD3Z. In some embodiments, the cell is further engineered to eliminate the expression and/or activity of TRAC, TRBC, CD3E, CD3D, CD3G, and/or CD3Z. In some embodiments, the cell is further engineered to eliminate the expression of TRAC, TRBC, CD3E, CD3D, CD3G, and/or CD3Z. In some embodiments, the cell is further engineered to eliminate the expression activity of TRAC, TRBC, CD3E, CD3D, CD3G, and/or CD3Z. In some embodiments, the cell is further engineered to eliminate the expression and activity of TRAC, TRBC, CD3E, CD3D, CD3G, and/or CD3Z.

In some embodiments, the endogenous B2M (Beta-2-microglobulin) gene is not engineered. That is, no gene editing is conducted to the B2M locus and no inhibitory agent is introduced to the cell. In some embodiments, the cell retains normal activity of B2M. In some embodiments, the cell retains normal activity of MHC Class I.

In some embodiments, the cell is characterized by reduced activity in inducing graft-versus-host disease (GVHD) or host rejection upon administration to a host. In some embodiments, the cell is characterized by reduced activity in inducing GVHD upon administration to a host. In some embodiments, the cell is characterized by reduced activity in inducing host rejection upon administration to a host. In some embodiments, the cell is characterized by reduced activity in inducing GVHD and host rejection upon administration to a host. In some embodiments, the cell is characterized by reduced killing by MHC-mismatched CD8⁺ T cells and/or NK cells upon administration to a host. In some embodiments, the cell is characterized by reduced killing by MHC-mismatched CD8⁺ T cells or NK cells upon administration to a host. In some embodiments, the cell is characterized by reduced killing by MHC-mismatched CD8⁺ T cells and NK cells upon administration to a host. In some embodiments, the cell is characterized by reduced killing by MHC-mismatched CD8⁺ T cells upon administration to a host. In some embodiments, the cell is characterized by reduced killing by MHC-mismatched NK cells upon administration to a host.

### Techniques for Reducing the Expression/Activity of CD58, RFX5, ICAM1, TGFBR2 and/or FAS in a Cell

In a second aspect, the present disclosure provides a method for preparing an allogeneic immune cell with reduced activity in inducing graft-versus-host disease (GVHD) or host rejection, comprising reducing, in the cell, the expression or activity of CD58, RFX5, ICAM1, TGFBR2 and/or FAS compared to a corresponding non-engineered immune cell. In some embodiments, the method prepares an allogeneic immune cell with reduced activity in inducing GVHD compared to a corresponding non-engineered immune cell. In some embodiments, the method prepares an allogeneic immune cell with reduced activity in inducing host rejection compared to a corresponding non-engineered immune cell. In some embodiments, the CD58, RFX5, ICAM1, TGFBR2 and/or FAS expression is reduced. In some embodiments, the CD58 activity is reduced. In some embodiments, the CD58, RFX5, ICAM1, TGFBR2 and/or FAS expression and activity are reduced. In some embodiments, the CD58, RFX5, ICAM1, TGFBR2 and/or FAS expression is eliminated. In some embodiments, the CD58, RFX5, ICAM1, TGFBR2 and/or FAS activity is eliminated. In some embodiments, the CD58, RFX5, ICAM1, TGFBR2 and/or FAS expression or activity is eliminated. In some embodiments, the CD58, RFX5, ICAM1, TGFBR2 and/or FAS expression and activity are eliminated.

Any methods of reducing or eliminating the expression or activity of a gene known in the art may be used. In some embodiments, such reduction or elimination includes any detectable decrease in the production CD58, RFX5, ICAM1, TGFBR2 and/or FAS as compared to a control (such an amount of CD58, RFX5, ICAM1, TGFBR2 and/or FAS detected in a corresponding cell in which the CD58, RFX5, ICAM1, TGFBR2 and/or FAS has not been inhibited). In some embodiments, detectable CD58, RFX5, ICAM1, TGFBR2 and/or FAS in a cell decreases by at least 10%. In some embodiments, detectable CD58, RFX5, ICAM1, TGFBR2 and/or FAS in a cell decreases by at least 15%. In some embodiments, detectable CD58, RFX5, ICAM1, TGFBR2 and/or FAS in a cell decreases by at least 20%. In some embodiments, detectable CD58, RFX5, ICAM1, TGFBR2 and/or FAS in a cell decreases by at least 25%. In some embodiments, detectable CD58, RFX5, ICAM1, TGFBR2 and/or FAS in a cell decreases by at least 30%. In some embodiments, detectable CD58, RFX5, ICAM1, TGFBR2 and/or FAS in a cell decreases by at least 35%. In some embodiments, detectable CD58, RFX5, ICAM1, TGFBR2 and/or FAS in a cell decreases by at least 40%. In some embodiments, detectable CD58, RFX5, ICAM1, TGFBR2 and/or FAS in a cell decreases by at least 45%. In some embodiments, detectable CD58, RFX5, ICAM1, TGFBR2 and/or FAS in a cell decreases by at least 50%. In some embodiments, detectable CD58, RFX5, ICAM1, TGFBR2 and/or FAS in a cell decreases by at least 55%. In some embodiments, detectable CD58, RFX5, ICAM1, TGFBR2 and/or FAS in a cell decreases by at least 60%. In some embodiments, detectable CD58, RFX5, ICAM1, TGFBR2 and/or FAS in a cell decreases by at least 65%. In some embodiments, detectable CD58, RFX5, ICAM1, TGFBR2 and/or FAS in a cell decreases by at least 70%. In some embodiments, detectable CD58, RFX5, ICAM1, TGFBR2 and/or FAS in a cell decreases by at least 75%, In some embodiments, detectable CD58, RFX5, ICAM1, TGFBR2 and/or FAS in a cell decreases by at least 80%. In some embodiments, detectable CD58, RFX5, ICAM1, TGFBR2 and/or FAS in a cell decreases by at least 85%. In some embodiments, detectable CD58, RFX5, ICAM1, TGFBR2 and/or FAS in a cell decreases by at least 90%. In some embodiments, detectable CD58, RFX5, ICAM1, TGFBR2 and/or FAS in a cell decreases by at least 95%. In some embodiments, detectable CD58, RFX5, ICAM1, TGFBR2 and/or FAS in a cell decreases by at least 96%. In some embodiments, detectable CD58, RFX5, ICAM1, TGFBR2 and/or FAS in a cell decreases by at least 97%. In some embodiments, detectable CD58, RFX5, ICAM1, TGFBR2 and/or FAS in a cell decreases by at least 98%. In some embodiments, detectable CD58, RFX5, ICAM1, TGFBR2 and/or FAS in a cell decreases by at least 99%.

In some embodiments, CD58, RFX5, ICAM1, TGFBR2 and/or FAS expression in a cell decreases by at least 10%. In some embodiments, CD58, RFX5, ICAM1, TGFBR2 and/or FAS expression in a cell decreases by at least 15%. In some embodiments, CD58, RFX5, ICAM1, TGFBR2 and/or FAS expression in a cell decreases by at least 20%. In some embodiments, CD58, RFX5, ICAM1, TGFBR2 and/or FAS expression in a cell decreases by at least 25%. In some embodiments, CD58, RFX5, ICAM1, TGFBR2 and/or FAS expression in a cell decreases by at least 30%. In some embodiments, CD58, RFX5, ICAM1, TGFBR2 and/or FAS expression in a cell decreases by at least 35%. In some embodiments, CD58, RFX5, ICAM1, TGFBR2 and/or FAS expression in a cell decreases by at least 40%. In some embodiments, CD58, RFX5, ICAM1, TGFBR2 and/or FAS expression in a cell decreases by at least 45%. In some embodiments, CD58, RFX5, ICAM1, TGFBR2 and/or FAS expression in a cell decreases by at least 50%. In some embodiments, CD58, RFX5, ICAM1, TGFBR2 and/or FAS expression in a cell decreases by at least 55%. In some embodiments, CD58, RFX5, ICAM1, TGFBR2 and/or FAS expression in a cell decreases by at least 60%. In some embodiments, CD58, RFX5, ICAM1, TGFBR2 and/or FAS expression in a cell decreases by at least 65%. In some embodiments, CD58, RFX5, ICAM1, TGFBR2 and/or FAS expression in a cell decreases by at least 70%. In some embodiments, CD58, RFX5, ICAM1, TGFBR2 and/or FAS expression in a cell decreases by at least 75%. In some embodiments, CD58, RFX5, ICAM1, TGFBR2 and/or FAS expression in a cell decreases by at least 80%. In some embodiments, CD58, RFX5, ICAM1, TGFBR2 and/or FAS expression in a cell decreases by at least 85%. In some embodiments, CD58, RFX5, ICAM1, TGFBR2 and/or FAS expression in a cell decreases by at least 90%. In some embodiments, CD58, RFX5, ICAM1, TGFBR2 and/or FAS expression in a cell decreases by at least 95%. In some embodiments, CD58, RFX5, ICAM1, TGFBR2 and/or FAS expression in a cell decreases by at least 96%. In some embodiments, CD58, RFX5, ICAM1, TGFBR2 and/or FAS expression in a cell decreases by at least 97%. In some embodiments, CD58, RFX5, ICAM1, TGFBR2 and/or FAS expression in a cell decreases by at least 98%. In some embodiments, CD58, RFX5, ICAM1, TGFBR2 and/or FAS expression in a cell decreases by at least 99%.

In some embodiments, CD58, RFX5, ICAM1, TGFBR2 and/or FAS activity in a cell decreases by at least 10%. In some embodiments, CD58, RFX5, ICAM1, TGFBR2 and/or FAS activity in a cell decreases by at least 15%. In some embodiments, CD58, RFX5, ICAM1, TGFBR2 and/or FAS activity in a cell decreases by at least 20%. In some embodiments, CD58, RFX5, ICAM1, TGFBR2 and/or FAS activity in a cell decreases by at least 25%. In some embodiments, CD58, RFX5, ICAM1, TGFBR2 and/or FAS activity in a cell decreases by at least 30%. In some embodiments, CD58, RFX5, ICAM1, TGFBR2 and/or FAS activity in a cell decreases by at least 35%. In some embodiments, CD58, RFX5, ICAM1, TGFBR2 and/or FAS activity in a cell decreases by at least 40%. In some embodiments, CD58, RFX5, ICAM1, TGFBR2 and/or FAS activity in a cell decreases by at least 45%. In some embodiments, CD58, RFX5, ICAM1, TGFBR2 and/or FAS activity in a cell decreases by at least 50%. In some embodiments, CD58, RFX5, ICAM1, TGFBR2 and/or FAS activity in a cell decreases by at least 55%. In some embodiments, CD58, RFX5, ICAM1, TGFBR2 and/or FAS activity in a cell decreases by at least 60%. In some embodiments, CD58, RFX5, ICAM1, TGFBR2 and/or FAS activity in a cell decreases by at least 65%. In some embodiments, CD58, RFX5, ICAM1, TGFBR2 and/or FAS activity in a cell decreases by at least 70%. In some embodiments, CD58, RFX5, ICAM1, TGFBR2 and/or FAS activity in a cell decreases by at least 75%. In some embodiments, CD58, RFX5, ICAM1, TGFBR2 and/or FAS activity in a cell decreases by at least 80%. In some embodiments, CD58, RFX5, ICAM1, TGFBR2 and/or FAS activity in a cell decreases by at least 85%. In some embodiments, CD58, RFX5, ICAM1, TGFBR2 and/or FAS activity in a cell decreases by at least 90%. In some embodiments, CD58, RFX5, ICAM1, TGFBR2 and/or FAS activity in a cell decreases by at least 95%. In some embodiments, CD58, RFX5, ICAM1, TGFBR2 and/or FAS activity in a cell decreases by at least 96%. In some embodiments, CD58, RFX5, ICAM1, TGFBR2 and/or FAS activity in a cell decreases by at least 97%. In some embodiments, CD58, RFX5, ICAM1, TGFBR2 and/or FAS activity in a cell decreases by at least 98%. In some embodiments, CD58, RFX5, ICAM1, TGFBR2 and/or FAS activity in a cell decreases by at least 99%.

In some embodiments, the reduction in CD58, RFX5, ICAM1, TGFBR2 and/or FAS expression or activity is achieved by (a) editing of the endogenous gene encoding CD58, RFX5, ICAM1, TGFBR2 and/or FAS, (b) expression of an inhibitory RNA, or (c) an inhibitor, preferably an antibody. In some embodiments, the expression or activity of a gene may be reduced with a suitable inhibiting agent, such as a small molecule inhibitor, an inhibitory RNA (e.g., siRNA, shRNA, or miRNA), or an antibody that targets CD58, RFX5, ICAM1, TGFBR2 and/or FAS. In some embodiments, the inhibiting agent is an inhibitory RNA that targets the CD58, RFX5, ICAM1, TGFBR2 and/or FAS mRNA. In some embodiments, the inhibitory RNA is selected from the group consisting of a siRNA, a shRNA, and a miRNA. In some embodiments, the inhibitory RNA is a siRNA. In some embodiments, the inhibitory RNA is a shRNA. In some embodiments, the inhibitory RNA is a miRNA. In some embodiments, the inhibiting agent is an antibody that targets the CD58, REX5, ICAM1, TGFBR2 and/or FAS. In some embodiments, the reduction in CD58, RFX5, ICAM1, TGFBR2 and/or FAS expression or activity is achieved by genetic editing of the CD58, RFX5, ICAM1, TGFBR2 and/or FAS gene, at one or both of the alleles of the CD58, RFX5, ICAM1, TGFBR2 and/or FAS gene.

In certain embodiments, the expression of CD58, RFX5, ICAM1, TGFBR2 and/or FAS is reduced using a DNA-binding domain, for example coupled to a nuclease domain, that specifically binds to a target site in the CD58, RFX5, ICAM1, TGFBR2 and/or FAS gene or an associated expression regulation sequence and mediates mutation at the target site thereby decreasing expression of functional CD58, RFX5, ICAM1, TGFBR2 and/or FAS. Any DNA-binding domain may be used in the compositions and methods disclosed herein, including but not limited to a zinc finger DNA-binding domain, a transcription activator-like effector (TALE) DNA binding domain, the DNA-binding portion (sgRNA) of a CRISPR/Cas nuclease, or a DNA-binding domain from a meganuclease. In certain embodiments, the expression of CD58, RFX5, ICAM1, TGFBR2 and/or FAS is reduced using a DNA-binding domain, for example coupled to a nuclease domain, that specifically binds to a target site in the CD58, RFX5, ICAM1, TGFBR2 and/or FAS gene or an associated expression regulation sequence and mediates mutation at the target site thereby decreasing expression of functional CD58, RFX5, ICAM1, TGFBR2 and/or FAS. Any DNA-binding domain may be used in the compositions and methods disclosed herein, including but not limited to a zinc finger DNA-binding domain, a transcription activator-like effector (TALE) DNA binding domain, the DNA-binding portion (sgRNA) of a CRISPR/Cas nuclease, or a DNA-binding domain from a meganuclease.

In some embodiments, reduction or elimination includes any detectable decrease in the production of a gene (e.g., CD58, RFX5, ICAM1, TGFBR2 and/or FAS). In certain examples, detectable CD58, RFX5, ICAM1, TGFBR2 and/or FAS in a cell decreases by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of CD58, RFX5, ICAM1, TGFBR2 and/or FAS detected in a corresponding cell in which the CD58, RFX5, ICAM1, TGFBR2 and/or FAS has not been inhibited).

In certain embodiments, reduction or elimination of gene expression occurs by direct inhibition of the gene (e.g., knocking down or knocking out the CD58, RFX5, ICAM1, TGFBR2 and/or FAS gene may reduce or eliminate expression or activity of CD58, RFX5, ICAM1, TGFBR2 and/or FAS). In other embodiments, reduction or elimination of gene expression occurs by indirect inhibition of the gene (e.g., knocking down or knocking out the CD58, RFX5, ICAM1, TGFBR2 and/or FAS gene may reduce expression or activity of MHC class I molecules).

Percent decrease and percent increases can be calculated by methods known in the art. As a non-liming example, a percent reduction or decrease in expression or activity of a molecule in an edited cell (e.g., a cell comprising an CD58, RFX5, ICAM1, TGFBR2 and/or FAS KO) relative to a reference or corresponding cell (e.g., a cell that does not comprise an CD58, RFX5, ICAM1, TGFBR2 and/or FAS KO) may be calculated by subtracting the reference/corresponding cell value minus the edited cell value, dividing that amount the reference value, and then multiplying by 100 to get a percent decrease. If the percent is negative, that may mean that there was an increase and not a decrease.

In certain embodiments, the expression of one or more of CD58, RFX5, ICAM1, TGFBR2 and/or FAS is reduced using a DNA-binding domain, for example coupled to a nuclease domain, that specifically binds to a target site in the CD58, RFX5, ICAM1, TGFBR2 and/or FAS gene and mediates mutation at the target site thereby decreasing expression of functional CD58, RFX5, ICAM1, TGFBR2 and/or FAS. Any DNA-binding domain can be used in the compositions and methods disclosed herein, including but not limited to a zinc finger DNA-binding domain, a TALE DNA binding domain, the DNA-binding portion (sgRNA) of a CRISPR/Cas nuclease, or a DNA-binding domain from a meganuclease.

In certain embodiments, the DNA binding domain comprises a zinc finger protein. Preferably, the zinc finger protein is non-naturally occurring in that it is engineered to bind to a target site of choice. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence.

Usually, the ZFPs include at least three fingers. Certain of the ZFPs include four, five or six fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs may also be fusion proteins that include one or more regulatory domains, which domains may be transcriptional activation or repression domains.

In some embodiments, the DNA-binding domain may be derived from a nuclease. For example, the recognition sequences of homing endonucleases and meganucleases such as I-Scel, I-Ceul, PI-PspI, PI-Sce, I-SceIV, I-CsmI, I-PanI, I-SceII, I-PpoI, I-SceIII, I-CreI, I-TevI, I-TevII and I-TevIII are known. In addition, the DNA-binding specificity of homing endonucleases and meganucleases may be engineered to bind non-natural target sites.

In some embodiments, the transcription activator-like effector nuclease (TALEN) comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity.

In certain embodiments, the DNA-binding domain is part of a CRISPR/Cas nuclease system, including a single guide RNA (sgRNA) that binds to DNA. The CRISPR (clustered regularly interspaced short palindromic repeats) locus, which encodes RNA components of the system, and the cas (CRISPR-associated) locus, which encodes proteins make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

Singe guide RNAs (sgRNAs or gRNAs) that may be suitable for use in the cells and methods of the present disclosure may be identified using CRISPR design tools. Exemplary gRNA spacer sequences are shown in Table 2. sgRNAs including the spacer sequences in Table 2 will cause gene knockout when co-expressed with a Cas9-based adenine base editor such as ABE8.

TABLE 2
Exemplary guide RNAs

| Target | | |
|---|---|---|
| Gene | Spacer Sequence | sgRNA sequence |
| B2M | ACTCACGCTGGATAG | ACTCACGCTGGATAG |
| | CCTCC | CCTCCgtttaagagc |
| | (SEQ ID NO: 1) | taagctggaaacagc |
| | | atagcaagtttaaat |
| | | aaggctagtccgtta |
| | | tcaacttgaaaaagt |
| | | ggcaccgagtcggtg |
| | | cttttttt |
| | | (SEQ ID NO: 2) |
| CD58 | CTCACCGCTGCTTGG | CTCACCGCTGCTTGG |
| | GATAC | GATACgtttaagagc |
| | (SEQ ID NO: 3) | taagctggaaacagc |
| | | atagcaagtttaaat |
| | | aaggctagtccgtta |
| | | tcaacttgaaaaagt |
| | | ggcaccgagtcggtg |
| | | cttttttt |
| | | (SEQ ID NO: 4) |
| RFX5 | GTACTTACGAAATGG | GTACTTACGAAATGG |
| | TACCT | TACCTgtttaagagc |
| | (SEQ ID NO: 5) | taagctggaaacagc |
| | | atagcaagtttaaat |
| | | aaggctagtccgtta |
| | | tcaacttgaaaaagt |
| | | ggcaccgagtcggtg |
| | | cttttttt |
| | | (SEQ ID NO: 6) |
| CD3E | CTGGATTACCTCTTG | CTGGATTACCTCTTG |
| | CCCTC | CCCTCgtttaagagc |
| | (SEQ ID NO: 7) | taagctggaaacagc |
| | | atagcaagtttaaat |
| | | aaggctagtccgtta |
| | | tcaacttgaaaaagt |
| | | ggcaccgagtcggtg |
| | | cttttttt |
| | | (SEQ ID NO: 8) |
| FAS | AACTTACCCCAAACA | AACTTACCCCAAACA |
| | ATTAG | ATTAGgtttaagagc |
| | (SEQ ID NO: 9) | taagctggaaacagc |
| | | atagcaagtttaaat |
| | | aaggctagtccgtta |
| | | tcaacttgaaaaagt |
| | | ggcaccgagtcggtg |
| | | cttttttt |
| | | (SEQ ID NO: 10) |
| ICAM1 | TCCTGCAGGGACTCC | TCCTGCAGGGACTCC |
| | AGAAC | AGAACgtttaagagc |
| | (SEQ ID NO: 11) | taagctggaaacagc |
| | | atagcaagtttaaat |
| | | aaggctagtccgtta |
| | | tcaacttgaaaaagt |
| | | ggcaccgagtcggtg |
| | | cttttttt |
| | | (SEQ ID NO: 12) |
| AAVS1 | GTTAATGTGGCTCTG | GTTAATGTGGCTCTG |
| | GTTCT | GTTCTgtttaagagc |
| | (SEQ ID NO: 13) | taagctggaaacagc |
| | | atagcaagtttaaat |
| | | aaggctagtccgtta |
| | | tcaacttgaaaaagt |
| | | ggcaccgagtcggtg |
| | | cttttttt |
| | | (SEQ ID NO: 14) |

Amino acid sequences of the target proteins that are described herein are provided in Table 3.

TABLE 3
Protein Sequences

| | Target | |
|---|---|---|
| | Gene | Sequence |
| SEQ ID | FAS | MLGIWTLLPLVLTSVARLSSKSVNAQVTD |
| NO: 15 | | INSKGLELRKTVTTVETQNLEGLHHDGQF |
| | | CHKPCPPGERKARDCTVNGDEPDCVPCQE |
| | | GKEYTDKAHFSSKCRRCRLCDEGHGLEVE |
| | | INCTRTQNTKCRCKPNFFCNSTVCEHCDP |
| | | CTKCEHGIIKECTLTSNTKCKEEGSRSNL |
| | | GWLCLLLLPIPLIVWVKRKEVQKTCRKHR |
| | | KENQGSHESPTLNPETVAINLSDVDLSKY |
| | | ITTIAGVMTLSQVKGFVRKNGVNEAKIDE |
| | | IKNDNVQDTAEQKVQLLRNWHQLHGKKEA |
| | | YDTLIKDLKKANLCTLAEKIQTIILKDIT |
| | | SDSENSNFRNEIQSLV |
| SEQ ID | ICAM1 | MAPSSPRPALPALLVLLGALFPGPGNAQT |
| NO: 16 | | SVSPSKVILPRGGSVLVTCSTSCDQPKLL |
| | | GIETPLPKKELLLPGNNRKVYELSNVQED |
| | | SQPMCYSNCPDGQSTAKTFLTVYWTPERV |
| | | ELAPLPSWQPVGKNLTLRCQVEGGAPRAN |
| | | LTVVLLRGEKELKREPAVGEPAEVTTTVL |
| | | VRRDHHGANFSCRTELDLRPQGLELFENT |
| | | SAPYQLQTFVLPATPPQLVSPRVLEVDTQ |
| | | GTVVCSLDGLFPVSEAQVHLALGDQRLNP |
| | | TVTYGNDSFSAKASVSVTAEDEGTQRLTC |
| | | AVILGNQSQETLQTVTIYSFPAPNVILTK |
| | | PEVSEGTEVTVKCEAHPRAKVTLNGVPAQ |
| | | PLGPRAQLLLKATPEDNGRSFSCSATLEV |
| | | AGQLIHKNQTRELRVLYGPRLDERDCPGN |
| | | WTWPENSQQTPMCQAWGNPLPELKCLKDG |
| | | TFPLPIGESVTVTRDLEGTYLCRARSTQG |
| | | EVTRKVTVNVLSPRYEIVIITVVAAAVIM |
| | | GTAGLSTYLYNRQRKIKKYRLQQAQKGTP |
| | | MKPNTQATPP |
| SEQ ID | CD58 | MVAGSDAGRALGVLSVVCLLHCFGFISCF |
| NO: 17 | | SQQIYGVVYGNVTFHVPSNVPLKEVLWKK |
| | | QKDKVAELENSEFRAFSSFKNRVYLDTVS |
| | | GSLTIYNLTSSDEDEYEMESPNITDTMKF |
| | | FLYVLESLPSPTLTCALTNGSIEVQCMIP |
| | | EHYNSHRGLIMYSWDCPMEQCKRNSTSIY |
| | | FKMENDLPQKIQCTLSNPLFNTTSSIILT |
| | | TCIPSSGHSRHRYALIPIPLAVITTCIVL |
| | | YMNGILKCDRKPDRTNSN |
| SEQ ID | RFX5 | MAEDEPDAKSPKTGGRAPPGGAEAGEPTTL |
| NO: 18 | | LQRLRGTISKAVQNKVEGILQDVQKFSDND |
| | | KLYLYLQLPSGPTTGDKSSEPSTLSNEEYM |
| | | YAYRWIRNHLEEHTDTCLPKQSVYDAYRKY |
| | | CESLACCRPLSTANFGKIIREIFPDIKARR |
| | | LGGRGQSKYCYSGIRRKTLVSMPPLPGLDL |
| | | KGSESPEMGPEVTPAPRDELVEAACALTCD |
| | | WAERILKRSESSIVEVARFLLQQHLISARS |
| | | AHAHVLKAMGLAEEDEHAPRERSSKPKNGL |
| | | ENPEGGAHKKPERLAQPPKDLEARTGAGPL |
| | | ARGERKKSVVESSAPGANNLQVNALVARLP |
| | | LLLPRAPRSLIPPIPVSPPILAPRLSSGAL |
| | | KVATLPLSSRAGAPPAAVPIINMILPTVPA |
| | | LPGPGPGPGRAPPGGLTQPRGTENREVGIG |
| | | GDQGPHDKGVKRTAEVPVSEASGQAPPAKA |
| | | AKQDIEDTASDAKRKRGRPRKKSGGSGERN |
| | | STPLKSAAAMESAQSSRLPWETWGSGGEGN |
| | | SAGGAERPGPMGEAEKGAVLAQGQGDGTVS |
| | | KGGRGPGSQHTKEAEDKIPLVPSKVSVIKG |
| | | SRSQKEAFPLAKGEVDTAPQGNKDLKEHVL |
| | | QSSLSQEHKDPKATPP |
| SEQ ID | CD3E | MQSGTHWRVLGLCLLSVGVWGQDGNEEMGG |
| NO: 19 | | ITQTPYKVSISGTTVILTCPQYPGSEILWQ |
| | | HNDKNIGGDEDDKNIGSDEDHLSLKEFSEL |
| | | EQSGYYVCYPRGSKPEDANFYLYLRARVCE |
| | | NCMEMDVMSVATIVIVDICITGGLLLLVYY |
| | | WSKNRKAKAKPVTRGAGAGGRQRGQNKERP |
| | | PPVPNPDYEPIRKGQRDLYSGLNQRRI |
| SEQ ID | CD3G | MEQGKGLAVLILAIILLQGTLAQSIKGNHL |
| NO: 20 | | VKVYDYQEDGSVLLTCDAEAKNITWFKDGK |
| | | MIGFLTEDKKKWNLGSNAKDPRGMYQCKGS |
| | | QNKSKPLQVYYRMCQNCIELNAATISGFLF |
| | | AEIVSIFVLAVGVYFIAGQDGVRQSRASDK |
| | | QTLLPNDQLYQPLKDREDDQYSHLQGNQLR |
| | | RN |
| SEQ ID | CD3D | MEHSTFLSGLVLATLLSQVSPFKIPIEELE |
| NO: 21 | | DRVFVNCNTSITWVEGTVGTLLSDITRLDL |
| | | GKRILDPRGIYRCNGTDIYKDKESTVQVHY |
| | | RMCQSCVELDPATVAGIIVTDVIATLLLAL |
| | | GVFCFAGHETGRLSGAADTQALLRNDQVYQ |
| | | PLRDRDDAQYSHLGGNWARNK |
| SEQ ID | TRAC | IQNPDPAVYQLRDSKSSDKSVCLFTDFDSQ |
| NO: 22 | | TNVSQSKDSDVYITDKTVLDMRSMDFKSNS |
| | | AVAWSNKSDFACANAFNNSIIPEDTFFPSP |
| | | ESSCDVKLVEKSFETDTNLNFQNLSVIGFR |
| | | ILLLKVAGFNLLMTLRLWSS |
| SEQ ID | TRBC | DLNKVFPPEVAVFEPSEAEISHTQKATLVC |
| NO: 23 | | LATGFFPDHVELSWWVNGKEVHSGVSTDPQ |
| | | PLKEQPALNDSRYCLSSRLRVSATFWQNPR |
| | | NHFRCQVQFYGLSENDEWTQDRAKPVTQIV |
| | | SAEAWGRADCGFTSVSYQQGVLSATILYEI |
| | | LLGKATLYAVLVSALVLMAMVKRKDF |
| SEQ ID | TGFBR2 | MGRGLLRGLWPLHIVLWTRIASTIPPHVQK |
| NO: 66 | | SVNNDMIVTDNNGAVKFPQLCKFCDVRFST |
| | | CDNQKSCMSNCSITSICEKPQEVCVAVWRK |
| | | NDENITLETVCHDPKLPYHDFILEDAASPK |
| | | CIMKEKKKPGETFFMCSCSSDECNDNIIFS |
| | | EEYNTSNPDLLLVIFQVTGISLLPPLGVAI |
| | | SVIIIFYCYRVNRQQKLSSTWETGKTRKLM |
| | | EFSEHCAIILEDDRSDISSTCANNINHNTE |
| | | LLPIELDTLVGKGRFAEVYKAKLKQNTSEQ |
| | | FETVAVKIFPYEEYASWKTEKDIFSDINLK |
| | | HENILQFLTAEERKTELGKQYWLITAFHAK |
| | | GNLQEYLTRHVISWEDLRKLGSSLARGIAH |
| | | LHSDHTPCGRPKMPIVHRDLKSSNILVKND |
| | | LTCCLCDFGLSLRLDPTLSVDDLANSGQVG |
| | | TARYMAPEVLESRMNLENVESFKQTDVYSM |
| | | ALVLWEMTSRCNAVGEVKDYEPPFGSKVRE |
| | | HPCVESMKDNVLRDRGRPEIPSFWLNHQGI |
| | | QMVCETLTECWDHDPEARLTAQCVAERFSE |
| | | LEHLDRLSGRSCSEEKIPEDGSLNTTK |

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs a functional domain (e.g., nuclease such as Cas, for example Cas9) to the target DNA via Watson-Crick base-pairing between the spacer on the crRNA and the protospacer on the target DNA next to the protospacer adjacent motif (PAM), an additional requirement for target recognition. Finally, Cas (e.g., Cas9) mediates cleavage of target DNA to create a double-stranded break within the protospacer. Activity of the CRISPR/Cas system comprises of three steps: (i) insertion of foreign DNA sequences into the CRISPR array to prevent future attacks, in a process called ‘adaptation’, (ii) expression of the relevant proteins, as well as expression and processing of the array, followed by (iii) RNA-mediated interference with the foreign nucleic acid. Thus, in the bacterial cell, several of the so-called ‘Cas’ proteins are involved with the natural function of the CRISPR/Cas system and serve roles in functions such as insertion of the foreign DNA etc.

Non-limiting examples of nucleases include meganucleases, TALENs and zinc finger nucleases. The nuclease may comprise heterologous DNA-binding and cleavage domains (e.g., zinc finger nucleases; meganuclease DNA-binding domains with heterologous cleavage domains) or, alternatively, the DNA-binding domain of a naturally-occurring nuclease may be altered to bind to a selected target site (e.g., a meganuclease that has been engineered to bind to site different than the cognate binding site).

### Expression of Chimeric Antigen Receptor or T-Cell Receptor

The engineered cells, in particular immune cells, such as T cells, NK cells and other immune cell types, may also be genetically engineered with vectors designed to express CARs or TCRs that redirect cytotoxicity toward tumor cells. CARs are molecules that combine antibody-based specificity for a target antigen (e.g., tumor antigen) with a T cell receptor-activating intracellular domain to generate a chimeric protein that exhibits a specific anti-tumor cellular immune activity.

The CARs contemplated herein comprise an extracellular domain that binds to a specific target antigen (also referred to as a binding domain or antigen-specific binding domain), a transmembrane domain and an intracellular signaling domain. A characteristic of CARs is their ability to redirect immune effector cell specificity, thereby triggering proliferation, cytokine production, phagocytosis or production of molecules that may mediate cell death of the target antigen expressing cell in a major histocompatibility (MHC) independent manner, exploiting the cell specific targeting abilities of monoclonal antibodies, soluble ligands or cell specific co-receptors.

In some embodiments, a CAR comprises an extracellular binding domain including but not limited to an antibody or antigen binding fragment thereof, a tethered ligand, or the extracellular domain of a co-receptor, that specifically binds a target antigen.

By way of non-limiting examples, target antigens may include: HPV oncoproteins, including HPV-16 E6 and HPV-16 E7, alpha folate receptor, 5T4, αᵥβ₆ integrin, BCMA, TACI, B7-H3, B7-H6, CAIX, CD19, CD20, CD22, CD28, CD30, CD33, CD44, CD44v6, CD44v7/8, CD70, CD79a, CD79b, CD123, CD137 (4-1BB), CD138, CD171, CEA, CSPG4, CLL-1, EGFR, EGFR family including ErbB2 (HERII), EGFRVIII, EGP2, EGP40, EPCAM, EphA2, EpCAM, FAP, fetal AchR, FRa, GD2, GD3, Glypican-3 (GPC3), HLA-A1⁺ MAGE1, HLA-A2⁺ MAGE1, HLAA3⁺ MAGE1, HLA-A1⁺ NY-ESO-1, HLA-A2⁺ NY-ESO-1, HLA-A3⁺ NY-ESO-1, IL-IIRα, IL-13Rα2, Lambda, Lewis-Y, Kappa, Mesothelin, Muc1, Muc16, NCAM, NKG2D Ligands, NYE-S0-1, PRAME, PSCA, PSMA, RORI, SSX, Survivin, TAG72, TEMs, FCRL5, DLL3, BAFFR and VEGFRII; one or more hinge domains or spacer domains; a transmembrane domain including, but not limited to, transmembrane domains from CD8a, CD4, CD45, PD-1, and CD152; one or more intracellular costimulatory signaling domains including but not limited to intracellular costimulatory signaling domains from CD28, CD54 (ICAM), CD134 (OX40), CD137 (41BB), CD152 (CTLA4), CD273 (PD-L2), CD274 (PD-L1), and CD278 (ICOS); and a primary signaling domain from CD3ζ or FcRγ. In some embodiments described herein, the CAR binds to a tumor antigen comprising CLL-1, CD19, CD20, CD28, CD137 (4-1BB), Glypican-3 (GPC3), PSCA or PSMA. In certain embodiments, the CAR binds CD19. In certain embodiments, the CAR binds CD20. In certain embodiments, the CAR includes a first scFv that binds CD19 and a second scFv that binds CD20. Example CD19- or CD20-binding sequences are provided in Table 4.

TABLE 4
Example Antigen-Binding Sequences

| Name | Sequence |
|---|---|
| Anti-CD20 v01 | SEQ ID NO: 24 |
| VH/VL | QVQLQQWGAGLLKPSETLSLTCAVYGGSFSGYYWSWIRQPPGKGLEWI |
| | GEIDHSGSTNYNPSLKSRVTISVDTSKNQFSLKLSSVTAADTAVYYCA |
| | RGGGSWYSNWFDPWGQGTMVTVSS |
| | SEQ ID NO: 25 |
| | DIQMTQSPSTLSASVGDRVTITCRASQSISSWLAWYQQKPGKAPKLLI |
| | YDASSLESGVPSRFSGSGSGTEFTLTISSLQPDDFATYYCQQDRSLPP |
| | TFGGGTKVEIK |
| Anti-CD20 v02 | SEQ ID NO: 26 |
| VH/VL | QVQLQQWGAGLLKPSETLSLTCAVYGGSFSGIHWNWIRQPPGKGLEWI |
| | GDIDTSGSTNYNPSLKSRVTISVDTSKNQFSLKLSSVTAADTAVYYCA |
| | RLGQESATYLGMDVWGQGTTVTVSS |
| | SEQ ID NO: 27 |
| | DIVMTQSPDSLAVSLGERATINCKSSQSVLYSSNNKNYLAWYQQKPGQ |
| | PPKLLIYWASTRESGVPDRFSGSGSGTDFTLTISSLQAEDVAVYYCQQ |
| | LYTYPFTFGGGTKVEIK |
| Anti-CD20 v03 | SEQ ID NO: 28 |
| VH/VL | QLQLQESGPGLVKPSETLSLTCTVSGGSISSSSYYWGWIRQPPGKGLE |
| | WIGSIYYSGSTYYNPSLKSRVTISVDTSKNQFSLKLSSVTAADTAVYY |
| | CARETDYSSGMGYGMDVWGQGTTVTVSS |
| | SEQ ID NO: 29 |
| | DIQMTQSPSSLSASVGDRVTITCRASQSINSYLNWYQQKPGKAPKLLI |
| | YAASSLQSGVPSRFSGSGSGTDFTLTISSLQPEDFATYYCQQSLADPF |
| | TFGGGTKVEIK |
| Anti-CD20 v04 | SEQ ID NO: 30 |
| VH/VL | QVQLVQSGAEVKKPGASVKVSCKASGYTFKEYGISWVRQAPGQGLEWM |
| | GWISAYSGHTYYAQKLQGRVTMTTDTSTSTAYMELRSLRSDDTAVYYC |
| | ARGPHYDDWSGFIIWFDPWGQGTLVTVSS |
| | SEQ ID NO: 31 |
| | DIQMTQSPSSLSASVGDRVTITCRASQSISSYLNWYQQKPGKAPKLLI |
| | YAASSLQSGVPSRFSGSGSGTDFTLTISSLQPEDFATYYCQQSYRFPP |
| | TFGQGTKVEIK |
| Anti-CD20 v05 | SEQ ID NO: 32 |
| VH/VL | QVQLQESGPGLVKPSETLSLTCTVSGGSISSPDHYWGWIRQPPGKGLE |
| | WIGSIYASGSTFYNPSLKSRVTISVDTSKNQFSLKLSSVTAADTAVYY |
| | CARETDYSSGMGYGMDVWGQGTTVTVSS |
| | SEQ ID NO: 33 |
| | DIQMTQSPSSLSASVGDRVTITCRASQSINSYLNWYQQKPGKAPKLLI |
| | YAASSLQSGVPSRFSGSGSGTDFTLTISSLQPEDFATYYCQQSLADPF |
| | TFGGGTKVEIK |
| Anti-CD20 v06 | SEQ ID NO: 34 |
| VH/VL | QITLKESGPTLVKPTQTLTLTCTFSGFSLDTEGVGVGWIRQPPGKALE |
| | WLALIYFNDQKRYSPSLKSRLTITKDTSKNQVVLTMTNMDPVDTAVYY |
| | CARDTGYSRWYYGMDVWGQGTTVTVSS |
| | SEQ ID NO: 35 |
| | DIQMTQSPSSVSASVGDRVTITCRASQGISSWLAWYQQKPGKAPKLLI |
| | YAASSLQSGVPSRFSGSGSGTDFTLTISSLQPEDFATYYCQQAYAYPI |
| | TFGGGTKVEIK |
| Anti-CD20 v07 | SEQ ID NO: 36 |
| VH/VL | QVQLQQWGAGLLKPSETLSLTCAVYGGSFEKYYWSWIRQPPGKGLEWI |
| | GEIYHSGLTNYNPSLKSRVTISVDTSKNQFSLKLSSVTAADTAVYYCA |
| | RVRYDSSDSYYYSYDYGMDVWGQGTTVTVSS |
| | SEQ ID NO: 37 |
| | DIVLTQSPDSLAVSLGERATINCKSSQSVLYSSNNKNYLAWYQQKPGQ |
| | PPKLLIYWASSRESGVPDRFSGSGSGTDFTLTISSLQAEDVAVYYCQQ |
| | SYSFPWTFGGGTKVEIK |
| Anti-CD20 v08 | SEQ ID NO: 38 |
| VH/VL | QVQLQQWGAGLLKPSETLSLTCAVYGGSFSRYVWSWIRQPPGKGLEWI |
| | GEIDSSGKTNYNPSLKSRVTISVDTSKNQFSLKLSSVTAADTAVYYCA |
| | RVRYDSSDSYYYSYDYGMDVWGQGTTVTVSS |
| | SEQ ID NO: 39 |
| | DIVLTQSPDSLAVSLGERATINCKSSQSVLYSSNNKNYLAWYQQKPGQ |
| | PPKLLIYWASSRESGVPDRFSGSGSGTDFTLTISSLQAEDVAVYYCQQ |
| | SYSFPWTFGGGTKVEIK |
| Anti-CD20 v09 | SEQ ID NO: 40 |
| VH/VL | QVQLQQWGAGLLKPSETLSLTCAVYGGSFSGYAWSWIRQPPGKGLEWI |
| | GEIDHRGFTNYNPSLKSRVTISVDTSKNQFSLKLSSVTAADTAVYYCA |
| | RVRYDSSDSYYYSYDYGMDVWGQGTTVTVSS |
| | SEQ ID NO: 41 |
| | DIVLTQSPDSLAVSLGERATINCKSSQSVLYSSNNKNYLAWYQQKPGQ |
| | PPKLLIYWASSRESGVPDRFSGSGSGTDFTLTISSLQAEDVAVYYCQQ |
| | SYSFPWTFGGGTKVEIK |
| Anti-CD20 v10 | SEQ ID NO: 42 |
| VH/VL | QVQLQQWGAGLLKPSETLSLTCAVYGGSFQKYYWSWIRQPPGKGLEWI |
| | GEIDTSGFTNYNPSLKSRVTISVDTSKNQFSLKLSSVTAADTAVYYCA |
| | RVGRYSYGYYITAFDIWGQGTTVTVSS |
| | SEQ ID NO: 43 |
| | DIVMTQSPDSLAVSLGERATINCKSSQSVLYSSNNKNYLAWYQQKPGQ |
| | PPKLLIYWASTRESGVPDRFSGSGSGTDFTLTISSLQAEDVAVYYCQQ |
| | HYSFPFTFGGGTKVEIK |
| Anti-CD19 | SEQ ID NO: 44 |
| VH/VL v01 | EVKLQESGPGLVAPSQSLSVTCTVSGVSLPDYGVSWIRQPPRKGLEWL |
| | GVIWGSETTYYNSALKSRLTIIKDNSKSQVFLKMNSLQTDDTAIYYCA |
| | KHYYYGGSYAMDYWGQGTSVTVSS |
| | SEQ ID NO: 45 |
| | DIQMTQTTSSLSASLGDRVTISCRASQDISKYLNWYQQKPDGTVKLLI |
| | YHTSRLHSGVPSRFSGSGSGTDYSLTISNLEQEDIATYFCQQGNTLPY |
| | TFGGGTKLEIT |
| Anti-CD19 | SEQ ID NO: 46 |
| VH/VL v02 | EVQLVESGGGLVQPGRSLRLSCTASGVSLPDYGVSWIRQPPGKGLEWI |
| | GVIWGSETTYYNSALKSRFTISRDNSKNTLYLQMNSLRAEDTAVYYCA |
| | KHYYYGGSYAMDYWGQGTLVTVSS |
| | SEQ ID NO: 47 |
| | DIQMTQSPSSLSASVGDRVTITCRASQDISKYLNWYQQKPDQAPKLLI |
| | KHTSRLHSGVPSRFSGSGSGTDYTLTISSLQPEDFATYYCQQGNTLPY |
| | TFGQGTKLEIK |
| Anti-CD19 | SEQ ID NO: 48 |
| scFv | DIQMTQTTSSLSASLGDRVTISCRASQDISKYLNWYQQKPDGTVKLLI |
| | YHTSRLHSGVPSRFSGSGSGTDYSLTISNLEQEDIATYFCQQGNTLPY |
| | TFGGGTKLEITGSTSGSGKPGSGEGSTKGEVKLQESGPGLVAPSQSLS |
| | VTCTVSGVSLPDYGVSWIRQPPRKGLEWLGVIWGSETTYYNSALKSRL |
| | TIIKDNSKSQVFLKMNSLQTDDTAIYYCAKHYYYGGSYAMDYWGQGTS |
| | VTVSS |
| Anti-CD20/ | SEQ ID NO: 49 |
| anti-CD19 | MLLLVTSLLLCELPHPAFLLIPDIQMTQTTSSLSASLGDRVTISCRASQDI |
| bicistronic | SKYLNWYQQKPDGTVKLLIYHTSRLHSGVPSRFSGSGSGTDYSLTISNL |
| CAR | EQEDIATYFCQQGNTLPYTFGGGTKLEITGGGGSGGGGSGGGGSEVKL |
| | QESGPGLVAPSQSLSVTCTVSGVSLPDYGVSWIRQPPRKGLEWLGVIWG |
| | SETTYYNSALKSRLTIIKDNSKSQVFLKMNSLQTDDTAIYYCAKHYYYG |
| | GSYAMDYWGQGTSVTVSSAAALDNEKSNGTIIHVKGKHLCPSPLFPGP |
| | SKPFWVLVVVGGVLACYSLLVTVAFIIFWVRSKRSRLLHSDYMNMTPR |
| | RPGPTRKHYQPYAPPRDFAAYRSRVKFSRSADAPAYQQGQNQLYNELN |
| | LGRREEYDVLDKRRGRDPEMGGKPRRKNPQEGLYNELQKDKMAEAYS |
| | EIGMKGERRRGKGHDGLYQGLSTATKDTYDALHMQALPPRRAKRSGS |
| | GEGRGSLLTCGDVEENPGPMALPVTALLLPLALLLHAARPQLQLQESGP |
| | GLVKPSETLSLTCTVSGGSISSSSYYWGWIRQPPGKGLEWIGSIYYSGST |
| | YYNPSLKSRVTISVDTSKNQFSLKLSSVTAADTAVYYCARETDYSSGM |
| | GYGMDVWGQGTTVTVSSGSTSGSGKPGSGEGSTKGDIQMTQSPSSLSA |
| | SVGDRVTITCRASQSINSYLNWYQQKPGKAPKLLIYAASSLQSGVPSRFS |
| | GSGSGTDFTLTISSLQPEDFATYYCQQSLADPFTFGGGTKVEIKAAAFVP |
| | VFLPAKPTTTPAPRPPTPAPTIASQPLSLRPEACRPAAGGAVHTRGLDFA |
| | CDIYIWAPLAGTCGVLLLSLVITLYCNHRNRFSVVKRGRKKLLYIFKQPF |
| | MRPVQTTQEEDGCSCRFPEEEEGGCELRVKFSRSADAPAYQQGQNQLY |
| | NELNLGRREEYDVLDKRRGRDPEMGGKPRRKNPQEGLYNELQKDKM |
| | AEAYSEIGMKGERRRGKGHDGLYQGLSTATKDTYDALHMQALPPR |
| Anti-CD20/ | SEQ ID NO: 50 |
| anti-CD19 | MLLLVTSLLLCELPHPAFLLIPDIQMTQSPSSLSASVGDRVTITCRASQSI |
| bispecific | NSYLNWYQQKPGKAPKLLIYAASSLQSGVPSRFSGSGSGTDFTLTISSLQ |
| CAR | PEDFATYYCQQSLADPFTFGGGTKVEIKGGGGSGKPGSGEGGSQLQLQ |
| | ESGPGLVKPSETLSLTCTVSGGSISSSSYYWGWIRQPPGKGLEWIGSIYY |
| | SGSTYYNPSLKSRVTISVDTSKNQFSLKLSSVTAADTAVYYCARETDYS |
| | SGMGYGMDVWGQGTTVTVSSGGGGSGKPGSDIQMTQSPSSLSASVGD |
| | RVTITCRASQDISKYLNWYQQKPDQAPKLLIKHTSRLHSGVPSRFSGSGS |
| | GTDYTLTISSLQPEDFATYYCQQGNTLPYTFGQGTKLEIKGGGGSGGGG |
| | SGGGGSEVQLVESGGGLVQPGRSLRLSCTASGVSLPDYGVSWIRQPPGK |
| | GLEWIGVIWGSETTYYNSALKSRFTISRDNSKNTLYLQMNSLRAEDTAV |
| | YYCAKHYYYGGSYAMDYWGQGTLVTVSSAAALDNEKSNGTIIHVKG |
| | KHLCPSPLFPGPSKPFWVLVVVGGVLACYSLLVTVAFIIFWVRSKRSRL |
| | LHSDYMNMTPRRPGPTRKHYQPYAPPRDFAAYRSRVKFSRSADAPAYQ |
| | QGQNQLYNELNLGRREEYDVLDKRRGRDPEMGGKPRRKNPQEGLYNE |
| | LQKDKMAEAYSEIGMKGERRRGKGHDGLYQGLSTATKDTYDALHMQ |
| | ALPPR |

A hinge may be derived from a natural source or from a synthetic source. In some embodiments, an antigen binding system of the present disclosure may comprise a hinge that is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA1-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof. In certain embodiments, a CAR does not comprise a CD28 hinge.

A transmembrane domain may be derived either from a natural or from a synthetic source. Where the source is natural, a domain may be derived from any membrane-bound or transmembrane protein. Exemplary transmembrane domains may be derived from (e.g., may comprise at least a transmembrane domain of) an alpha, beta or zeta chain of a T cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof. In some embodiments, a transmembrane domain is synthetic (and can, e.g., comprise predominantly hydrophobic residues such as leucine and valine). In some embodiments, a triplet of phenylalanine, tryptophan and valine are comprised at each end of a synthetic transmembrane domain. In some embodiments, a transmembrane domain is directly linked or connected to a cytoplasmic domain. In some embodiments, a short oligo- or polypeptide linker (e.g., between 2 and 10 amino acids in length) may form a linkage between a transmembrane domain and an intracellular domain. In some embodiments, a linker is a glycine-serine doublet.

In some embodiments, a signaling domain and/or activation domain comprises an immunoreceptor tyrosine-based activation motif (ITAM). Non-limiting examples of ITAM containing cytoplasmic signaling sequences comprise those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2: a002485 (2010); Smith-Garvin et al., Annu. Rev. Immunol. 27:591-619 (2009)).

A CAR may comprise a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), OX40, CD30, CD40, PD-1, ICOS, lymphocyte function-associated antigen-1 (LFA-1), MYD88, CD2, CD7, LIGHT, NKG2C, B7-H3, and a ligand that binds with CD83.

In certain embodiments, the CARs contemplated herein may comprise linker residues between the various domains, e.g., between VH and VL domains, added for appropriate spacing conformation of the molecule. CARs contemplated herein, may comprise one, two, three, four, or five or more linkers. In some embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In some embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. In some embodiments, a signaling domain and/or activation domain comprises an immunoreceptor tyrosine-based activation motif (ITAM). Examples of ITAM containing cytoplasmic signaling sequences comprise those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2: a002485 (2010); Smith-Garvin et al., Annu. Rev. Immunol. 27:591-619 (2009)). In certain embodiments, suitable signaling domains comprise, without limitation, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

Effector functions of the T cell, for example, may be cytolytic activity or activity including the secretion of a cytokine. The effector function of a T cell may be stimulated by an effector function signal transduced by the intracellular signaling domain of a CAR. While usually the entire intracellular signaling domain is employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

In some embodiments, the cell may be engineered to express an exogenous T cell receptor (TCR). Libraries of TCRs may be screened for their selectivity to target antigens. In this manner, natural TCRs, which have a high avidity and reactivity toward target antigens may be selected, cloned, and subsequently introduced into a population of T cells used for adoptive immunotherapy.

In some embodiments described herein, T cells are modified by introducing a polynucleotide encoding subunit of a TCR that may form TCRs that confer specificity to T cells for tumor cells expressing a target antigen. In some embodiments, the subunits have one or more amino acid substitutions, deletions, insertions, or modifications compared to the naturally occurring subunit, so long as the subunits retain the ability to form TCRs conferring upon transfected T cells the ability to home to target cells, and participate in immunologically-relevant cytokine signaling. The TCRs may also bind target cells displaying the relevant tumor-associated peptide with high avidity, and optionally mediate efficient killing of target cells presenting the relevant peptide in vivo.

The nucleic acids encoding TCRs may be isolated from their natural context in a (naturally-occurring) chromosome of a T cell and may be incorporated into suitable vectors as described elsewhere herein. Both the nucleic acids and the vectors comprising them may be transferred into a cell, which cell may be a T cell. The modified T cells are then able to express one or more chains of a TCR (and in some aspects two chains) encoded by the transduced nucleic acid or nucleic acids. In some embodiments, the TCR is an exogenous TCR because it is introduced into T cells that do not normally express the introduced TCR. An aspect of the TCRs is that it has high avidity for a tumor antigen presented by a major histocompatibility complex (MHC) or similar immunological component. In contrast to TCRs, CARs are engineered to bind target antigens in an MHC independent manner.

The protein encoded by the nucleic acids described herein may be expressed with additional polypeptides attached to the amino-terminal or carboxyl-terminal portion of the α-chain or the β-chain of a TCR so long as the attached additional polypeptide does not interfere with the ability of the α-chain or the β-chain to form a functional T cell receptor and the MHC dependent antigen recognition.

Antigens that are recognized by the TCRs contemplated herein include, but are not limited to, cancer antigens, including antigens on both hematological cancers and solid tumors and viral induced cancers. Other illustrative antigens include, but are not limited to, HPV oncoproteins, including HPV-16 E6 and HPV-16 E7, alpha folate receptor, 5T4, αᵥβ₆ integrin, BCMA, TACI, B7-H3, B7-H6, CAIX, CD19, CD20, CD22, CD28, CD30, CD33, CD44, CD44v6, CD44v7/8, CD70, CD79a, CD79b, CD123, CD137 (4-1BB), CD138, CD171, CEA, CSPG4, CLL-1, EGFR, EGFR family including ErbB2 (HERII), EGFRVIII, EGP2, EGP40, EPCAM, EphA2, EpCAM, FAP, fetal AchR, FRa, GD2, GD3, Glypican-3 (GPC3), HLA-A1⁺ MAGE1, HLA-A2⁺ MAGE1, HLAA3⁺ MAGE1, HLA-A1⁺NY-ES0-1, HLA-A2⁺NY-ESO-1, HLA-A3⁺NY-ESO-1, IL-11Rα, IL-13Rα2, Lambda, Lewis-Y, Kappa, Mesothelin, Muc1, Muc16, NCAM, NKG2D Ligands, NY-ES0-1, PRAME, PSCA, PSMA, RORI, SSX, Survivin, TAG72, TEMs, DLL3, FCRL5, BAFFR, and VEGFRII.

In some embodiments, the polynucleotide that encodes the CAR or TCR is introduced to the cell after the cell is engineered to reduce the CD58, RFX5, ICAM1, TGFBR2 and/or FAS expression or activity. In a preferred embodiment, the polynucleotide that encodes the CAR or TCR is introduced to the cell before the cell is engineered to reduce the CD58, RFX5, ICAM1, TGFBR2 and/or FAS expression or activity. In some embodiments, the two rounds of engineering are carried out at least one day apart (not on the same day or within 24 hours). Method of Treatment

The cells, e.g., allogeneic cells, of the present disclosure may be used for treating various diseases and conditions, in particular cancer and/or autoimmune disease. In some embodiments, the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer. systemic lupus erythematosus, rheumatoid arthritis, myositis, myasthenia gravis, multiple sclerosis, Sjogren's syndrome, psoriasis, and inflammatory bowel disease.

In some embodiments, the cells of the present disclosure may be used to treat myeloid diseases including but not limited to acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN), or combinations thereof. Additional diseases include inflammatory and/or autoimmune diseases such as rheumatoid arthritis, psoriasis, allergies, asthma, Crohn's disease, IBD, IBS, fibromyalga, mastocytosis, lupus, and Celiac disease.

In some embodiments, the cells of the present disclosure may be used to treat cancer and/or autoimmune diseases that arise from B cells, e.g., B-cell lymphomas. In some embodiments, cells of the present disclosure may be used to treat diffuse large B-cell lymphoma (DLBCL) not otherwise specified, primary mediastinal large B-cell lymphoma, high grade B-cell lymphoma, and DLBCL arising from follicular lymphoma.

In a third aspect, the present disclosure provides a method of treating cancer and/or auto immune disease in a patient in need thereof comprising administering a therapeutically effective amount of a cell of the present disclosure. In some embodiments, the cell is not originally derived from the patient.

The quantity and frequency of administration will be determined by such factors as the condition of the patient, and the type and severity of the patient's disease, although appropriate dosages may be determined by clinical trials. In some embodiments, the cancer and/or autoimmune disease is characterized with the expression of an antigen targeted by the CAR or TCR molecule, such as CD19 and/or CD20. In some embodiments, the CAR recognizes CD19, CD20, CD22, BCMA, TACI, EGFRVIII, IL13RA, GPC3, GPC2, and/or CD38. In some embodiments, the cancer is characterized with the expression CD19. In some embodiments, the cancer is characterized with the expression CD20. In some embodiments, the cancer is characterized with the expression CD19 and CD20.

In other embodiments, methods comprising administering a therapeutically effective amount of modified T cells contemplated herein or a composition comprising the same, to a patient in need thereof, alone or in combination with one or more therapeutic agents, are provided. In certain embodiments, the cells of the disclosure are used in the treatment of patients at risk for developing a cancer and/or autoimmune disease. Thus, the present disclosure provides methods for the treatment or prevention of a cancer and/or autoimmune disease comprising administering to a patient in need thereof, a therapeutically effective amount of the modified T cells of the disclosure.

One of ordinary skill in the art would recognize that multiple administrations of the compositions of the disclosure may be required to affect the desired therapy. For example, a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In some embodiments, a patient in need thereof is administered an effective amount of a composition to increase a cellular immune response to a cancer and/or autoimmune disease in the patient. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

The methods for administering the cell compositions described herein includes any method which is effective to result in reintroduction of ex vivo genetically modified immune effector cells that either directly express an TCR or CAR in the patient or on reintroduction of the genetically modified progenitors of immune effector cells that on introduction into a patient differentiate into mature immune effector cells that express the TCR or CAR.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield similar results.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control. The contents of all references cited throughout this application are expressly incorporated herein by reference.

### Embodiments

Embodiment 1: An isolated immune cell engineered to have CD58 expression or activity that is at least 10% lower as compared to a corresponding non-engineered immune cell and engineered to have FAS expression or activity that is at least 10% lower as compared to a corresponding non-engineered immune cell.

Embodiment 2: The cell of embodiment 1, further engineered to have ICAM1 expression or activity that is at least 10% lower as compared to a corresponding non-engineered immune cell.

Embodiment 3: The cell of any one of the proceeding embodiments, further engineered to have RFX5 expression or activity that is at least 10% lower as compared to a corresponding non-engineered immune cell.

Embodiment 4: The cell of any one of the proceeding embodiments, further engineered to have TGFBR2 expression or activity that is at least 10% lower as compared to a corresponding non-engineered immune cell.

Embodiment 5: The cell of any one of embodiments 1-4, wherein the expression of CD58 is reduced by at least 10%.

Embodiment 6: The cell of any one of embodiments 1-4, wherein the activity of CD58 is reduced by at least 10%.

Embodiment 7: The cell of any one of embodiments 1-4, wherein the expression or activity of CD58 is reduced by at least 75%.

Embodiment 8: The cell of any one of embodiments 1-4, wherein the expression of CD58 is reduced by at least 75%.

Embodiment 9: The cell of any one of embodiments 1-4, wherein the activity of CD58 is reduced by at least 75%.

Embodiment 10: The cell of any one of embodiments 1-4, wherein the expression or activity of CD58 is eliminated.

Embodiment 11: The cell of any one of embodiments 1-4, wherein the expression of CD58 is eliminated.

Embodiment 12: The cell of any one of embodiments 1-4, wherein the activity of CD58 is eliminated.

Embodiment 13: The cell of any one of embodiments 1-12, wherein the expression of FAS is reduced by at least 10%.

Embodiment 14: The cell of any one of embodiments 1-12, wherein the activity of FAS is reduced by at least 10%.

Embodiment 15: The cell of any one of embodiments 1-12, wherein the expression or activity of FAS is reduced by at least 75%.

Embodiment 16: The cell of any one of embodiments 1-12, wherein the expression of FAS is reduced by at least 75%.

Embodiment 17: The cell of any one of embodiments 1-12, wherein the activity of FAS is reduced by at least 75%.

Embodiment 18: The cell of any one of embodiments 1-12, wherein the expression or activity of FAS is eliminated.

Embodiment 19: The cell of any one of embodiments 1-12, wherein the expression of FAS is eliminated.

Embodiment 20: The cell of any one of embodiments 1-12, wherein the activity of FAS is eliminated.

Embodiment 21: The cell of any one of embodiments 2-20, wherein the expression of ICAM1 is reduced by at least 10%.

Embodiment 22: The cell of any one of embodiments 2-20, wherein the activity of ICAM1 is reduced by at least 10%.

Embodiment 23: The cell of any one of embodiments 2-20, wherein the expression or activity of ICAM1 is reduced by at least 75%.

Embodiment 24: The cell of any one of embodiments 2-20, wherein the expression of ICAM1 is reduced by at least 75%.

Embodiment 25: The cell of any one of embodiments 2-20, wherein the activity of ICAM1 is reduced by at least 75%.

Embodiment 26: The cell of any one of embodiments 2-20, wherein the expression or activity of FAS is eliminated.

Embodiment 27: The cell of any one of embodiments 2-20, wherein the expression of FAS is eliminated.

Embodiment 28: The cell of any one of embodiments 2-20, wherein the activity of FAS is eliminated.

Embodiment 29: The cell of any one of embodiments 3-28, wherein the cell is engineered to have RFX5 expression or activity that is at least 10% lower as compared to the corresponding non-engineered immune cell.

Embodiment 30: The cell of any one of embodiments 3-28, wherein the expression of RFX5 is reduced by at least 10%.

Embodiment 31: The cell of any one of embodiments 3-28, wherein the activity of RFX5 is reduced by at least 10%.

Embodiment 32: The cell of any one of embodiments 3-28, wherein the expression or activity of RFX5 is reduced by at least 75%.

Embodiment 33: The cell of any one of embodiments 3-28, wherein the expression of RFX5 is reduced by at least 75%.

Embodiment 34: The cell of any one of embodiments 3-28, wherein the activity of RFX5 is reduced by at least 75%.

Embodiment 35: The cell of any one of embodiments 3-28, wherein the expression or activity of RFX5 is eliminated.

Embodiment 36: The cell of any one of embodiments 3-28, wherein the expression of RFX5 is eliminated.

Embodiment 37: The cell of any one of embodiments 3-28, wherein the activity of RFX5 is eliminated.

Embodiment 38: The cell of any one of embodiments 4-37, wherein the cell is engineered to have TGFBR2 expression or activity that is at least 10% lower as compared to the corresponding non-engineered immune cell.

Embodiment 39: The cell of any one of embodiments 3-37, wherein the expression of TGFBR2 is reduced by at least 10%.

Embodiment 40: The cell of any one of embodiments 3-37, wherein the activity of TGFBR2 is reduced by at least 10%.

Embodiment 41: The cell of any one of embodiments 3-37, wherein the expression or activity of TGFBR2 is reduced by at least 75%.

Embodiment 42: The cell of any one of embodiments 3-37, wherein the expression of TGFBR2 is reduced by at least 75%.

Embodiment 43: The cell of any one of embodiments 3-37, wherein the activity of TGFBR2 is reduced by at least 75%.

Embodiment 44: The cell of any one of embodiments 3-37, wherein the expression or activity of TGFBR2 is eliminated.

Embodiment 45: The cell of any one of embodiments 3-37, wherein the expression of TGFBR2 is eliminated.

Embodiment 46: The cell of any one of embodiments 3-37, wherein the activity of TGFBR2 is eliminated.

Embodiment 47: An isolated immune cell engineered to have CD58 expression or activity that is at least 10% lower as compared to a corresponding non-engineered immune cell and engineered to have ICAM1 expression or activity that is at least 10% lower as compared to a corresponding non-engineered immune cell.

Embodiment 48: The cell of embodiment 47, further engineered to have RFX5 expression or activity that is at least 10% lower as compared to a corresponding non-engineered immune cell.

Embodiment 49: The cell of embodiment 47, further engineered to have TGFBR2 expression or activity that is at least 10% lower as compared to a corresponding non-engineered immune cell.

Embodiment 50: The cell of any of embodiments 47-49, wherein the expression of CD58 is reduced by at least 10%.

Embodiment 51: The cell of any of embodiments 47-49, wherein the activity of CD58 is reduced by at least 10%.

Embodiment 52: The cell of any of embodiments 47-49, wherein the expression or activity of CD58 is reduced by at least 75%.

Embodiment 53: The cell of any of embodiments 47-49, wherein the expression of CD58 is reduced by at least 75%.

Embodiment 54: The cell of any of embodiments 47-49, wherein the activity of CD58 is reduced by at least 75%.

Embodiment 55: The cell of any of embodiments 47-49, wherein the expression or activity of CD58 is eliminated.

Embodiment 56: The cell of any of embodiments 47-49, wherein the expression of CD58 is eliminated.

Embodiment 57: The cell of any of embodiments 47-49, wherein the activity of CD58 is eliminated.

Embodiment 58: The cell of any one of embodiments 47-57, wherein the expression of ICAM1 is reduced by at least 10%.

Embodiment 59: The cell of any one of embodiments 47-57, wherein the activity of ICAM1 is reduced by at least 10%.

Embodiment 60: The cell of any one of embodiments 47-57, wherein the expression or activity of ICAM1 is reduced by at least 75%.

Embodiment 61: The cell of any one of embodiments 47-57, wherein the expression of ICAM1 is reduced by at least 75%.

Embodiment 62: The cell of any one of embodiments 47-57, wherein the activity of ICAM1 is reduced by at least 75%.

Embodiment 63: The cell of any one of embodiments 47-57, wherein the expression or activity of ICAM1 is eliminated.

Embodiment 64: The cell of any one of embodiments 47-57, wherein the expression of ICAM1 is eliminated.

Embodiment 65: The cell of any one of embodiments 47-57, wherein the activity of ICAM1 is eliminated.

Embodiment 66: The cell of any one of embodiments 47-65, wherein the cell is engineered to have RFX5 expression or activity that is at least 10% lower as compared to the corresponding non-engineered immune cell.

Embodiment 67: The cell of any one of embodiments 47-65, wherein the expression of RFX5 is reduced by at least 10%.

Embodiment 68: The cell of any one of embodiments 47-65, wherein the activity of RFX5 is reduced by at least 10%.

Embodiment 69: The cell of any one of embodiments 47-65, wherein the expression or activity of RFX5 is reduced by at least 75%.

Embodiment 70: The cell of any one of embodiments 47-65, wherein the expression of RFX5 is reduced by at least 75%.

Embodiment 71: The cell of any one of embodiments 47-65, wherein the activity of RFX5 is reduced by at least 75%.

Embodiment 72: The cell of any one of embodiments 47-65, wherein the expression or activity of RFX5 is eliminated.

Embodiment 73: The cell of any one of embodiments 47-65, wherein the expression of RFX5 is eliminated.

Embodiment 74: The cell of any one of embodiments 47-65, wherein the activity of RFF5 is eliminated.

Embodiment 75: The cell of any one of embodiments 49-74, wherein the cell is engineered to have TGFBR2 expression or activity that is at least 10% lower as compared to the corresponding non-engineered immune cell.

Embodiment 76: The cell of any one of embodiments 49-74, wherein the expression of TGFBR2 is reduced by at least 10%.

Embodiment 77: The cell of any one of embodiments 49-74, wherein the activity of TGFBR2 is reduced by at least 10%.

Embodiment 78: The cell of any one of embodiments 49-74, wherein the expression or activity of TGFBR2 is reduced by at least 75%.

Embodiment 79: The cell of any one of embodiments 49-74, wherein the expression of TGFBR2 is reduced by at least 75%.

Embodiment 80: The cell of any one of embodiments 49-74, wherein the activity of TGFBR2 is reduced by at least 75%.

Embodiment 81: The cell of any one of embodiments 49-74, wherein the expression or activity of TGFBR2 is eliminated.

Embodiment 82: The cell of any one of embodiments 49-74, wherein the expression of TGFBR2 is eliminated.

Embodiment 83: The cell of any one of embodiments 49-74, wherein the activity of TGFBR2 is eliminated.

Embodiment 84: The cell of any one of embodiments 1-83, wherein the cell is a T cell or a natural killer (NK) cell.

Embodiment 85: The cell of any one of embodiments 1-83, wherein the cell is a human cell.

Embodiment 86: The cell of any one of embodiments 1-85, wherein the cell comprises an exogenous polynucleotide encoding a chimeric antigen receptor (CAR) or a T-cell receptor (TCR); wherein the TCR gene is one of TRAC, TRBC, CD3E, CD3G, CD3D, or CD3Z.

Embodiment 87: The cell of embodiment 66, wherein the CAR recognizes at least one of CD19, CD20, CD22, BCMA, TACI, EGFRVIII, IL13RA, GPC3, GPC2, and CD38.

Embodiment 88: The cell of embodiment 87, wherein the CAR recognizes CD19 and/or CD20.

Embodiment 89: The cell of any one of embodiments 1-88, wherein the expression or activity of endogenous CD3E is also reduced in the cell.

Embodiment 90: The cell of any one of embodiments 1-89, wherein the endogenous B2M (Beta-2-microglobulin) gene is not engineered, or wherein the cell has normal activity of B2M.

Embodiment 91: The cell of any one of embodiments 1-90, wherein the cell has normal activity of MHC Class I.

Embodiment 92: The cell of any one of embodiments 1-91, wherein the reduction in CD58 expression or activity is achieved by: (a) editing of the endogenous gene encoding CD58, (b) expression of an inhibitory RNA, or (c) an inhibitor, preferably an antibody.

Embodiment 93: The cell of embodiment 92, wherein the reduction in CD58 expression or activity is achieved by editing of an endogenous gene encoding CD58.

Embodiment 94: The cell of embodiment 93, wherein the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editing (ABE8 or BE4MAX).

Embodiment 95: The cell of embodiment 94, wherein the editing is by ABE8.

Embodiment 96: The cell of embodiment 94, wherein the editing is by BE4MAX.

Embodiment 97: The cell of any one of embodiments 1-96, wherein, upon administration to a patient, the cell is characterized by reduced activity in inducing graft-versus-host disease (GVHD) or host rejection.

Embodiment 98: The cell of any one of embodiments 1-97, wherein, upon administration to a patient, the cell is characterized by reduced killing by MHC-mismatched CD8⁺ T cells and/or NK cells.

Embodiment 99: A method for preparing an allogeneic immune cell with reduced activity in inducing graft-versus-host disease (GVHD) or host rejection, comprising reducing, in the cell, the expression or activity of CD58 by at least 10% as compared to a corresponding non-engineered immune cell, further comprising reducing, in the cell, the expression or activity of FAS by at least 10% as compared to a corresponding non-engineered cell.

Embodiment 100: The method of embodiment 99, wherein the CD58 expression is reduced by at least 10%.

Embodiment 101: The method of embodiment 99, wherein the CD58 activity is reduced by at least 10%.

Embodiment 102: The method of embodiment 99, wherein the expression or activity of CD58 is reduced by at least 75%.

Embodiment 103: The method of embodiment 99, wherein the CD58 expression is reduced by at least 75%.

Embodiment 104: The method of embodiment 99, wherein the CD58 activity is reduced by at least 75%.

Embodiment 105: The method of embodiment 99, wherein the expression or activity of CD58 is eliminated.

Embodiment 106: The method of embodiment 99, wherein the expression of CD58 is eliminated.

Embodiment 107: The method of embodiment 99, wherein the activity of CD58 is eliminated.

Embodiment 108: The method of any one of embodiments 99-107, wherein the FAS expression is reduced by at least 10%.

Embodiment 109: The method of any one of embodiments 99-107, wherein the FAS activity is reduced by at least 10%.

Embodiment 110: The method of any one of embodiments 99-107, wherein the expression or activity of FAS is reduced by at least 75%.

Embodiment 111: The method of any one of embodiments 99-107, wherein the FAS expression is reduced by at least 75%.

Embodiment 112: The method of any one of embodiments 99-107, wherein the FAS activity is reduced by at least 75%.

Embodiment 113: The method of any one of embodiments 99-107, wherein the expression or activity of FAS is eliminated.

Embodiment 114: The method of any one of embodiments 99-107, wherein the expression of FAS is eliminated.

Embodiment 115: The method of any one of embodiments 99-107, wherein the activity of FAS is eliminated.

Embodiment 116: The method of any one of embodiments 99-115, comprising reducing, in the cell, the expression or activity of ICAM1 by at least 10% as compared to a corresponding non-engineered immune cell.

Embodiment 117: The method embodiment 116, wherein the expression of ICAM1 is reduced by at least 10%

Embodiment 118: The method of embodiment 116, wherein the activity of ICAM1 is reduced by at least 10%.

Embodiment 119: The method of embodiment 116, wherein the expression or activity of ICAM1 is reduced by at least 75%.

Embodiment 120: The method of embodiment 116, wherein the expression of ICAM1 is reduced at least by 75%.

Embodiment 121: The method of embodiment 116, wherein the activity of ICAM1 is reduced by at least 75%.

Embodiment 122: The method of embodiment 116, wherein the expression or activity of ICAM1 is eliminated.

Embodiment 123: The method of embodiment 116, wherein the expression of ICAM1 is eliminated.

Embodiment 124: The method of embodiment 116, wherein the activity of ICAM1 is eliminated.

Embodiment 125: The method of any one of embodiments 99-124, comprising reducing, in the cell, the expression or activity of RFX5 by at least 10% as compared to a corresponding non-engineered immune cell.

Embodiment 126: The method of embodiment 125, wherein the expression of RFX5 is reduced by at least 10%.

Embodiment 127: The method of embodiment 125, wherein the activity of RFX5 is reduced by at least 10%.

Embodiment 128: The method of embodiment 125, wherein the expression or activity of RFX5 is reduced by at least 75%.

Embodiment 129: The method of embodiment 125, wherein the expression of RFX5 is reduced at least by 75%.

Embodiment 130: The method of embodiment 125, wherein the activity of RFX5 is reduced by at least 75%.

Embodiment 131: The method of embodiment 125, wherein the expression or activity of RFX5 is eliminated.

Embodiment 132: The method of embodiment 125, wherein the expression of RFX5 is eliminated.

Embodiment 133: The method of embodiment 125, wherein the activity of RFX5 is eliminated.

Embodiment 134: A method for preparing an allogeneic immune cell with reduced activity in inducing graft-versus-host disease (GVHD) or host rejection, comprising reducing, in the cell, the expression or activity of CD58 by at least 10% as compared to a corresponding non-engineered immune cell, further comprising reducing, in the cell, the expression or activity of ICAM1 by at least 10% as compared to a corresponding non-engineered cell.

Embodiment 135: The method of embodiment 134, further engineered to have RFX5 expression or activity that is at least 10% lower as compared to a corresponding non-engineered immune cell.

Embodiment 136: The method of embodiment 134 or 135, further engineered to have TGFBR2 expression or activity that is at least 10% lower as compared to a corresponding non-engineered immune cell.

Embodiment 137: The method of any of embodiments 134-136, wherein the expression of CD58 is reduced by at least 10%.

Embodiment 138: The method of any of embodiments 134-136, wherein the activity of CD58 is reduced by at least 10%.

Embodiment 139: The method of any of embodiments 134-136, wherein the expression or activity of CD58 is reduced by at least 75%.

Embodiment 140: The method of any of embodiments 134-136, wherein the expression of CD58 is reduced by at least 75%.

Embodiment 141: The method of any of embodiments 134-136, wherein the activity of CD58 is reduced by at least 75%.

Embodiment 142: The method of any of embodiments 134-136, wherein the expression or activity of CD58 is eliminated.

Embodiment 143: The method of any of embodiments 134-136, wherein the expression of CD58 is eliminated.

Embodiment 144: The method of any of embodiments 134-136, wherein the activity of CD58 is eliminated.

Embodiment 145: The method of any one of embodiments 134-144, wherein the expression of ICAM1 is reduced by at least 10%.

Embodiment 146: The method of any one of embodiments 134-144, wherein the activity of ICAM1 is reduced by at least 10%.

Embodiment 147: The method of any one of embodiments 134-144, wherein the expression or activity of ICAM1 is reduced by at least 75%.

Embodiment 148: The method of any one of embodiments 134-144, wherein the expression of ICAM1 is reduced by at least 75%.

Embodiment 149: The method of any one of embodiments 134-144, wherein the activity of ICAM1 is reduced by at least 75%.

Embodiment 150: The method of any one of embodiments 134-144, wherein the expression or activity of ICAM1 is eliminated.

Embodiment 151: The method of any one of embodiments 134-144, wherein the expression of ICAM1 is eliminated.

Embodiment 152: The method of any one of embodiments 134-144, wherein the activity of ICAM1 is eliminated.

Embodiment 153: The method of any one of embodiments 135-152, wherein the cell is engineered to have RFX5 expression or activity that is at least 10% lower as compared to the corresponding non-engineered immune cell.

Embodiment 154: The method of any one of embodiments 135-152, wherein the expression of RFX5 is reduced by at least 10%.

Embodiment 155: The method of any one of embodiments 135-152, wherein the activity of RFX5 is reduced by at least 10%.

Embodiment 156: The method of any one of embodiments 135-152, wherein the expression or activity of RFX5 is reduced by at least 75%.

Embodiment 157: The method of any one of embodiments 135-152, wherein the expression of RFX5 is reduced by at least 75%.

Embodiment 158: The method of any one of embodiments 135-152, wherein the activity of RFX5 is reduced by at least 75%.

Embodiment 159: The method of any one of embodiments 135-152, wherein the expression or activity of RFX5 is eliminated.

Embodiment 160: The method of any one of embodiments 135-152, wherein the expression of RFX5 is eliminated.

Embodiment 161: The method of any of embodiments 136-160, wherein the expression of TGFBR2 is reduced by at least 10%.

Embodiment 162: The method of any of embodiments 136-160, wherein the activity of TGFBR2 is reduced by at least 10%.

Embodiment 163: The method of any of embodiments 136-160, wherein the expression or activity of TGFBR2 is reduced by at least 75%.

Embodiment 164: The method of any of embodiments 136-160, wherein the expression of TGFBR2 is reduced by at least 75%.

Embodiment 165: The method of any of embodiments 136-160, wherein the activity of TGFBR2 is reduced by at least 75%.

Embodiment 166: The method of any of embodiments 136-160, wherein the expression or activity of TGFBR2 is eliminated.

Embodiment 167: The method of any of embodiments 136-160, wherein the expression of TGFBR2 is eliminated.

Embodiment 168: The method of any one of embodiments 134-167, wherein the cell is a T cell or a natural killer (NK) cell.

Embodiment 169: The method of any one of embodiments 134-168, wherein the cell is a human cell.

Embodiment 170: The method of any one of embodiments 134-169, wherein the method further comprises introducing into the cell an exogenous polynucleotide encoding a chimeric antigen receptor (CAR) or a T-cell receptor (TCR); wherein the TCR gene is one of TRAC, TRBC, CD3E, CD3G, CD3D, or CD3Z.

Embodiment 171: The method of embodiment 170, wherein the CAR recognizes at least one of CD19, CD20, CD22, BCMA, TACI, EGFRVIII, IL13RA, GPC3, GPC2, and CD38.

Embodiment 172: The method of embodiment 171, wherein the CAR recognizes CD19 and/or CD20.

Embodiment 173: The method of any one of embodiments 134-172, wherein the method further comprises reducing the expression or activity of CD3E in the cell.

Embodiment 174: The method of any one of embodiments 134-170, wherein the endogenous B2M (Beta-2-microglobulin) gene in the cell is not engineered, or wherein the cell has normal activity of B2M.

Embodiment 175: The method of any one of embodiments 134-170, wherein the cell has normal activity of MHC Class I.

Embodiment 176: The method of any one of embodiments 134-170, wherein the reduction in CD58 expression or activity is achieved by: (a) editing of the endogenous gene encoding CD58, (b) expression of an inhibitory RNA, or (c) an inhibitor, preferably an antibody.

Embodiment 177: The method of embodiment 176, wherein the reduction in CD58 expression or activity is achieved by editing of the endogenous gene encoding CD58.

Embodiment 178: The method of embodiment 177, wherein the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editing (ABE8 or BE4MAX).

Embodiment 179: The method of embodiment 178, wherein the editing is by ABE8.

Embodiment 180: The method of embodiment 178, wherein the editing is by BE4MAX.

Embodiment 181: The method of any of embodiments 170-180, wherein the CAR or TCR is introduced into the cell by transduction with a lentiviral vector.

Embodiment 182: The method of any one of embodiments 170-181, wherein the CAR or TCR is introduced into the cell prior to editing of the gene encoding CD58.

Embodiment 183: A method for treating cancer and/or autoimmune diseases in a patient in need thereof, comprising administering to the patient a therapeutically effective amount of the cell of any one of embodiments 1-182.

Embodiment 184: The method of embodiment 183, wherein the cell is not originally derived from the patient.

Embodiment 185: The method of embodiment 183, wherein the cell is administered alone or in combination with one or more therapeutic agents.

Embodiment 186: The method of any one of embodiments 183-185, wherein the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer and wherein the autoimmune disease is selected from the group consisting of systemic lupus erythematosus, rheumatoid arthritis, myositis, myasthenia gravis, multiple sclerosis, Sjogren's syndrome, psoriasis, and inflammatory bowel disease.

Embodiment 187: An isolated immune cell engineered to have CD58 expression or activity that is at least 10% lower as compared to a corresponding non-engineered immune cell, that is also engineered to have RFX5 expression or activity that is at least 10% lower as compared to a corresponding non-engineered immune cell, and that is also engineered to have TGFBR2 expression or activity that is at least 10% lower as compared to a corresponding non-engineered immune cell.

Embodiment 188: The isolated cell of embodiment 189, further engineered to have ICAM1 expression or activity that is at least 10% lower as compared to a corresponding non-engineered immune cell.

Embodiment 189: A method for preparing an allogeneic immune cell with reduced activity in inducing graft-versus-host disease (GVHD) or host rejection, comprising reducing, in the cell, the expression or activity of CD58 by at least 10% as compared to a corresponding non-engineered immune cell, reducing, in the cell, the expression or activity of RFX5 by at least 10% as compared to a corresponding non-engineered cell, and reducing, in the cell, the expression or activity of TGFBR2 by at least 10% as compared to a corresponding non-engineered cell.

Embodiment 190: The method of embodiment 191, further comprising reducing, in the cell, the expression or activity of ICAM1 by at least 10% as compared to a corresponding non-engineered cell.

### Examples

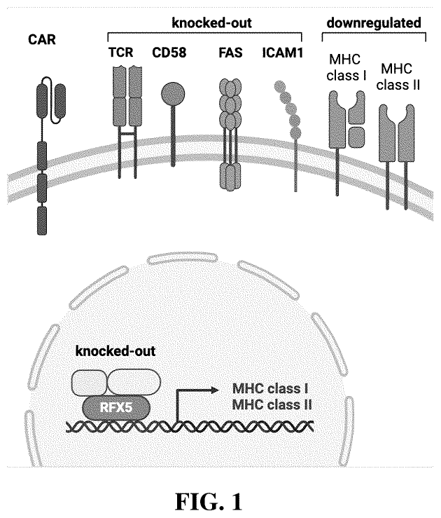

Example 1. Schematic depicting an allogeneic T cell engineered to evade immune rejection by deletion of CD58, FAS, ICAM1, RFX5, and a TCR gene such as TRAC or CD3E combined with expression chimeric antigen receptors with specificity for one or more antigens (FIG. 1).

Example 2. Combinations of gene knock-outs to reduce immune rejection of allogeneic T cells. The TCR gene can include any chain of the T cell receptor including TRAC, TRBC, CD3E, CD3D, CD3G, CD3Z.

TABLE 5
Combinations of gene knock-outs

| Combi- | | | | | |
|---|---|---|---|---|---|
| nation | TCR gene | CD58 | RFX5 | FAS | ICAM1 |
| 1 | TCR gene | CD58 | RFX5 | | |
| 2 | TCR gene | CD58 | RFX5 | FAS | |
| 3 | TCR gene | CD58 | RFX5 | | ICAM1 |
| 4 | TCR gene | CD58 | RFX5 | FAS | ICAM1 |
| 5 | TCR gene | CD58 | | | |
| 6 | TCR gene | CD58 | | FAS | |
| 7 | TCR gene | CD58 | | | ICAM1 |
| 8 | TCR gene | CD58 | | FAS | ICAM1 |

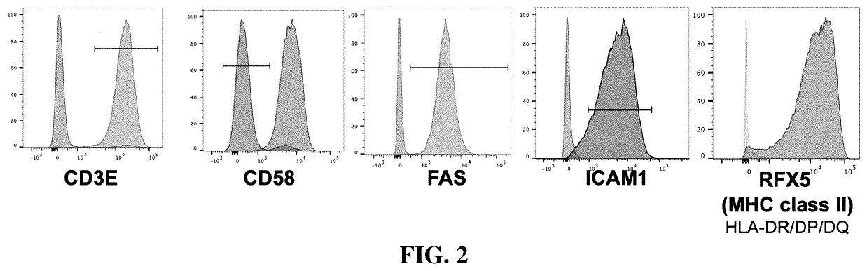

Example 3. Experimental data demonstrating highly efficient multiplex editing of 2, 3, 4, or 5 gene knockouts in human primary T cells using base editing. Loss of CD3E, CD58, FAS, and ICAM1 protein on the cell surface was assessed by flow cytometry. Loss of B2M was assessed by flow cytometry using antibodies specific for MHC class I. Loss of RFX5 was assessed by PCR and Sanger sequencing of the editing site. Panel A is a table showing editing efficiencies for each combination of gene edits. Panel B is representative histograms of flow cytometric analysis demonstrating loss of protein expression on the cell surface. The peak on the left is the gene edited sample, while the peak on the right is a non-edited wild type control sample. (FIG. 2).

### CD4 and CD8 T Cell Isolation

CD4 and CD8 T cells were isolated from healthy donor PBMC with CD4 and CD8 CliniMACS® beads and CliniMACS® instrument. Cells were frozen at 25×10⁶ cells/mL and frozen down in CryoStor® cell cryopreservation media (sourced from Sigma Aldrich®) and stored under liquid nitrogen.

### T Cell Culture and Activation

Healthy donor T cells were activated with plate-bound MACS GMP CD3 Pure (1.23 ug/mL for coating) and soluble mouse anti-human CD28 antibody (1 ug/mL final concentration) at 1×10⁶ cells/mL on day 0 in CTS™ OpTmizer™ media supplemented with CTS™ OpTmizer™ Cell SR, CTS™ OpTmizer™ T cell expansion supplement, Pen/Strep/Glutamine, and 300 IU/mL IL-2.

### CAR Transduction

One day following T cell activation, lentivirus encoding a CAR transgene was added to the culture medium with a multiplicity of infection of five.

### Base Editing

Seventy-Two hours after activation, T cells were electroporated using a Lonza 4D Nucleofector® X unit and a Lonza P3 Primary Cell and a P3 Primary Cell 4D-Nucleofector™ X Kit according to the manufacturer's instructions. mRNA encoding a adenine base editor was synthesized with N1-Methyl-Pseudo-U modification sourced from TriLink Biotechnologies, Inc. The adenine base editor was comprised of Cas9 nickase fused to deoxyadenosine deaminase derived from TadA. sgRNA that are compatible with Cas9 were obtained from Synthego with standard modifications consisting of 2′-O-methyl analogs (OMe) on the first and last three bases, and 3′ phosphorothioate internucleotide linkages (PS) between the first three and last two bases. 1×10⁶ T cells, 1 μg of mRNA, and 5 μg of sgRNA were combined and electroporated with pulse code EO115 in a 20 μl cuvette. sgRNA sequences are provided in Table 2. T cells were transferred to media as described above supplemented with 5% human AB serum sourced from Valley Biomedical, Inc.

### Flow Cytometry

Four days after electroporation, T cells were collected into 96 well plates and incubated with BD Pharmingen™ Human BD Fc Block and LIVE/DEAD™ Fixable Far Red Dead Cell Stain Kit sourced from Thermo Fisher Scientific, Inc. The protocol was in accordance to the manufacturer's instructions. Antibody staining was performed in BD Stain Buffer for 30 minutes at 4° C. Cells were washed in twice with BD stain buffer and resuspended in BD stain buffer prior to acquisition. Flow cytometry data was collected on BD FACSymphony™ A5 Cell Analyzer with BD FACS DIVA software and data was analyzed using FlowJo™ software sourced from BD Biosciences, Inc. Antibodies are listed: HLA-A/B/C (Clone G46-2.6, Biolegend #361714), HLA-DR/DP/DQ (Clone Tu39, BD #565332), ICAM1 (Clone HA58, Biolegend #353108), FAS (Clone DX2, Biolegend #305624), CD58 (Clone TS2/9 Biolegend #330928), and CD3E (Clone UCHT1, #330412).

Analysis of editing by Polymerase Chain Reaction (PCR) and Sanger Sequencing

Cell pellets were collected 4 days after electroporation and treated with QuickExtract™ DNA Extraction Solution (sourced from Biosearch Technologies, Inc.) according to the manufacturer's instructions. PCR was performed with Phusion® Hot Start Flex 2× Master Mix and DNA primers flanking the target region (sourced from Integrated DNA Technologies, Inc.). RFX5 was amplified with primers 5′ GTTTTTGAGGGGGAGGAGGG 3′ (SEQ ID NO: 51) and 5′ ACCACCGGGGAAATTCGGAG 3′ (SEQ ID NO: 52). Sanger sequencing of PCR products was analyzed using BEAT (https://hanlab.cc/beat/).

TABLE 6
Gene knockout efficiencies in CAR-T cells generated with
multiplexed base editing. CD3E, B2M, CD58, FAS, and ICAM1
were assessed by loss of TRCalpha/beta, HLA-ABC, CD58,
FAS, and ICAM1 protein using flow cytometry. RFX5 editing
efficiency was assessed by PCR and Sanger sequencing.
Representative results from one donor are shown.

| Multiplex gene | % editing efficiency | | | | | |
|---|---|---|---|---|---|---|
| knock outs | CD3E | B2M | CD58 | FAS | ICAM1 | RFX5 |
| CD3E AAVS1 | 95.4 | | | | | |
| CD3E B2M | 98.0 | 97.4 | | | | |
| CD3E CD58 RFX5 | 94.5 | | 95.0 | | | 97.0 |
| CD3E CD58 ICAM1 | 90.7 | | 94.3 | | 96.2 | |
| CD3E CD58 RFX5 | 92.5 | | 97.4 | | 99.0 | 97.0 |
| ICAM1 | | | | | | |
| CD3E CD58 FAS | 92.6 | | 97.4 | 96.9 | 98.7 | |
| ICAM1 | | | | | | |
| CD3E CD58 RFX5 | 95.3 | | 96.8 | 96.0 | 97.9 | 99.0 |
| FAS ICAM1 | | | | | | |

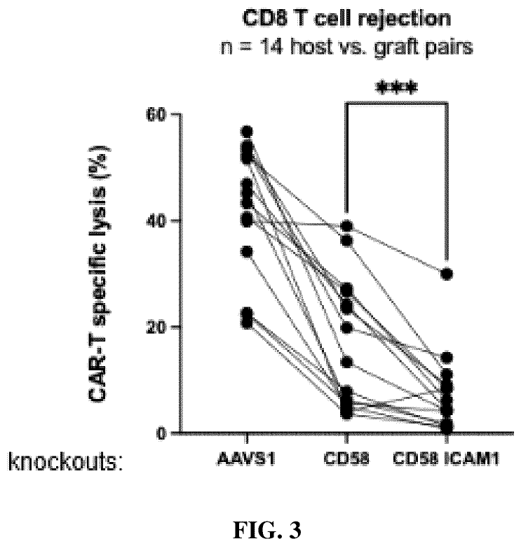

Example 4. Experimental data demonstrating deletion of ICAM1 in combination with CD58 protects from CD8 T cell rejection more than CD58 deletion alone. Allogeneic CAR-T cells were co-cultured with CD8 T cells from mismatched donors at 10:1 E:T ratio for 96 hours. Specific lysis was determined by flow cytometry. n=14 host vs. graft pairs from independent experiments. Wilcoxon matched-pairs signed rank test; *** indicates P<0.001. (FIG. 3)

### Preparation of Primed CD8 T Cells

CD4 and CD8 T cells were isolated from healthy donor PBMC with CD4 and CD8 CliniMACS® beads and CliniMACS® instrument. Cells were frozen at 25×10⁶ cells/mL and frozen down in CryoStor® cell cryopreservation media (sourced from Sigma Aldrich®) and stored under liquid nitrogen. Healthy donor T cells were activated with plate-bound MACS GMP CD3 Pure (1.23 ug/mL for coating) and soluble mouse anti-human CD28 antibody (1 ug/mL final concentration) at 1×10⁶ cells/mL on day 0 in CTS™ OpTmizer™ media supplemented with CTS™ OpTmizer™ Cell SR, CTS™ OpTmizer™ T cell expansion supplement, Pen/Strep/Glutamine, and 300 IU/mL IL-2. Cells were washed on day 3 and cultured until day 10. Primed CD8 T cells were isolated 10 days post-activation with CD8 CliniMACS® beads and CliniMACS® instrument. Primed CD8 T cells were frozen at 25×10⁶ cells/mL and frozen down in CryoStor® cell cryopreservation media (sourced from Sigma Aldrich®) and stored under liquid nitrogen.

### CD8 T Cell Rejection Assay

CAR T cells were generated as described in Example 3. CAR-T cells and primed T cells were thawed and rested overnight in RPMI supplemented with 10% Fetal Bovine Serum, Pen/Strep/Glutamine, and 100 IU/mL IL-2. Co-cultures were performed with 10,000 CAR-T cells labeled with Cell Trace Violet (Invitrogen) and 100,000 primed CD8 T cells for 96 hours in 96-well tissue culture plates. Cells were stained with Live/Dead Fixable Far Red (Invitrogen) and viability of CAR-T cells was determined by flow cytometry. Specific lysis was calculated as follows: Specific lysis=(% viable targets-only control)−(% viable with CD8 T cells).

TABLE 7
Specific lysis of gene edited CAR-T cells following
96-hour coculture with CD8 T cells from HLA
mismatched donors as described above.

| Host vs | AAVS1 | CD58 | CD58 |
|---|---|---|---|
| graft pair | KO | KO | ICAM1 KO |
| 1 | 22.57 | 7.87 | 1.3 |
| 2 | 20.77 | 3.63 | 1.23 |
| 3 | 56.8 | 13.33 | 4.27 |
| 4 | 54.06667 | 23.43333 | 9.1 |
| 5 | 43.33333 | 27.26667 | 6.2 |
| 6 | 53.33333 | 19.83333 | 14.23333 |
| 7 | 52.26667 | 36.23333 | 11.06667 |
| 8 | 34.2 | 5 | 0.8 |
| 9 | 45.2 | 6.2 | 1.9 |
| 10 | 51.7 | 4.1 | 8.4 |
| 11 | 22.5 | 5.6 | 4.2 |
| 12 | 40.6 | 26.6 | 8.4 |
| 13 | 39.9 | 39 | 30 |
| 14 | 46.9 | 24.2 | 4.5 |

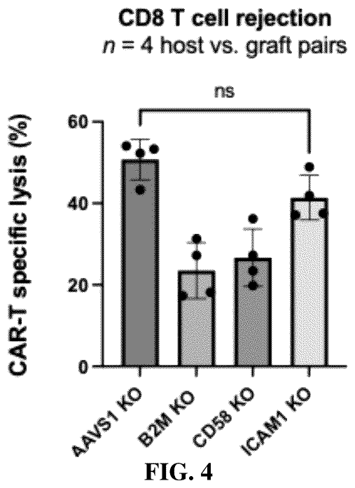

Example 5. Experimental data showing ICAM1 knockout alone does not protect from CD8 T cell rejection. CD19/20 CAR-T cells were co-cultured with CD8 T cells from mismatched donors at 10:1 E:T ratio for 96 hours. Specific lysis of CAR-T cells was determined by flow cytometry. n=6 host vs. graft pairs from independent experiments. Wilcoxon matched-pairs signed rank test; * indicates P<0.05. (FIG. 4)

TABLE 8
Specific lysis of gene edited CAR-T cells following
96-hour coculture with CD8 T cells from HLA mismatched
donors as described in Example 5.

| Host vs. | AAVS1 | B2M | CD58 | ICAM1 |
|---|---|---|---|---|
| graft pair | KO | KO | KO | KO |
| 1 | 54.06667 | 18.2 | 23.43333 | 41.93333 |
| 2 | 43.33333 | 31.3 | 27.26667 | 48.93333 |
| 3 | 53.33333 | 17.3 | 19.83333 | 37.53333 |
| 4 | 52.26667 | 27.2 | 36.23333 | 37.13333 |

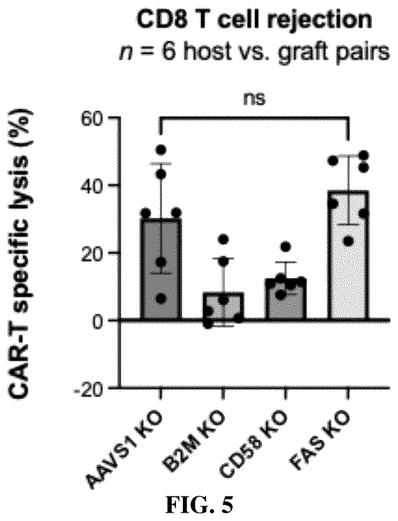

Example 6. Experimental data showing disruption of FAS alone does not protect from CD8 T cell rejection. Allogeneic CAR-T cells were co-cultured with CD8 T cells from mismatched donors at 10:1 E:T ratio for 96 hours. Specific lysis was determined by flow cytometry. n=6 host vs. graft pairs. Paired two-tailed T test; n.s. indicates P>0.05. (FIG. 5)

TABLE 9
Specific lysis of gene edited CAR-T cells following
96-hour coculture with CD8 T cells from HLA mismatched
donors as described in Example 6.

| Host vs | AAVS1 | B2M | CD58 | FAS |
|---|---|---|---|---|
| Graft pair | KO | KO | KO | KO |
| 1 | 31.90 | 2.77 | 10.50 | 23.50 |
| 2 | 31.77 | −0.97 | 10.97 | 31.60 |
| 3 | 43.33 | 24.00 | 12.47 | 34.57 |
| 4 | 6.53 | 0.73 | 7.57 | 47.30 |
| 5 | 17.33 | 6.07 | 11.53 | 45.30 |
| 6 | 50.47 | 17.53 | 21.80 | 48.80 |

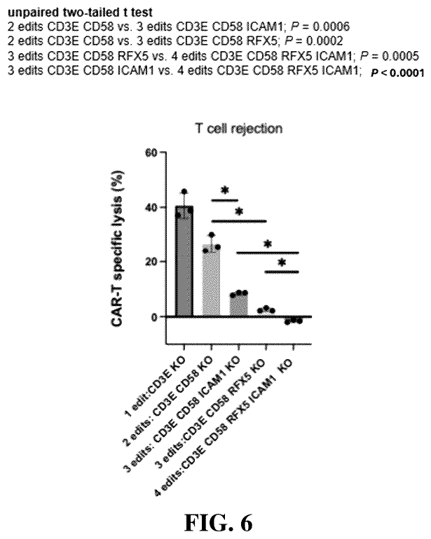

Example 7. Experimental data showing superior CD8 T cell evasion in CAR-T cells with 4 gene edits compared to 2 or 3 gene edits. CD19/20 CAR-T cells were co-cultured with CD8 T cells from mismatched donors at 10:1 E:T ratio for 96 hours. Specific lysis of CAR-T cells was determined by flow cytometry. n=3 replicate wells. Unpaired two-tailed T test; * indicates P<0.05. (FIG. 6).

TABLE 10
Specific lysis of gene edited CAR-T cells following
96-hour coculture with CD8 T cells from HLA mismatched
donors as described in Example 7.

| | Repli- | Repli- | Repli- |
|---|---|---|---|
| Condition | cate 1 | cate 2 | cate 3 |
| 1 edit: CD3E KO | 38.8 | 37.1 | 45.8 |
| B2M KO | 58 | 58.2 | 52.7 |
| 2 edits: CD3E CD58 KO | 25.6 | 30.1 | 24.1 |
| 3 edits: CD3E CD58 ICAM1 KO | 7.8 | 8.7 | 8.8 |
| 3 edits: CD3E CD58 RFX5 KO | 2.2 | 3.2 | 2.2 |
| 4 edits: CD3E CD58 RFX5 FAS KO | 12.7 | 11.7 | 13.3 |
| 4 edits: CD3E CD58 RFX5 ICAM1 KO | −1.6 | −1.2 | −2 |

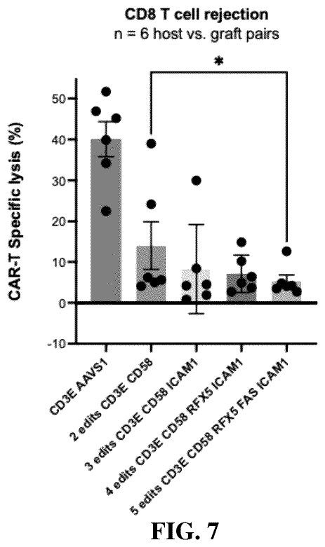

Example 8. Experimental data showing superior CD8 T cell evasion in CAR-T cells with 5 gene edits compared to 2 gene edits. CD19/20 CAR-T cells were co-cultured with CD8 T cells from mismatched donors at 10:1 E:T ratio for 96 hours. Specific lysis of CAR-T cells was determined by flow cytometry. n=6 host vs. graft pairs from independent experiments. Wilcoxon matched-pairs signed rank test; * indicates P<0.05. (FIG. 7)

TABLE 11
Specific lysis of gene edited CAR-T cells following 96-hour coculture
with CD8 T cells from HLA mismatched donors as described in Example 8.

| | Experi- | Experi- | Experi- | Experi- | Experi- | Experi- |
|---|---|---|---|---|---|---|
| Knockouts | ment 1 | ment 2 | ment 3 | ment 4 | ment 5 | ment 6 |
| CD3E AAVS1 KO | 34.2 | 45.2 | 51.7 | 22.5 | 39.9 | 46.9 |
| CD3E B2M | −1.4 | −3.9 | −2.3 | 1.8 | −5.4 | −2.2 |
| 2 edits CD3E CD58 KO | 5 | 6.2 | 4.1 | 5.6 | 39 | 24.2 |
| 3 edits CD3E CD58 ICAM1 | 0.8 | 1.9 | 8.4 | 4.2 | 30 | 4.5 |
| 4 edits CD3E CD58 RFX5 | 3.7 | 2.7 | 10.2 | 4.9 | 14.9 | 6.4 |
| ICAM1 | | | | | | |
| 5 edits CD3E CD58 RFX5 | 4.1 | 3.5 | 2.7 | 4.8 | 12.7 | 4.2 |
| FAS ICAM1 | | | | | | |

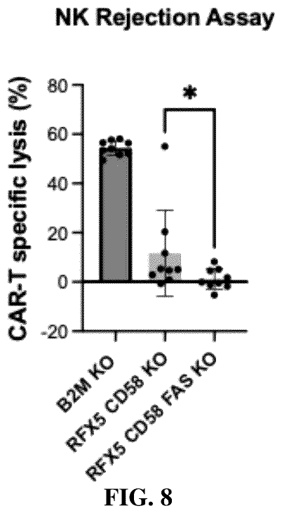

Example 9. Experimental data showing FAS knockout in combination with CD58 and RFX5 provides protection from NK cell rejection. Allogeneic CAR-T cells were co-cultured with NK cells from mismatched donors at 10:1 E:T ratio for 48 hours. Specific lysis was determined by flow cytometry. n=9 host vs. graft pairs. Wilcoxon matched-pairs signed rank test; * indicates P<0.05. (FIG. 8)

TABLE 12
Specific lysis of gene edited CAR-T cells following
48-hour coculture with NK cells from HLA mismatched
donors as described in Example 9.

| Host vs | B2M | RFX5 | RFX5 CD58 |
|---|---|---|---|
| Graft pair | KO | CD58 KO | FAS KO |
| 1 | 57.7 | 4.7 | −5.3 |
| 2 | 51.6 | 2.9 | −1.8 |
| 3 | 52.1 | −0.7 | 1.8 |
| 4 | 56.5 | 5.0 | −0.1 |
| 5 | 58.1 | 5.3 | −0.5 |
| 6 | 49.4 | 0.9 | 4.5 |
| 7 | 56.4 | 20.4 | 8.2 |
| 8 | 53.5 | 55.0 | −1.2 |
| 9 | 54.2 | 11.7 | 5.1 |

### Isolation of NK Cells

NK cells were isolated from healthy donor PBMC with NK CliniMACS® beads and CliniMACS® instrument. Cells were frozen at 25×10⁶ cells/mL and frozen down in CryoStor® cell cryopreservation media (sourced from Sigma Aldrich®) and stored under liquid nitrogen.

### NK Cell Rejection Assay

CAR T cells were generated as described in Example 3. CAR-T cells and NK cells were thawed and rested overnight in RPMI supplemented with 10% Fetal Bovine Serum, Pen/Strep/Glutamine, and 100 IU/mL IL-2. Co-cultures were performed with 10,000 CAR-T cells labeled with Cell Trace Violet (Invitrogen) and 100,000 NK cells for 48 hours in 96-well tissue culture plates with 100 IU/mL IL-2. Cells were stained with Live/Dead Fixable Far Red (Invitrogen) and viability of CAR-T cells was determined by flow cytometry. Specific lysis was calculated as follows: Specific lysis=(% viable targets-only control)−(% viable with NK cells).

Example 10. Experimental data demonstrating loss of CD58, ICAM, FAS, and RFX5 does not impact cytotoxic function of CAR-T cells. CD19/20 CAR-T cells were co-cultured with Raji tumor cells at various effector to target cell ratio. Tumor cells were stably transduced with luciferase. Target cell death was assessed by luciferase activity. NTD; non-transduced. No defects in cytotoxicity were observed compared to the AAVS1 edited control group.

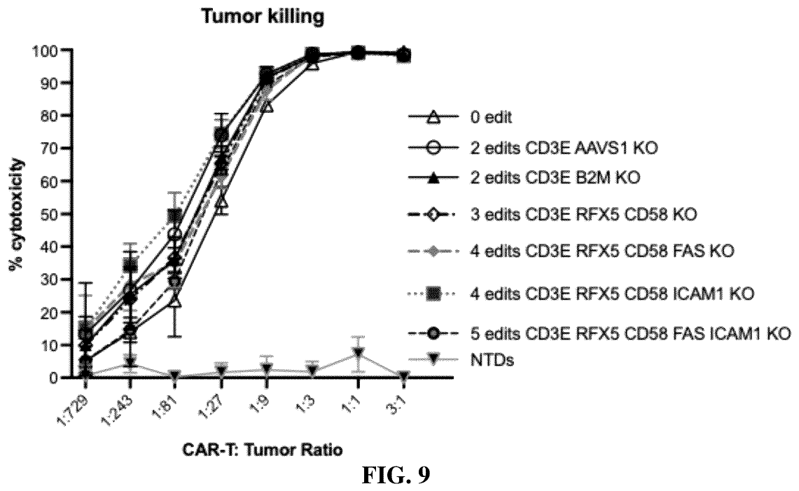

### (FIG. 9)

### Cytotoxicity Assay

CAR-T cells or non-transduced control cells were co-cultured in RPMI supplemented with 10% Fetal Bovine Serum, Pen/Strep/Glutamine in 96-well plates. 50,000 tumor cells were co-cultured with 150,000 or 50,000 or 16,600 CAR-T cells. Target cell death was assessed by luciferase activity. Briefly, the D-luciferin substrate was added to the co-culture wells at a final concentration of 0.14 mg/mL and plates were incubated at 37° C. in the dark for 10 minutes. Luminescent signal was read immediately after in a VarioSkan™ LUX or VarioSkan® Flash multimode microplate reader. T cell-mediated cytotoxicity was calculated as follows: % Cytotoxicity=[1-luciferase signal of (sample of interest/target alone control)]*100.

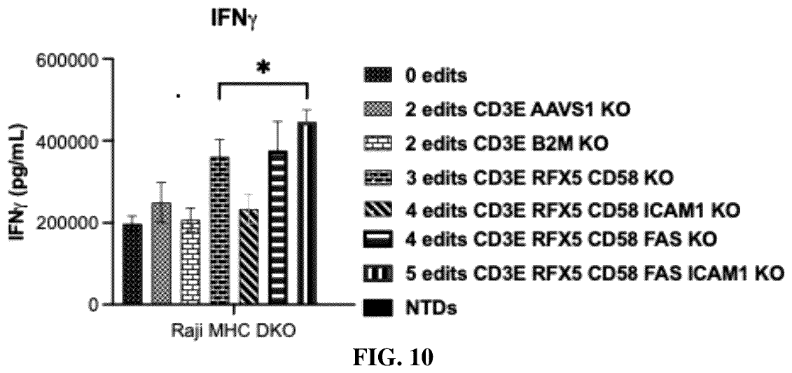

Example 11. Experimental data showing loss of CD58, ICAM, FAS, and RFX5 does not impair the cytokine production function of CAR-T cells. CD19/20 CAR-T cells were co-cultured with Raji tumor cells for 24 hours. All conditions were transduced with CAR except to the non-transduced (NTD) condition. Interferon gamma was quantified by MSD. n=3 replicate wells. unpaired T test; * indicates P<0.05. (FIG. 10)

TABLE 13
Release of Interferon gamma from gene edited CAR-T cells following
24 hour stimulation with tumor cells as described in example 11.

| | Repli- | Repli- | Repli- |
|---|---|---|---|
| Condition | cate 1 | cate 2 | cate 3 |
| 0 edits | 185481.6 | 188530.8559 | 220018.1 |
| 2 edits CD3E AAVS1 KO | 213984.5 | 233106.9214 | 175723.3 |
| 3 edits CD3E RFX5 CD58 KO | 342374.1 | 331769.8044 | 409625.4 |
| 4 edits CD3E RFX5 CD58 ICAM1 KO | 193066.9 | 262302.0037 | 247558.5 |
| 4 edits CD3E RFX5 CD58 FAS KO | 296059.2 | 427136.622 | 406431.8 |
| 5 edits CD3E RFX5 CD58 FAS ICAM1 KO | 478628.5 | 441200.2326 | 418534 |
| Non transduced | 0 | 1.566014359 | 0 |

### Cytokine Release Assay

CD19/20 CAR-T cells were co-cultured with Raji tumor cells at 1:1 effector to target cell ratio for 24 hours. 50,000 tumor cells and 50,000 CAR-T cells were co-cultured in RPMI supplemented with 10% Fetal Bovine Serum, Pen/Strep/Glutamine in 96-well plates. Cytokine abundance in cell culture supernatants was quantified by Meso Scale Discovery Electrochemiluminescence (MSD).

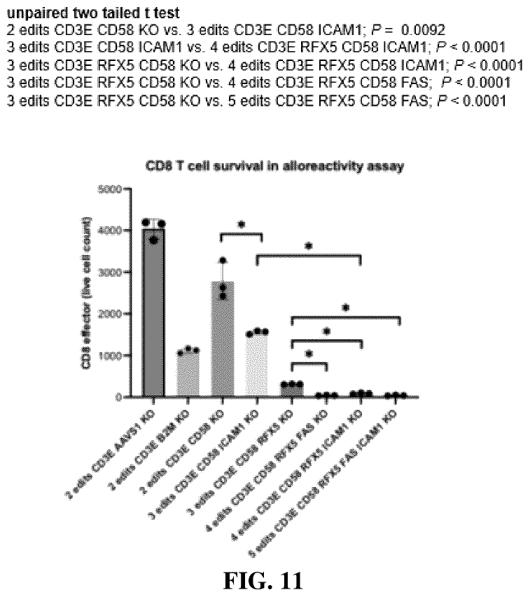

Example 12. Experimental data demonstrating the combination of CD58 and RFX5 knockout with ICAM1 and/or FAS knockout in CAR-T cells diminishes mismatched effector T cell survival. CAR-T cells with 4 or 5 edits were superior to those with 2 or 3 edits at evading recognition by CD8 T cells. Survival of CD8 T cells in a 96-hour co-culture with mismatched CAR-T cells was used as an indicator of a CD8 T cell immune response. The number of viable CD8 T cells was assessed by flow cytometry. n=3 replicate wells. Unpaired T test; * indicates P<0.05. (FIG. 11).

TABLE 14
Live CD8 effector cell count following 96-
hour co-culture with gene edited CAR-T cells.

| | Repli- | Repli- | Repli- |
|---|---|---|---|
| Condition | cate 1 | cate 2 | cate 3 |
| 2 edits CD3E AAVS1 KO | 4162 | 3767 | 4196 |
| 2 edits CD3E B2M KO | 1052 | 1164 | 1122 |
| 2 edits CD3E CD58 KO | 2430 | 2633 | 3286 |
| 3 edits CD3E CD58 ICAM1 KO | 1592 | 1511 | 1571 |
| 3 edits CD3E CD58 RFX5 KO | 304 | 317 | 311 |
| 4 edits CD3E CD58 RFX5 FAS KO | 49 | 35 | 42 |
| 4 edits CD3E CD58 RFX5 ICAM1 KO | 74 | 86 | 106 |
| 5 edits CD3E CD58 RFX5 FAS ICAM1 KO | 53 | 33 | 39 |

### Memory CD8 T Cell Persistence and Proliferation Assay

This assay is used to evaluate CD8 T cell recognition of allogeneic CAR-T cells. CAR T cells were generated as described in Example 3. Memory CD8 T cells were primed with CD3 and CD28 stimulation as described in Example 4. CAR-T cells and primed CD8 T cells were thawed and rested overnight in RPMI supplemented with 10% Fetal Bovine Serum, Pen/Strep/Glutamine without IL-2. Co-cultures were performed with 50,000 CAR-T cells labeled with Cell Trace Violet (Invitrogen) and 50,000 primed CD8 T cells labeled with carboxyfluorescein (CFSE, ThermoFisher Scientific) for 96 hours in 96-well tissue culture plates with 100 IU IL-2/mL. Cells were stained with Live/Dead Fixable Far Red (Invitrogen) and the absolute number of live CD8 T cells was determined by flow cytometry using the iQue® flow cytometer (Sartorius). Proliferation of CD8 T cells was determined by reduced CFSE staining, since cellular division dilutes the intensity of CFSE in the cell membrane.

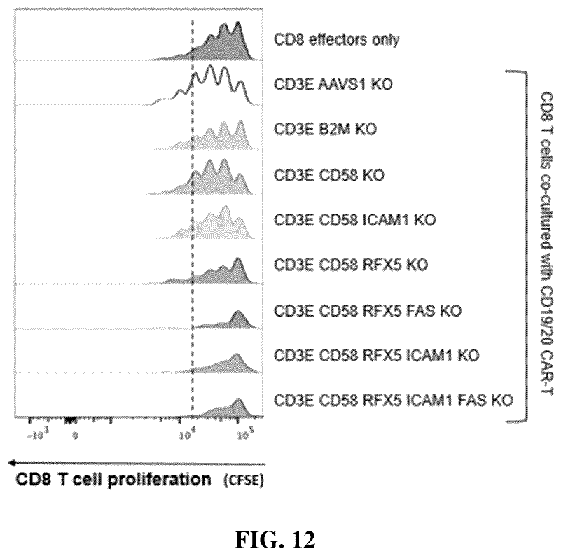

Example 13. Experimental data showing diminished CD8 T cell responses following exposure to CAR-T cells with 4 or 5 gene edits compared to 2 or 3 gene edits. The combination of CD58 and RFX5 knockout with ICAM1 and/or FAS knockout in CAR-T cells reduced mismatched effector T cell proliferation. Proliferation of CD8 T cells in response to CAR-T exposure is an indication of immune rejection. Proliferation of CD8 T cells was assessed by loss of Carboxyfluorescein succinimidyl ester (CFSE) staining using flow cytometry 96 hours after exposure to CAR-T cells. The assay was performed as described in Example 12. (FIG. 12)

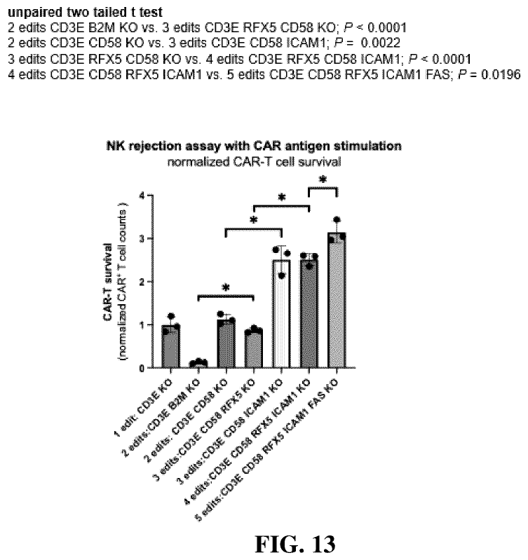

Example 14. Experimental data showing superior NK cell evasion in CAR-T cells with 4 or 5 gene edits compared to 2 or 3 gene edits. CD19/20 CAR-T cells were challenged three times with exposure to allogeneic NK cells and CD19⁺ CD20⁺ Raji cells at 4-day intervals. 10,000 CAR-T cells were plated with 3,000 Raji cells and 50,000 primary NK cells in RPMI supplemented with 100 U IL-2/mL. CAR-T cell survival was assessed on the final day by flow cytometry. Absolute live CAR-T cell counts were normalized to the 1 edit CD3E KO control. n=3 replicate wells. Unpaired two-tailed T test; * indicates P<0.05. (FIG. 13)

TABLE 15
Normalized CAR-T cell survival following serial
exposure to allogeneic NK cells in the presence
of tumor cells as described in Example 14.

| | repli- | repli- | repli- |
|---|---|---|---|
| Condition | cate 1 | cate 2 | cate 3 |
| 1 edit: CD3E KO | 1.201 | 0.959 | 0.843 |
| 2 edits: CD3E B2M KO | 0.155 | 0.107 | 0.126 |
| 3 edits: CD3E CD58 RFX5 KO | 0.930 | 0.862 | 0.823 |
| 4 edits: CD3E CD58 RFX5 ICAM1 KO | 2.605 | 2.586 | 2.354 |
| 5 edits: CD3E CD58 RFX5 ICAM1 FAS KO | 3.438 | 3.041 | 2.964 |
| 2 edits: CD3E CD58 KO | 1.027 | 1.104 | 1.249 |
| 3 edits: CD3E CD58 ICAM1 KO | 2.741 | 2.654 | 2.140 |

NK Rejection Assay with Serial Rechallenges

Gene edited CAR-T cells were generated as described in Example 3 and NK cells were isolated as described in Example 9. NK cells were labeled with Cell Trace Violet (Invitrogen) and CAR-T cells were labeled with carboxyfluorescein (CFSE, ThermoFisher Scientific). Serial co-culture was performed in RPMI media supplemented with Pen/Strep/Glutamine and 100 IU IL-2/mL with ten thousand CAR-T cells, three thousand Raji tumor cells, and fifty thousand NK cells in 96-well plates. At 24-hour intervals, an additional three thousand Raji tumor cells and fifty thousand NK cells were added to the wells. After 96 hours, the co-culture was stained with viability dye and the absolute number of viable of CAR-T cells was assessed by flow cytometry. Absolute CAR-T cell counts were then normalized to the CD3E KO control condition for visualization.

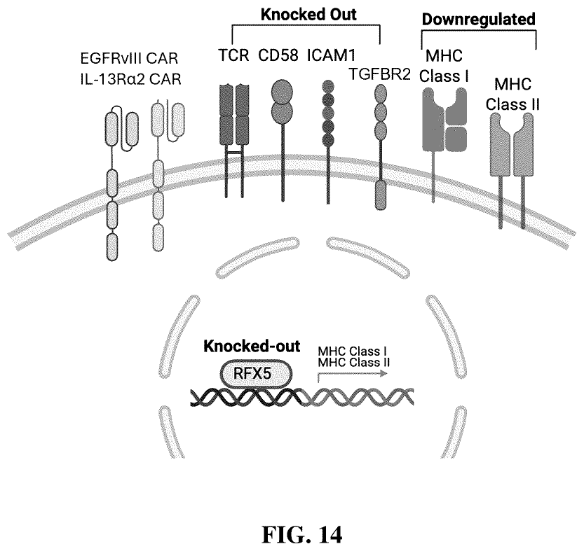

Example 15. FIG. 14 is a schematic depicting an allogeneic T cell engineered to evade immune rejection by deletion of CD58, ICAM1, and RFX5, to have resistance to TGFβ-mediated immunosuppression by deletion of TGFBR2, and to prevent graft versus host disease by deletion of a TCR gene such as TRAC or CD3E combined with expression chimeric antigen receptors with specificity for one or more antigens. T cell effector function and anti-tumor activity is suppressed by TGFβ in the tumor microenvironment. Deletion of the receptor TGFBR2 renders CAR-T cells insensitive to TGFβ signaling and increases therapeutic activity against tumors that produce TGFβ, such as glioblastoma.

Example 16. Table 16 lists combinations of gene knockout to reduce immune rejection of allogeneic T cells, prevent graft versus host disease, and protect from TGFB mediated immunosuppression. The TCR gene can include any chain of the T cell receptor including TRAC, TRBC, CD3E, CD3D, CD3G, CD3Z.

TABLE 16
Combinations of gene knock-outs.

| Combi- | | | | | |
|---|---|---|---|---|---|
| nation | TCR gene | CD58 | RFX5 | TGFBR2 | ICAM1 |
| 1 | TCR gene | CD58 | RFX5 | TGFBR2 | |
| 2 | TCR gene | CD58 | RFX5 | TGFBR2 | ICAM1 |

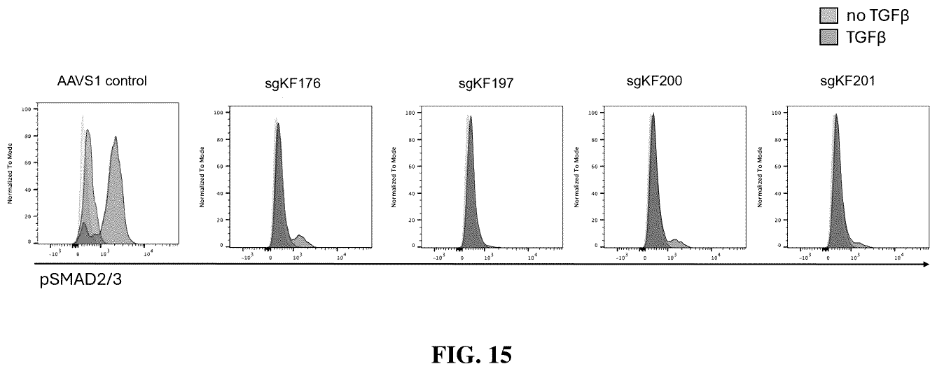

Example 17. This example discloses experimental data showing TGFBR2 knockout efficiency for various sgRNA sequences. Table 17 lists various sgRNA sequences used for the knockout of TGFBR2. Gene knockout resulted in functional loss of TGFβ downstream signaling when T cells were exposed to TGFβ-1. TGFβ signaling was assessed by intracellular staining with an antibody that recognizes phosphorylated serine residues on Smad2 and Smad3 residues phosphorylated (Smad2 pS465/pS467 and Smad3 pS423/pS425) followed by quantification by flow cytometry (Table 18, FIG. 15).

TABLE 17
Spacer sequences of sgRNAs designed to
target TGFBR2 with adenine base editors.

| SEQ | | | | |
|---|---|---|---|---|
| ID | | | | Design |
| NO: | Name | Spacer sequence | PAM | method |
| 53 | sgKF176 | CCATGGGTCGGGGGCTGCTC | NGG | manual |
| 54 | sgKF177 | CGGGGTCTGCCATGGGTCGG | NGG | manual |
| 55 | sgKF178 | UUACCUGCCCACUGUUAGCC | NGG | SpliceR |
| 56 | sgKF179 | TCACCCGACTTCTGAACGTG | NGG | SpliceR |
| 57 | sgKF194 | ACTCACCCGACTTCTGAACG | NG | SpliceR |
| 58 | sgKF195 | TGCTTACCATACAGCCACAC | NG | SpliceR |
| 59 | sgKF196 | CTCACCTTCTGAGAAGATGA | NG | SpliceR |
| 60 | sgKF197 | CCACTCACCCGACTTCTGAA | NG | SpliceR |
| 61 | sgKF198 | AAACTCACCTTCTGAGAAGA | NG | SpliceR |
| 62 | sgKF199 | CTCCTCAGTTAATAACGACA | NG | SpliceR |
| 63 | sgKF200 | CACTCTAGGAGAAAGAATGA | NG | SpliceR |
| 64 | sgKF201 | CTCTAGGAGAAAGAATGACG | NG | SpliceR |
| 65 | sgKF202 | CCCCATCAGAATATAACACC | NG | SpliceR |

TABLE 18
Gene knockout efficiencies in primary T cells assessed by
loss of phosphorylation of SMAD2 and SMAD3 TGFβ-1 exposure.

phospho SMAD2/3negative

| Gene KO | sgRNA | replicate 1 | replicate 2 |
|---|---|---|---|
| AAVS1 | | 6.9 | 8.2 |
| control | | | |
| TGFBR2 | sgKF176 | 86 | 87.6 |
| TGFBR2 | sgKF177 | 11.8 | 11.8 |
| TGFBR2 | sgKF178 | 7.1 | 7 |
| TGFBR2 | sgKF179 | 27.7 | 29 |
| TGFBR2 | sgKF194 | 61.3 | 60.3 |
| TGFBR2 | sgKF195 | 46.8 | 46.9 |
| TGFBR2 | sgKF196 | 10.7 | 10.1 |
| TGFBR2 | sgKF197 | 96.49 | 96.34 |
| TGFBR2 | sgKF198 | 9.4 | 10.4 |
| TGFBR2 | sgKF199 | 7.1 | 6.5 |
| TGFBR2 | sgKF200 | 89.6 | 89.6 |
| TGFBR2 | sgKF201 | 92.11 | 91.99 |
| TGFBR2 | sgKF202 | 13.5 | 14.3 |

### TGFβ Signaling Assay

Human primary T cells were base edited as described in Example 3. Four days after electroporation, T cells were collected into 96 well plates and incubated without serum for two hours. TGFβ-1 was supplemented to the culture medium at a concentration of 5 ng/mL for 30 minutes. Live/dead staining and Fc receptor blocking was performed as described in Example 3. Cells were fixed and permeabilized with BD Phosflow™ Perm Buffer I and III according to manufacturer's instructions. Intracellular staining of Smad2 pS465/pS467 and Smad3 pS423/pS425 was performed according to manufacturer's instructions. Flow cytometry data was collected on BD FACSymphony™ A5 Cell Analyzer with BD FACS DIVA software and data was analyzed using FlowJo™ software sourced from BD Biosciences, Inc.

Example 18. Table 19 shows experimental data demonstrating efficient combination of gene knockouts in human CAR-T cells expressing chimeric antigen receptors targeting a mutated form of EGFR (EGFRvIII) and IL-13Ra2. These data demonstrate manufacturing of multiplexed gene edited CAR-T cells with gene deletions to prevent immune rejection and disrupt TGFβ signaling combined with CARs targeting antigens that can be used for the treatment of glioblastoma.

TABLE 19
Gene knockout efficiencies in CAR-T cells generated with multiplexed
base editing. CD3E, CD58, ICAM1 were assessed by loss of CD3E,
CD58, ICAM1 protein using flow cytometry gating. The percentage
of cells that do not express the protein on the cell surface
is indicated in the table. RFX5 and TGFBR2 editing efficiency
was assessed by PCR and Sanger sequencing. Representative
results from one donor are shown.

| Multiplex | | | | | |
|---|---|---|---|---|---|
| gene knock | % editing efficiency | | | | |
| outs | CD3E | CD58 | RFX5 | ICAM1 | TGFBR2 |
| CD3E CD58 | 94.5 | 95 | 91 | 97.89 | 0 |
| RFX5 ICAM1 | | | | | |
| CD3E CD58 | 93.4 | 88 | 91 | 98.06 | 60 |
| RFX5 ICAM1 | | | | | |
| TGFBR1 | | | | | |

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. An isolated immune cell engineered to have CD58 expression or activity that is at least 10% lower as compared to a corresponding non-engineered immune cell and engineered to have FAS expression or activity that is at least 10% lower as compared to a corresponding non-engineered immune cell.

2. The cell of claim 1, further engineered to have ICAM1 expression or activity that is at least 10% lower as compared to a corresponding non-engineered immune cell.

3. The cell of claim 1, further engineered to have RFX5 expression or activity that is at least 10% lower as compared to a corresponding non-engineered immune cell.

4. The cell of claim 1, further engineered to have TGFBR2 expression or activity that is at least 10% lower as compared to a corresponding non-engineered immune cell.

5.-46. (canceled)

47. An isolated immune cell engineered to have CD58 expression or activity that is at least 10% lower as compared to a corresponding non-engineered immune cell and engineered to have ICAM1 expression or activity that is at least 10% lower as compared to a corresponding non-engineered immune cell.

48. The cell of claim 47, further engineered to have RFX5 expression or activity that is at least 10% lower as compared to a corresponding non-engineered immune cell.

49. The cell of claim 47, further engineered to have TGFBR2 expression or activity that is at least 10% lower as compared to a corresponding non-engineered immune cell.

50.-83. (canceled)

84. The cell of claim 47, wherein the cell is a T cell or a natural killer (NK) cell, and wherein the cell is a human cell.

85. (canceled)

86. The cell of claim 47, wherein the cell comprises an exogenous polynucleotide encoding a chimeric antigen receptor (CAR) or a T-cell receptor (TCR); wherein the TCR gene is one of TRAC, TRBC, CD3E, CD3G, CD3D, or CD3Z, and wherein the CAR recognizes at least one of CD19, CD20, CD22, BCMA, TACI, EGFRVIII, IL13RA, GPC3, GPC2, and CD38.

87. (canceled)

88. (canceled)

89. The cell of claim 47, wherein the expression or activity of endogenous CD3E is also reduced in the cell.

90. The cell of claim 47, wherein the endogenous B2M (Beta-2-microglobulin) gene is not engineered, or wherein the cell has normal activity of B2M.

91. The cell of claim 47, wherein the cell has normal activity of MHC Class I.

92. The cell of claim 47, wherein the reduction in CD58 expression or activity is achieved by:
(a) editing of the endogenous gene encoding CD58,
(b) expression of an inhibitory RNA,
or (c) an inhibitor, preferably an antibody.

93. (canceled)

94. The cell of claim 92, wherein the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editing (ABE8 or BE4MAX).

95. (canceled)

96. (canceled)

97. The cell of claim 47, wherein, upon administration to a patient, the cell is characterized by reduced activity in inducing graft-versus-host disease (GVHD) or host rejection.

98. The cell of claim 47, wherein, upon administration to a patient, the cell is characterized by reduced killing by MHC-mismatched CD8⁺ T cells and/or NK cells.

99. A method for preparing an allogeneic immune cell with reduced activity in inducing graft-versus-host disease (GVHD) or host rejection, comprising reducing, in the cell, the expression or activity of CD58 by at least 10% as compared to a corresponding non-engineered immune cell, further comprising reducing, in the cell, the expression or activity of FAS by at least 10% as compared to a corresponding non-engineered cell.

100.-115. (canceled)

116. The method of claim 99, comprising reducing, in the cell, the expression or activity of ICAM1 by at least 10% as compared to a corresponding non-engineered immune cell.

117.-124. (canceled)

125. The method of claim 99, comprising reducing, in the cell, the expression or activity of RFX5 by at least 10% as compared to a corresponding non-engineered immune cell.

126.-133. (canceled)

134. A method for preparing an allogeneic immune cell with reduced activity in inducing graft-versus-host disease (GVHD) or host rejection, comprising reducing, in the cell, the expression or activity of CD58 by at least 10% as compared to a corresponding non-engineered immune cell, further comprising reducing, in the cell, the expression or activity of ICAM1 by at least 10% as compared to a corresponding non-engineered cell.

135. The method of claim 134, wherein the cell is further engineered to have RFX5 expression or activity that is at least 10% lower as compared to a corresponding non-engineered immune cell.

136. The method of claim 134, wherein the cell is further engineered to have TGFBR2 expression or activity that is at least 10% lower as compared to a corresponding non-engineered immune cell.

137.-167. (canceled)

168. The method of claim 134, wherein the cell is a T cell or a natural killer (NK) cell, and wherein the cell is a human cell.

169. (canceled)

170. The method of claim 134, wherein the method further comprises introducing into the cell an exogenous polynucleotide encoding a chimeric antigen receptor (CAR) or a T-cell receptor (TCR); wherein the TCR gene is one of TRAC, TRBC, CD3E, CD3G, CD3D, or CD3Z, and wherein the CAR recognizes at least one of CD19, CD20, CD22, BCMA, TACI, EGFRVIII, IL13RA, GPC3, GPC2, and CD38.

171. (canceled)

172. (canceled)

173. The method of claim 134, wherein the method further comprises reducing the expression or activity of CD3E in the cell.

174. The method of claim 134, wherein the endogenous B2M (Beta-2-microglobulin) gene in the cell is not engineered, or wherein the cell has normal activity of B2M.

175. The method of claim 134, wherein the cell has normal activity of MHC Class I.

176. The method of claim 134, wherein the reduction in CD58 expression or activity is achieved by:
(a) editing of the endogenous gene encoding CD58,
(b) expression of an inhibitory RNA, or
(c) an inhibitor, preferably an antibody.

177. (canceled)

178. The method of claim 176, wherein the editing is by CRISPR/Cas9, a zinc finger nuclease (ZFN), a transcription activator-like effector nuclease (TALEN), a MegaTAL, a meganuclease, Cpf1, homologous recombination, a single stranded oligodeoxynucleotide (ssODN), or base editing (ABE8 or BE4MAX).

179.-182. (canceled)

183. A method for treating cancer and/or autoimmune diseases in a patient in need thereof, comprising administering to the patient a therapeutically effective amount of the cell of claim 1.

184. The method of claim 183, wherein the cell is not originally derived from the patient.

185.-190. (canceled)


SEQUENCE LISTING (66 sequences)

SEQ ID NO: 1 - RNA, 20 nt, synthetic construct
  source 1..20: mol_type = other RNA
actcacgctggatagcctcc

SEQ ID NO: 2 - RNA, 113 nt, synthetic construct
  source 1..113: mol_type = other RNA
actcacgctggatagcctccgtttaagagctaagctggaaacagcatagcaagtttaaataaggctagtccgttatcaacttgaaaaagtggcaccgagtcggtgcttttttt

SEQ ID NO: 3 - RNA, 20 nt, synthetic construct
  source 1..20: mol_type = other RNA
ctcaccgctgcttgggatac

SEQ ID NO: 4 - RNA, 113 nt, synthetic construct
  source 1..113: mol_type = other RNA
ctcaccgctgcttgggatacgtttaagagctaagctggaaacagcatagcaagtttaaataaggctagtccgttatcaacttgaaaaagtggcaccgagtcggtgcttttttt

SEQ ID NO: 5 - RNA, 20 nt, synthetic construct
  source 1..20: mol_type = other RNA
gtacttacgaaatggtacct

SEQ ID NO: 6 - RNA, 113 nt, synthetic construct
  source 1..113: mol_type = other RNA
gtacttacgaaatggtacctgtttaagagctaagctggaaacagcatagcaagtttaaataaggctagtccgttatcaacttgaaaaagtggcaccgagtcggtgcttttttt

SEQ ID NO: 7 - RNA, 20 nt, synthetic construct
  source 1..20: mol_type = other RNA
ctggattacctcttgccctc

SEQ ID NO: 8 - RNA, 113 nt, synthetic construct
  source 1..113: mol_type = other RNA
ctggattacctcttgccctcgtttaagagctaagctggaaacagcatagcaagtttaaataaggctagtccgttatcaacttgaaaaagtggcaccgagtcggtgcttttttt

SEQ ID NO: 9 - RNA, 20 nt, synthetic construct
  source 1..20: mol_type = other RNA
aacttaccccaaacaattag

SEQ ID NO: 10 - RNA, 113 nt, synthetic construct
  source 1..113: mol_type = other RNA
aacttaccccaaacaattaggtttaagagctaagctggaaacagcatagcaagtttaaataaggctagtccgttatcaacttgaaaaagtggcaccgagtcggtgcttttttt

SEQ ID NO: 11 - RNA, 20 nt, synthetic construct
  source 1..20: mol_type = other RNA
tcctgcagggactccagaac

SEQ ID NO: 12 - RNA, 113 nt, synthetic construct
  source 1..113: mol_type = other RNA
tcctgcagggactccagaacgtttaagagctaagctggaaacagcatagcaagtttaaataaggctagtccgttatcaacttgaaaaagtggcaccgagtcggtgcttttttt

SEQ ID NO: 13 - RNA, 20 nt, synthetic construct
  source 1..20: mol_type = other RNA
gttaatgtggctctggttct

SEQ ID NO: 14 - RNA, 113 nt, synthetic construct
  source 1..113: mol_type = other RNA
gttaatgtggctctggttctgtttaagagctaagctggaaacagcatagcaagtttaaataaggctagtccgttatcaacttgaaaaagtggcaccgagtcggtgcttttttt

SEQ ID NO: 15 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 16 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 17 - protein, 250 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 18 - protein, 616 aa, Homo sapiens
MAEDEPDAKSPKTGGRAPPGGAEAGEPTTLLQRLRGTISKAVQNKVEGILQDVQKFSDNDKLYLYLQLPSGPTTGDKSSEPSTLSNEEYMYAYRWIRNHLEEHTDTCLPKQSVYDAYRKYCESLACCRPLSTANFGKIIREIFPDIKARRLGGRGQSKYCYSGIRRKTLVSMPPLPGLDLKGSESPEMGPEVTPAPRDELVEAACALTCDWAERILKRSFSSIVEVARFLLQQHLISARSAHAHVLKAMGLAEEDEHAPRERSSKPKNGLENPEGGAHKKPERLAQPPKDLEARTGAGPLARGERKKSVVESSAPGANNLQVNALVARLPLLLPRAPRSLIPPIPVSPPILAPRLSSGALKVATLPLSSRAGAPPAAVPIINMILPTVPALPGPGPGPGRAPPGGLTQPRGTENREVGIGGDQGPHDKGVKRTAEVPVSEASGQAPPAKAAKQDIEDTASDAKRKRGRPRKKSGGSGERNSTPLKSAAAMESAQSSRLPWETWGSGGEGNSAGGAERPGPMGEAEKGAVLAQGQGDGTVSKGGRGPGSQHTKEAEDKIPLVPSKVSVIKGSRSQKEAFPLAKGEVDTAPQGNKDLKEHVLQSSLSQEHKDPKATPP

SEQ ID NO: 19 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 20 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 21 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 22 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 24 - protein, 120 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARGGGSWYSNWFDPWGQGTMVTVSS

SEQ ID NO: 25 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQDRSLPPTFGGGTKVEIK

SEQ ID NO: 26 - protein, 121 aa, synthetic construct
QVQLQQWGAGLLKPSETLSLTCAVYGGSFSGIHWNWIRQPPGKGLEWIGDIDTSGSTNYNPSLKSRVTISVDTSKNQFSLKLSSVTAADTAVYYCARLGQESATYLGMDVWGQGTTVTVSS

SEQ ID NO: 27 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYTYPFTFGGGTKVEIK

SEQ ID NO: 28 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSS

SEQ ID NO: 29 - protein, 107 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIK

SEQ ID NO: 30 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 31 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 32 - protein, 124 aa, synthetic construct
QVQLQESGPGLVKPSETLSLTCTVSGGSISSPDHYWGWIRQPPGKGLEWIGSIYASGSTFYNPSLKSRVTISVDTSKNQFSLKLSSVTAADTAVYYCARETDYSSGMGYGMDVWGQGTTVTVSS

SEQ ID NO: 33 - protein, 107 aa, synthetic construct; identical to SEQ ID NO: 29

SEQ ID NO: 34 - protein, 123 aa, synthetic construct
QITLKESGPTLVKPTQTLTLTCTFSGFSLDTEGVGVGWIRQPPGKALEWLALIYFNDQKRYSPSLKSRLTITKDTSKNQVVLTMTNMDPVDTAVYYCARDTGYSRWYYGMDVWGQGTTVTVSS

SEQ ID NO: 35 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAYAYPITFGGGTKVEIK

SEQ ID NO: 36 - protein, 127 aa, synthetic construct
QVQLQQWGAGLLKPSETLSLTCAVYGGSFEKYYWSWIRQPPGKGLEWIGEIYHSGLTNYNPSLKSRVTISVDTSKNQFSLKLSSVTAADTAVYYCARVRYDSSDSYYYSYDYGMDVWGQGTTVTVSS

SEQ ID NO: 37 - protein, 113 aa, synthetic construct
DIVLTQSPDSLAVSLGERATINCKSSQSVLYSSNNKNYLAWYQQKPGQPPKLLIYWASSRESGVPDRFSGSGSGTDFTLTISSLQAEDVAVYYCQQSYSFPWTFGGGTKVEIK

SEQ ID NO: 38 - protein, 127 aa, synthetic construct
QVQLQQWGAGLLKPSETLSLTCAVYGGSFSRYVWSWIRQPPGKGLEWIGEIDSSGKTNYNPSLKSRVTISVDTSKNQFSLKLSSVTAADTAVYYCARVRYDSSDSYYYSYDYGMDVWGQGTTVTVSS

SEQ ID NO: 39 - protein, 113 aa, synthetic construct; identical to SEQ ID NO: 37

SEQ ID NO: 40 - protein, 127 aa, synthetic construct
QVQLQQWGAGLLKPSETLSLTCAVYGGSFSGYAWSWIRQPPGKGLEWIGEIDHRGFTNYNPSLKSRVTISVDTSKNQFSLKLSSVTAADTAVYYCARVRYDSSDSYYYSYDYGMDVWGQGTTVTVSS

SEQ ID NO: 41 - protein, 113 aa, synthetic construct; identical to SEQ ID NO: 37

SEQ ID NO: 42 - protein, 123 aa, synthetic construct
QVQLQQWGAGLLKPSETLSLTCAVYGGSFQKYYWSWIRQPPGKGLEWIGEIDTSGFTNYNPSLKSRVTISVDTSKNQFSLKLSSVTAADTAVYYCARVGRYSYGYYITAFDIWGQGTTVTVSS

SEQ ID NO: 43 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQHYSFPFTFGGGTKVEIK

SEQ ID NO: 44 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 45 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 46 - protein, 120 aa, synthetic construct
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 47 - protein, 107 aa, synthetic construct
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 48 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 49 - protein, 1018 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGEGRGSLLTCGDVEENPGPMALPVTALLLPLALLLHAARPQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGSTSGSGKPGSGEGSTKGDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 733 aa, synthetic construct
MLLLVTSLLLCELPHPAFLLIPDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKGGGGSGKPGSGEGGSQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGGGGSGKPGSDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 20 bp, synthetic construct
  source 1..20: mol_type = other DNA
gtttttgagggggaggaggg

SEQ ID NO: 52 - DNA, 20 bp, synthetic construct
  source 1..20: mol_type = other DNA
accaccggggaaattcggag

SEQ ID NO: 53 - RNA, 20 nt, synthetic construct
  source 1..20: mol_type = other RNA
ccatgggtcgggggctgctc

SEQ ID NO: 54 - RNA, 20 nt, synthetic construct
  source 1..20: mol_type = other RNA
cggggtctgccatgggtcgg

SEQ ID NO: 55 - RNA, 20 nt, synthetic construct
  source 1..20: mol_type = other RNA
ttacctgcccactgttagcc

SEQ ID NO: 56 - RNA, 20 nt, synthetic construct
  source 1..20: mol_type = other RNA
tcacccgacttctgaacgtg

SEQ ID NO: 57 - RNA, 20 nt, synthetic construct
  source 1..20: mol_type = other RNA
actcacccgacttctgaacg

SEQ ID NO: 58 - RNA, 20 nt, synthetic construct
  source 1..20: mol_type = other RNA
tgcttaccatacagccacac

SEQ ID NO: 59 - RNA, 20 nt, synthetic construct
  source 1..20: mol_type = other RNA
ctcaccttctgagaagatga

SEQ ID NO: 60 - RNA, 20 nt, synthetic construct
  source 1..20: mol_type = other RNA
ccactcacccgacttctgaa

SEQ ID NO: 61 - RNA, 20 nt, synthetic construct
  source 1..20: mol_type = other RNA
aaactcaccttctgagaaga

SEQ ID NO: 62 - RNA, 20 nt, synthetic construct
  source 1..20: mol_type = other RNA
ctcctcagttaataacgaca

SEQ ID NO: 63 - RNA, 20 nt, synthetic construct
  source 1..20: mol_type = other RNA
cactctaggagaaagaatga

SEQ ID NO: 64 - RNA, 20 nt, synthetic construct
  source 1..20: mol_type = other RNA
ctctaggagaaagaatgacg

SEQ ID NO: 65 - RNA, 20 nt, synthetic construct
  source 1..20: mol_type = other RNA
ccccatcagaatataacacc

SEQ ID NO: 66 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK